SEQUENCE LISTING (91 sequences)

SEQ ID NO: 1 - protein, 5 aa, synthetic construct
  REGION 1..5: note = Synthetic
DYYMS

SEQ ID NO: 2 - protein, 19 aa, synthetic construct
  REGION 1..19: note = Synthetic
FIKNEANGYTTESSASVKG

SEQ ID NO: 3 - protein, 14 aa, synthetic construct
  REGION 1..14: note = Synthetic
YDYYGSNYNWYFDA

SEQ ID NO: 4 - protein, 11 aa, synthetic construct
  REGION 1..11: note = Synthetic
KASQNVRTAVA

SEQ ID NO: 5 - protein, 7 aa, synthetic construct
  REGION 1..7: note = Synthetic
LASKRHT

SEQ ID NO: 6 - protein, 9 aa, synthetic construct
  REGION 1..9: note = Synthetic
LQHWIHPLT

SEQ ID NO: 7 - protein, 5 aa, synthetic construct
  REGION 1..5: note = Synthetic
AYYMH

SEQ ID NO: 8 - protein, 17 aa, synthetic construct
  REGION 1..17: note = Synthetic
RIDPEDGESKYAPKFQG

SEQ ID NO: 9 - protein, 5 aa, synthetic construct
  REGION 1..5: note = Synthetic
GSYEY

SEQ ID NO: 10 - protein, 12 aa, synthetic construct
  REGION 1..12: note = Synthetic
SASSSVSSSYLY

SEQ ID NO: 11 - protein, 7 aa, synthetic construct
  REGION 1..7: note = Synthetic
STSNLAS

SEQ ID NO: 12 - protein, 9 aa, synthetic construct
  REGION 1..9: note = Synthetic
YQWSSYPYT

SEQ ID NO: 13 - protein, 5 aa, synthetic construct
  REGION 1..5: note = Synthetic
DYYMH

SEQ ID NO: 14 - protein, 17 aa, synthetic construct
  REGION 1..17: note = Synthetic
RIDPEDGETKYAPKFQG

SEQ ID NO: 15 - protein, 4 aa, synthetic construct
  REGION 1..4: note = Synthetic
GLAY

SEQ ID NO: 16 - protein, 9 aa, synthetic construct
  REGION 1..9: note = Synthetic
HQWSSYPYT

SEQ ID NO: 17 - protein, 17 aa, synthetic construct
  REGION 1..17: note = Synthetic
RIDPEDAETKYAPKFQG

SEQ ID NO: 18 - protein, 5 aa, synthetic construct
  REGION 1..5: note = Synthetic
  VAR_SEQ 1: note = MISC_FEATURE - X is A or D
XYYMH

SEQ ID NO: 19 - protein, 17 aa, synthetic construct
  REGION 1..17: note = Synthetic
  VAR_SEQ 7: note = MISC_FEATURE - X is G or A
  VAR_SEQ 9: note = MISC_FEATURE - X is T or S
RIDPEDXEXKYAPKFQG

SEQ ID NO: 20 - skipped

SEQ ID NO: 21 - protein, 9 aa, synthetic construct
  REGION 1..9: note = Synthetic
  VAR_SEQ 1: note = note = MISC_FEATURE - X is Y or H
XQWSSYPYT

SEQ ID NO: 22 - protein, 5 aa, synthetic construct
  REGION 1..5: note = Synthetic
TYGMS

SEQ ID NO: 23 - protein, 17 aa, synthetic construct
  REGION 1..17: note = Synthetic
WINTYSGVSTCADDFKG

SEQ ID NO: 24 - protein, 14 aa, synthetic construct
  REGION 1..14: note = Synthetic
DPHSYGNSPAWFPY

SEQ ID NO: 25 - protein, 11 aa, synthetic construct
  REGION 1..11: note = Synthetic
KASQNVGISVA

SEQ ID NO: 26 - protein, 7 aa, synthetic construct
  REGION 1..7: note = Synthetic
SASNRYT

SEQ ID NO: 27 - protein, 9 aa, synthetic construct
  REGION 1..9: note = Synthetic
QQYSSYPLT

SEQ ID NO: 28 - protein, 17 aa, synthetic construct
  REGION 1..17: note = Synthetic
WINTYSGVPTYADDFQG

SEQ ID NO: 29 - protein, 14 aa, synthetic construct
  REGION 1..14: note = Synthetic
DPHYYGTSPAWFAY

SEQ ID NO: 30 - protein, 11 aa, synthetic construct
  REGION 1..11: note = Synthetic
KASQIVGIAVA

SEQ ID NO: 31 - protein, 7 aa, synthetic construct
  REGION 1..7: note = Synthetic
SASNRFT

SEQ ID NO: 32 - protein, 9 aa, synthetic construct
  REGION 1..9: note = Synthetic
QQYSTYPFT

SEQ ID NO: 33 - protein, 17 aa, synthetic construct
  REGION 1..17: note = Synthetic
WINTYSGVPTYADDFKG

SEQ ID NO: 34 - protein, 14 aa, synthetic construct
  REGION 1..14: note = Synthetic
DPHYYGSSPAWFVY

SEQ ID NO: 35 - protein, 11 aa, synthetic construct
  REGION 1..11: note = Synthetic
EASQIVGIAVA

SEQ ID NO: 36 - protein, 17 aa, synthetic construct
  REGION 1..17: note = Synthetic
YNQKEGHFPRVTTVSDL

SEQ ID NO: 37 - protein, 9 aa, synthetic construct
  REGION 1..9: note = Synthetic
QQYSAYPFT

SEQ ID NO: 38 - protein, 17 aa, synthetic construct
  REGION 1..17: note = Synthetic
  VAR_SEQ 9: note = note = MISC_FEATURE - X is S or P
  SITE 11: note = MISC_FEATURE - X is Y or C
  SITE 16: note = MISC_FEATURE - X is K or Q
WINTYSGVXTXADDFXG

SEQ ID NO: 39 - protein, 14 aa, synthetic construct
  REGION 1..14: note = Synthetic
  VAR_SEQ 4: note = MISC_FEATURE - X is Y or S
  VAR_SEQ 7: note = MISC_FEATURE - X is N or T or S
  VAR_SEQ 13: note = MISC_FEATURE - X is P or A or V
DPHXYGXSPAWFXY

SEQ ID NO: 40 - protein, 11 aa, synthetic construct
  REGION 1..11: note = Synthetic
  VAR_SEQ 1: note = note = MISC_FEATURE - X is E or K
  VAR_SEQ 5: note = MISC_FEATURE - X is N or I
  VAR_SEQ 9: note = MISC_FEATURE - X is S or A
XASQXVGIXVA

SEQ ID NO: 41 - protein, 7 aa, synthetic construct
  REGION 1..7: note = Synthetic
  VAR_SEQ 6: note = MISC_FEATURE - X is Y or F
SASNRXT

SEQ ID NO: 42 - protein, 9 aa, synthetic construct
  REGION 1..9: note = Synthetic
  VAR_SEQ 5: note = MISC_FEATURE - X is S or T or A
  VAR_SEQ 8: note = MISC_FEATURE - X is F or L
QQYSXYPXT

SEQ ID NO: 43 - protein, 5 aa, synthetic construct
  REGION 1..5: note = Synthetic
EYVLS

SEQ ID NO: 44 - protein, 17 aa, synthetic construct
  REGION 1..17: note = Synthetic
EIYPGTITTYYNEKFKG

SEQ ID NO: 45 - protein, 11 aa, synthetic construct
  REGION 1..11: note = Synthetic
FYDYDGGWFAY

SEQ ID NO: 46 - protein, 12 aa, synthetic construct
  REGION 1..12: note = Synthetic
SASSSVSSSDLH

SEQ ID NO: 47 - protein, 7 aa, synthetic construct
  REGION 1..7: note = Synthetic
GTSNLAS

SEQ ID NO: 48 - protein, 9 aa, synthetic construct
  REGION 1..9: note = Synthetic
QQWSGYPWT

SEQ ID NO: 49 - protein, 125 aa, synthetic construct
  REGION 1..125: note = Synthetic
EVKLVESGGGLVQPGGSLSLSCAASGFTFTDYYMSWVRQPPGKALEWLGFIKNEANGYTTESSASVKGRFTISRDNSQSILYLQMNALRAEDSATYYCARYDYYGSNYNWYFDAWGTGTTVTVSS

SEQ ID NO: 50 - protein, 107 aa, synthetic construct
  REGION 1..107: note = Synthetic
DIVMTQSQKFMSPSVGDRVSITCKASQNVRTAVAWYQQKPGQSPKVLIHLASKRHTGVPDRFTGSGSGTDFTLTISNVQSEDLADYFCLQHWIHPLTFGAGTKLELK

SEQ ID NO: 51 - protein, 114 aa, synthetic construct
  REGION 1..114: note = Synthetic
EVQLQQSGVEVVQPGASVKLSCTASGFNIEAYYMHWVKQRTEQGLEWIGRIDPEDGESKYAPKFQGKATMTADTSSSTAYLQLSSLTSDDTAVYYCVRGSYEYWGQGTTLTVSS

SEQ ID NO: 52 - protein, 108 aa, synthetic construct
  REGION 1..108: note = Synthetic
QIVLTQSPAIMSASPGEKVTLTCSASSSVSSSYLYWYQQKPGSSPKLWIYSTSNLASGVPPRFSGSGSGTSYSLTISSMQAEDAASYFCYQWSSYPYTFGGGTKLEIK

SEQ ID NO: 53 - protein, 113 aa, synthetic construct
  REGION 1..113: note = Synthetic
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCDRGLAYWGQGTLVTVSA

SEQ ID NO: 54 - protein, 108 aa, synthetic construct
  REGION 1..108: note = Synthetic
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPYTFGGGTKLEIK

SEQ ID NO: 55 - protein, 123 aa, synthetic construct
  REGION 1..123: note = Synthetic
QIQLVQSGPELKKPGETVKISCRASGYTFTTYGMSWVKQAPGKGLRWMGWINTYSGVSTCADDFKGRFAFSLETSATTAYLQIHNLTNEDTATYFCARDPHSYGNSPAWFPYWGQGTLVTVSA

SEQ ID NO: 56 - protein, 107 aa, synthetic construct
  REGION 1..107: note = Synthetic
DIVMTQSQKFMSTTIRDRVSITCKASQNVGISVAWYQQKSGQSPKLLIYSASNRYTGVPDRFTGSGSGTDFTLTISNMQSEDLADYFCQQYSSYPLTFGSGTKLAIK

SEQ ID NO: 57 - protein, 120 aa, synthetic construct
  REGION 1..120: note = Synthetic
QVQLQQSGPELVKPGASVKMSCKASGYTFSEYVLSWVKQRTGQGLEWIGEIYPGTITTYYNEKFKGKATLTADKSSNTAYIQLTSLTSEDSAVYFCGRFYDYDGGWFAYWGQGTLLTVSA

SEQ ID NO: 58 - protein, 108 aa, synthetic construct
  REGION 1..108: note = Synthetic
ENVLTQSPEKMAVSLGQKVTMTCSASSSVSSSDLHWYQQKSGASPKPLIHGTSNLASGVPARFSGSGSGTSYSLTISSVEAEDAATYYCQQWSGYPWTFGGGTNLEIK

SEQ ID NO: 59 - protein, 123 aa, synthetic construct
  REGION 1..123: note = Synthetic
QIQLVQSGPELKKPGETVKISCKASGYTFTTYGMSWVKQAPGKGLKWMVWINTYSGVPTYADDFQGRFAFSLETSASTSYLQINNLRNEDTATYFCARDPHYYGTSPAWFAYWGQGTLVTVSA

SEQ ID NO: 60 - protein, 107 aa, synthetic construct
  REGION 1..107: note = Synthetic
DIVMTQSQKFMSTTIGDRVIITCKASQIVGIAVAWYQQKPGQSPKLLIYSASNRFTGVPDRFTGSGSGTDFTLTISNMQSEDLADYFCQQYSTYPFTFGSGTKLEIK

SEQ ID NO: 61 - protein, 123 aa, synthetic construct
  REGION 1..123: note = Synthetic
QIQLVQSGPELKKPGETVKISCKASGYTFTTYGMSWVKQAPGKGLKWMVWINTYSGVPTYADDFKGRFAFSLETSASTSYLQINNLKNEDTATYFCARDPHYYGSSPAWFVYWGQGTLVTVSA

SEQ ID NO: 62 - protein, 107 aa, synthetic construct
  REGION 1..107: note = Synthetic
DIVMTQSQKFMSTTIGDRVSITCEASQIVGIAVAWYQQKPGQSPKLLIYSASNRYTGVPDRFTGSGSGTDFTLTISNMQSEDLANYFCQQYSAYPFTFGSGTKLEVK

SEQ ID NO: 63 - protein, 113 aa, synthetic construct
  REGION 1..113: note = Synthetic
EVQLVQSGAEVKKPGATVKISCKVSGFNIKDYYMHWVQQAPGKGLEWIGRIDPEDAETKYAPKFQGRVTITADTSTNTAYMELSSLRSEDTAVYYCDRGLAYWGQGTLVTVSS

SEQ ID NO: 64 - protein, 108 aa, synthetic construct
  REGION 1..108: note = Synthetic
EIVLTQSPATLSLSPGERATLSCSASSSVSSSYLYWYQQKPGQAPKLWIYSTSNLASGIPARFSGSGSGTDYTLTISSLEPEDFAVYYCHQWSSYPYTFGQGTKLEIK

SEQ ID NO: 65 - protein, 113 aa, synthetic construct
  REGION 1..113: note = Synthetic
EVQLVQSGAEVKKPGATVKISCKVSGFNIKDYYMHWVQQAPGKGLEWIGRIDPEDAETKYAPKFQGRVTITADTSTDTAYMELSSLRSEDTAVYYCDRGLAYWGQGTLVTVSS

SEQ ID NO: 66 - protein, 108 aa, synthetic construct
  REGION 1..108: note = Synthetic
EIVLTQSPATLSLSPGERATLSCSASSSVSSSYLYWYQQKPGQAPKLWIYSTSNLASGIPARFSGSGSGTDFTLTISSLEPEDFAVYYCHQWSSYPYTFGQGTKLEIK

SEQ ID NO: 67 - protein, 113 aa, synthetic construct
  REGION 1..113: note = Synthetic
EVQLVQSGAEVKKPGATVKISCKASGFNIKDYYMHWVQQAPGKGLEWIGRIDPEDAETKYAPKFQGRVTITADTSTNTAYMELSSLRSEDTAVYYCDRGLAYWGQGTLVTVSS

SEQ ID NO: 68 - protein, 120 aa, synthetic construct
  REGION 1..120: note = Synthetic
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 69 - protein, 112 aa, synthetic construct
  REGION 1..112: note = Synthetic
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 70 - protein, 120 aa, synthetic construct
  REGION 1..120: note = Synthetic
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 71 - protein, 107 aa, synthetic construct
  REGION 1..107: note = Synthetic
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLELK

SEQ ID NO: 72 - protein, 7 aa, synthetic construct
  REGION 1..7: note = Synthetic
SGAGTEL

SEQ ID NO: 73 - protein, 30 aa, synthetic construct
  REGION 1..30: note = Synthetic
EVQLVQSGAEVKKPGATVKISCKVSGFNIK

SEQ ID NO: 74 - protein, 14 aa, synthetic construct
  REGION 1..14: note = Synthetic
WVQQAPGKGLEWIG

SEQ ID NO: 75 - protein, 32 aa, synthetic construct
  REGION 1..32: note = Synthetic
RVTITADTSTNTAYMELSSLRSEDTAVYYCDR

SEQ ID NO: 76 - protein, 11 aa, synthetic construct
  REGION 1..11: note = Synthetic
WGQGTLVTVSS

SEQ ID NO: 77 - protein, 23 aa, synthetic construct
  REGION 1..23: note = Synthetic
EIVLTQSPATLSLSPGERATLSC

SEQ ID NO: 78 - protein, 15 aa, synthetic construct
  REGION 1..15: note = Synthetic
WYQQKPGQAPKLWIY

SEQ ID NO: 79 - protein, 32 aa, synthetic construct
  REGION 1..32: note = Synthetic
GIPARFSGSGSGTDYTLTISSLEPEDFAVYYC

SEQ ID NO: 80 - protein, 10 aa, synthetic construct
  REGION 1..10: note = Synthetic
FGQGTKLEIK

SEQ ID NO: 81 - protein, 32 aa, synthetic construct
  REGION 1..32: note = Synthetic
GIPARFSGSGSGTDFTLTISSLEPEDFAVYYC

SEQ ID NO: 82 - protein, 32 aa, synthetic construct
  REGION 1..32: note = Synthetic
RVTITADTSTDTAYMELSSLRSEDTAVYYCDR

SEQ ID NO: 83 - protein, 30 aa, synthetic construct
  REGION 1..30: note = Synthetic
EVQLVQSGAEVKKPGATVKISCKASGFNIK

SEQ ID NO: 84 - protein, 30 aa, synthetic construct
  REGION 1..30: note = Synthetic
  SITE 24: note = MISC_FEATURE - X is A or V
EVQLVQSGAEVKKPGATVKISCKXSGFNIK

SEQ ID NO: 85 - protein, 32 aa, synthetic construct
  REGION 1..32: note = Synthetic
  VAR_SEQ 11: note = MISC_FEATURE - X is N or D
RVTITADTSTXTAYMELSSLRSEDTAVYYCDR

SEQ ID NO: 86 - protein, 32 aa, synthetic construct
  REGION 1..32: note = Synthetic
  VAR_SEQ 15: note = MISC_FEATURE - X is Y or F
GIPARFSGSGSGTDXTLTISSLEPEDFAVYYC

SEQ ID NO: 87 - protein, 11 aa, synthetic construct
  REGION 1..11: note = Synthetic
TNVDPVGESVS

SEQ ID NO: 88 - protein, 117 aa, synthetic construct
  REGION 1..117: note = Synthetic
QVQLVQSGAEVKKPGASVKVSCKASGYTFTNWVHWVRQAPGQGLEWMGEINPTNARSNYNEKFKKRVTMTRDTSTSTVYMELSSLRSEDTAVYYCARIYYGNSFAHWGQGTLVTVSS

SEQ ID NO: 89 - protein, 113 aa, synthetic construct
  REGION 1..113: note = Synthetic
DIVMTQSPDSLAVSLGERATINCKSSQSLLNAGNQKNYLTWYQQKPGQPPKLLIYWSSTRESGVPDRFSGSGSGTDFTLTISSLQAEDVAVYYCQNNYYYPLTFGGGTKLEIK

SEQ ID NO: 90 - protein, 12 aa, synthetic construct
  REGION 1..12: note = Synthetic
TNVDPVGESVSY

SEQ ID NO: 91 - protein, 112 aa, synthetic construct
  REGION 1..112: note = Synthetic
EVQVVESGGGLVQSGGSLKLSCAGSGFTESAGFMVWHRQVPGKERELVALIATPSGSTNYADSVKGRFTISRDNGKNTVYLQMNSLKPEDTAVYYCNIRGYWGQGTLVTVSS

(19) United States
(12) Patent Application Publication
(10) Pub. No.: US 2025/0270315 A1
(43) Pub. Date: Aug. 28, 2025

(54) NOVEL ANTI-SIRPA ANTIBODIES

(71) Applicants: ELPISCIENCE (SUZHOU) BIOPHARMA, LTD., Suzhou, Jiangsu (CN); ELPISCIENCE BIOPHARMA, LTD., Shanghai (CN)

(72) Inventors: Hongtao LU, Shanghai (CN); Xiaofeng NIU, Shanghai (CN); Fengli WANG, Shanghai (CN); Chunnian WANG, Shanghai (CN); Jinfeng ZHAO, Shanghai (CN); Roumei XING, Shanghai (CN); Haiying WANG, Shanghai (CN); Jingfeng YU, Shanghai (CN); Lei LI, Shanghai (CN); Zhihao WU, Shanghai (CN); Rui GAO, Shanghai (CN); Yangsheng QIU, Shanghai (CN)

(21) Appl. No.: 18/292,922

(22) PCT Filed: Jul. 28, 2022
(86) PCT No.: PCT/US2022/074286

§ 371 Date: Jan. 28, 2024

(30) Foreign Application Priority Data
Jul. 28, 2021 (WO) PCT/CN2021/109041
Jul. 8, 2022 (WO) PCT/CN2022/104562

(51) Int. Cl. C07K 16/28; A61K 47/68; A61P 35/00; A61P 37/04

(52) U.S. Cl. CPC C07K 16/2803; A61K 47/6849; A61P 35/00; A61P 37/04; C07K 2317/24; C07K 2317/31; C07K 2317/52; C07K 2317/565; C07K 2317/567; C07K 2317/732; C07K 2317/76; C07K 2317/92

(57) ABSTRACT

The present disclosure provides anti-SIRPα antibodies or antigen-binding fragments thereof, isolated polynucleotides encoding the same, pharmaceutical compositions comprising the same and the uses thereof.

### FIELD OF THE INVENTION

The present disclosure generally relates to novel anti-SIRPα antibodies. The instant application contains a sequence listing which has been submitted electronically in xml format and is herein incorporated by reference in its entirety. Said xml copy, created on Sep. 2, 2024 is named 075431-8009US01 2024-09-02_sequence_listing.xml and is 119 KB in size.

### BACKGROUND

Signal-regulatory protein alpha (SIRPα), is an inhibitory receptor expressed primarily on myeloid cells and dendritic cells. In addition to SIRPα, the SIRPs family also includes several other transmembrane glycoproteins, including, SIRPβ and SIRPγ. Each member of the SIRPs family contains 3 similar extracellular Ig-like domains with distinct transmembrane and cytoplasmic domains. CD47 is a broadly expressed transmembrane glycoprotein with an extracellular N-terminal IgV domain, five transmembrane domains, and a short C-terminal intracellular tail. CD47 functions as a cellular ligand for SIRPα. Binding of CD47 to SIRPα delivers a “don't eat me” signal to suppress phagocytosis, and blocking the CD47 mediated engagement of SIRPα on a phagocyte can cause removal of live cells bearing “eat me” signals. Tumor cells frequently overexpress CD47 to evade macrophage-mediated destruction. The interaction of CD47 and SIRPα has been shown to be involved in the regulation of macrophage-mediated phagocytosis (Takenaka et al., Nature Immunol., 8 (12): 1313-1323, 2007). In a diverse range of preclinical models, therapies that block the interaction of CD47 and SIRPα stimulate phagocytosis of cancer cells in vitro and anti-tumor immune responses in vivo. Currently, multiple agents targeting CD47 (anti-CD47 antibodies and SIRPα fusion proteins) have proceeded to clinical trials. However, these agents have been associated with hemolytic anemia and thrombocytopenia. In addition to safety issues, universal expression of CD47 may also cause antigen sink, which leads to reduced efficacy.

Needs remain for novel anti-SIRPα antibodies.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one aspect, the present disclosure provides an antibody or an antigen-binding fragment thereof capable of specifically binding to human SIRPα, comprising a heavy chain variable region comprising HCDR1, HCDR2 and HCDR3, and/or a light chain variable region comprising LCDR1, LCDR2 and LCDR3, wherein
a) the HCDR1 comprises an amino acid sequence of DYYMS (SEQ ID NO: 1), and/or
   the HCDR2 comprises an amino acid sequence of FIKNEANGYTTESSASVKG (SEQ ID NO: 2), and/or
   the HCDR3 comprises an amino acid sequence of YDYYGSNYNWYFDA (SEQ ID NO: 3), and/or
   the LCDR1 comprises an amino acid sequence of KASQNVRTAVA (SEQ ID NO: 4), and/or
   the LCDR2 comprises an amino acid sequence of LASKRHT (SEQ ID NO: 5), and/or
   the LCDR3 comprises an amino acid sequence of LQHWIHPLT (SEQ ID NO: 6),
b) the HCDR1 comprises an amino acid sequence of X₁YYMH (SEQ ID NO: 18), and/or
   the HCDR2 comprises an amino acid sequence of RIDPEDX₂EX₃KYAPKFQG (SEQ ID NO: 19), and/or
   the HCDR3 comprises an amino acid sequence of GX₁₈X₄X₅Y (SEQ ID NO: 20), and/or
   the LCDR1 comprises an amino acid sequence of SASSSVSSSYLY (SEQ ID NO: 10), and/or
   the LCDR2 comprises an amino acid sequence of STSNLAS (SEQ ID NO: 11), and/or
   the LCDR3 comprises an amino acid sequence of X₆QWSSYPYT (SEQ ID NO: 21),
c) the HCDR1 comprises an amino acid sequence of TYGMS (SEQ ID NO: 22), and/or
   the HCDR2 comprises an amino acid sequence of WINTYSGVX₁₉TX₇ADDFX&G (SEQ ID NO: 38), and/or
   the HCDR3 comprises an amino acid sequence of DPHX₉YGX₁₀SPAWFX₁₁Y (SEQ ID NO: 39), and/or
   the LCDR1 comprises an amino acid sequence of X₁₂ASQX₁₃ VGIX₁₄VA (SEQ ID NO: 40), and/or
   the LCDR2 comprises an amino acid sequence of SASNRX₁ST (SEQ ID NO: 41), and/or
   the LCDR3 comprises an amino acid sequence of QQYSX₁₆YPX₁₇T (SEQ ID NO: 42),
d) the HCDR1 comprises an amino acid sequence of EYVLS (SEQ ID NO: 43), and/or
   the HCDR2 comprises an amino acid sequence of EIYPGTITTYYNEKFKG (SEQ ID NO: 44), and/or
   the HCDR3 comprises an amino acid sequence of FYDYDGGWFAY (SEQ ID NO: 45), and/or
   the LCDR1 comprises an amino acid sequence of SASSSVSSSDLH (SEQ ID NO: 46), and/or
   the LCDR2 comprises an amino acid sequence of GTSNLAS (SEQ ID NO: 47), and/or
   the LCDR3 comprises an amino acid sequence of QQWSGYPWT (SEQ ID NO: 48),
      wherein X₁ is A or D; X₂ is G or A; X₃ is T or S; X₄ is L or Y; X₅ is E or A; X₆ is Y or H; X₇ is Y or C; X₈ is K or Q; X₉ is Y or S; X₁₀ is N or T or S; X₁₁ is P or A or V; X₁₂ is E or K; X₁₃ is N or I; X₁₄ is S or A; X₁₅ is Y or F; X₁₆ is S or T or A; X₁₇ is F or L; X₁₈ is S or absent; X₁₉ is S or P.

In certain embodiments, the antibody or an antigen-binding fragment thereof provided herein comprises:
a) the HCDR1 comprises an amino acid sequence of X₁YYMH (SEQ ID NO: 18), and/or
b) the HCDR2 comprises an amino acid sequence of RIDPEDX₂EX₃KYAPKFQG (SEQ ID NO: 19), and/or
c) the HCDR3 comprises an amino acid sequence of GX₁₈X₄X₅Y (SEQ ID NO: 20), and/or
d) the LCDR1 comprises an amino acid sequence of SASSSVSSSYLY (SEQ ID NO: 10), and/or
e) the LCDR2 comprises an amino acid sequence of STSNLAS (SEQ ID NO: 11), and/or
f) the LCDR3 comprises an amino acid sequence of X₆QWSSYPYT (SEQ ID NO: 21),
   wherein X₁ is A or D; X₂ is G or A; X₃ is T or S; X₄ is L or Y; X₅ is E or A; X₆ is Y or H; and X₁₈ is S or absent.

In certain embodiments, the antibody or an antigen-binding fragment thereof provided herein comprises:
a) the HCDR1 comprises an amino acid sequence of AYYMH (SEQ ID NO: 7) or DYYMH (SEQ ID NO: 13), and/or
b) the HCDR2 comprises an amino acid sequence selected from the group consisting of RIDPEDGESKYAPKFQG (SEQ ID NO: 8), RIDPEDGETKYAPKFQG (SEQ ID NO: 14) and RIDPEDAETKYAPKFQG (SEQ ID NO: 17), and/or
c) the HCDR3 comprises an amino acid sequence of GSYEY (SEQ ID NO: 9) or GLAY (SEQ ID NO: 15), and/or
d) the LCDR1 comprises an amino acid sequence of SASSSVSSSYLY (SEQ ID NO: 10), and/or
e) the LCDR2 comprises an amino acid sequence of STSNLAS (SEQ ID NO: 11), and/or
f) the LCDR3 comprises an amino acid sequence of YQWSSYPYT (SEQ ID NO: 12) or HQWSSYPYT (SEQ ID NO: 16).

In certain embodiments, the antibody or an antigen-binding fragment thereof provided herein comprises:
a) the HCDR1 comprises an amino acid sequence of TYGMS (SEQ ID NO: 22), and/or
b) the HCDR2 comprises an amino acid sequence of WINTYSGVX₁₉TX₇ADDFX&G (SEQ ID NO: 38), and/or
c) the HCDR3 comprises an amino acid sequence of DPHX, YGX₁₀SPAWFX₁₁Y (SEQ ID NO: 39), and/or
d) the LCDR1 comprises an amino acid sequence of X₁₂ASQX₁₃ VGIX₁₄VA (SEQ ID NO: 40), and/or
e) the LCDR2 comprises an amino acid sequence of SASNRX₁ST (SEQ ID NO: 41), and/or
f) the LCDR3 comprises an amino acid sequence of QQYSX 16 YPX₁₇T (SEQ ID NO: 42),
wherein X₇ is Y or C; X₈ is K or Q; X₉ is Y or S; X₁₀ is N or T or S; X₁₁ is P or A or V; X₁₂ is E or K; X₁₃ is N or I; X₁₄ is S or A; X₁₅ is Y or F; X₁₆ is S or Tor A; X₁₇ is F or L; and X₁₉ is S or P.

In certain embodiments, the antibody or an antigen-binding fragment thereof provided herein comprises:
a) the HCDR1 comprises an amino acid sequence of TYGMS (SEQ ID NO: 22), and/or
b) the HCDR2 comprises an amino acid sequence selected from the group consisting of WINTYSGVSTCADDFKG (SEQ ID NO: 23), WINTYSGVPTYADDFQG (SEQ ID NO: 28) and WINTYSGVPTYADDFKG (SEQ ID NO: 33), and/or
c) the HCDR3 comprises an amino acid sequence selected from the group consisting of DPHSYGNSPAWFPY (SEQ ID NO: 24), DPHYYGTSPAWFAY (SEQ ID NO: 29) and DPHYYGSSPAWFVY (SEQ ID NO: 34), and/or
d) the LCDR1 comprises an amino acid sequence selected from the group consisting of KASQNVGISVA (SEQ ID NO: 25), KASQIVGIAVA (SEQ ID NO: 30) and EASQIVGIAVA (SEQ ID NO: 35), and/or
e) the LCDR2 comprises an amino acid sequence selected from the group consisting of SASNRYT (SEQ ID NO: 26) and SASNRFT (SEQ ID NO: 31), and/or
f) the LCDR3 comprises an amino acid sequence selected from the group consisting of QQYSSYPLT (SEQ ID NO: 27), QQYSTYPFT (SEQ ID NO: 32) and QQYSAYPFT (SEQ ID NO: 37).

In certain embodiments, the heavy chain variable region comprises:
a) a HCDR1 comprising the sequence of SEQ ID NO: 1, a HCDR2 comprising the sequence of SEQ ID NO: 2, and a HCDR3 comprising the sequence of SEQ ID NO: 3; or
b) a HCDR1 comprising the sequence of SEQ ID NO: 7, a HCDR2 comprising the sequence of SEQ ID NO: 8, and a HCDR3 comprising the sequence of SEQ ID NO: 9; or
c) a HCDR1 comprising the sequence of SEQ ID NO: 13, a HCDR2 comprising the sequence of SEQ ID NO: 14 or SEQ ID NO: 17, and a HCDR3 comprising the sequence of SEQ ID NO: 15; or
d) a HCDR1 comprising the sequence of SEQ ID NO: 22, a HCDR2 comprising the sequence of SEQ ID NO: 23, and a HCDR3 comprising the sequence of SEQ ID NO: 24; or
e) a HCDR1 comprising the sequence of SEQ ID NO: 22, a HCDR2 comprising the sequence of SEQ ID NO: 28, and a HCDR3 comprising the sequence of SEQ ID NO: 29; or
f) a HCDR1 comprising the sequence of SEQ ID NO: 22, a HCDR2 comprising the sequence of SEQ ID NO: 33, and a HCDR3 comprising the sequence of SEQ ID NO: 34; or
g) a HCDR1 comprising the sequence of SEQ ID NO: 43, a HCDR2 comprising the sequence of SEQ ID NO: 44, and a HCDR3 comprising the sequence of SEQ ID NO: 45.

In certain embodiments, the light chain variable region comprises:
a) a LCDR1 comprising the sequence of SEQ ID NO: 4, a LCDR2 comprising the sequence of SEQ ID NO: 5, and a LCDR3 comprising the sequence of SEQ ID NO: 6; or
b) a LCDR1 comprising the sequence of SEQ ID NO: 10, a LCDR2 comprising the sequence of SEQ ID NO: 11, and a LCDR3 comprising the sequence of SEQ ID NO: 12; or
c) a LCDR1 comprising the sequence of SEQ ID NO: 10, a LCDR2 comprising the sequence of SEQ ID NO: 11, and a LCDR3 comprising the sequence of SEQ ID NO: 16; or
d) a LCDR1 comprising the sequence of SEQ ID NO: 25, a LCDR2 comprising the sequence of SEQ ID NO: 26, and a LCDR3 comprising the sequence of SEQ ID NO: 27; or
e) a LCDR1 comprising the sequence of SEQ ID NO: 30, a LCDR2 comprising the sequence of SEQ ID NO: 31, and a LCDR3 comprising the sequence of SEQ ID NO: 32; or
f) a LCDR1 comprising the sequence of SEQ ID NO: 35, a LCDR2 comprising the sequence of SEQ ID NO: 26, and a LCDR3 comprising the sequence of SEQ ID NO: 37; or
g) a LCDR1 comprising the sequence of SEQ ID NO: 46, a LCDR2 comprising the sequence of SEQ ID NO: 47, and a LCDR3 comprising the sequence of SEQ ID NO: 48.

In certain embodiments, the antibody or an antigen-binding fragment thereof provided herein comprises:
a) a HCDR1 comprising the sequence of SEQ ID NO: 1, a HCDR2 comprising the sequence of SEQ ID NO: 2, and a HCDR3 comprising the sequence of SEQ ID NO: 3, a LCDR1 comprising the sequence of SEQ ID NO: 4, a LCDR2 comprising the sequence of SEQ ID NO: 5, and a LCDR3 comprising the sequence of SEQ ID NO: 6; or
b) a HCDR1 comprising the sequence of SEQ ID NO: 7, a HCDR2 comprising the sequence of SEQ ID NO: 8, and a HCDR3 comprising the sequence of SEQ ID NO: 9, a LCDR1 comprising the sequence of SEQ ID NO: 10, a LCDR2 comprising the sequence of SEQ ID NO: 11, and a LCDR3 comprising the sequence of SEQ ID NO: 12; or
c) a HCDR1 comprising the sequence of SEQ ID NO: 13, a HCDR2 comprising the sequence of SEQ ID NO: 14 or SEQ ID NO: 17, and a HCDR3 comprising the sequence of SEQ ID NO: 15, a LCDR1 comprising the sequence of SEQ ID NO: 10, a LCDR2 comprising the sequence of SEQ ID NO: 11, and a LCDR3 comprising the sequence of SEQ ID NO: 16; or
d) a HCDR1 comprising the sequence of SEQ ID NO: 22, a HCDR2 comprising the sequence of SEQ ID NO: 23, and a HCDR3 comprising the sequence of SEQ ID NO: 24, a LCDR1 comprising the sequence of SEQ ID NO: 25, a LCDR2 comprising the sequence of SEQ ID NO: 26, and a LCDR3 comprising the sequence of SEQ ID NO: 27; or
e) a HCDR1 comprising the sequence of SEQ ID NO: 22, a HCDR2 comprising the sequence of SEQ ID NO: 28, and a HCDR3 comprising the sequence of SEQ ID NO: 29, a LCDR1 comprising the sequence of SEQ ID NO: 30, a LCDR2 comprising the sequence of SEQ ID NO: 31, and a LCDR3 comprising the sequence of SEQ ID NO: 32; or
f) a HCDR1 comprising the sequence of SEQ ID NO: 22, a HCDR2 comprising the sequence of SEQ ID NO: 33, and a HCDR3 comprising the sequence of SEQ ID NO: 34, a LCDR1 comprising the sequence of SEQ ID NO: 35, a LCDR2 comprising the sequence of SEQ ID NO: 26, and a LCDR3 comprising the sequence of SEQ ID NO: 37; or
g) a HCDR1 comprising the sequence of SEQ ID NO: 43, a HCDR2 comprising the sequence of SEQ ID NO: 44, and a HCDR3 comprising the sequence of SEQ ID NO: 45, a LCDR1 comprising the sequence of SEQ ID NO: 46, a LCDR2 comprising the sequence of SEQ ID NO: 47, and a LCDR3 comprising the sequence of SEQ ID NO: 48.

In certain embodiments, the antibody or an antigen-binding fragment thereof provided herein further comprises one or more of heavy chain HFR1, HFR2, HFR3 and HFR4, and/or one or more of light chain LFR1, LFR2, LFR3 and LFR4, wherein:
a) the HFR1 comprises EVQLVQSGAEVKKPGATVKISCKX₂₀SGFNIK (SEQ ID NO: 84) or a homologous sequence of at least 80% sequence identity thereof, and/or
b) the HFR2 comprises WVQQAPGKGLEWIG (SEQ ID NO: 74) or a homologous sequence of at least 80% sequence identity thereof, and/or
c) the HFR3 sequence comprises RVTITADTSTX₂₁ TAYMELSSLRSEDTAVYYCDR (SEQ ID NO: 85) or a homologous sequence of at least 80% sequence identity thereof, and/or
d) the HFR4 comprises WGQGTLVTVSS (SEQ ID NO: 76) or a homologous sequence of at least 80% sequence identity thereof, and/or
e) the LFR1 comprises EIVLTQSPATLSLSPGERATLSC (SEQ ID NO: 77) or a homologous sequence of at least 80% sequence identity thereof, and/or
f) the LFR2 comprises WYQQKPGQAPKLWIY (SEQ ID NO: 78) or a homologous sequence of at least 80% sequence identity thereof, and/or
g) the LFR3 comprises GIPARFSGSGSGTDX₂₂TLTISSLEPEDFAVYYC (SEQ ID NO: 86) or a homologous sequence of at least 80% sequence identity thereof, and/or
h) the LFR4 comprises FGQGTKLEIK (SEQ ID NO: 80) or a homologous sequence of at least 80% sequence identity thereof,
   wherein X₂₀ is A or V; X₂₁ is N or D; X₂₂ is Y or F.

In certain embodiments,
a) the HFR1 comprises EVQLVQSGAEVKKPGATVKISCKASGFNIK (SEQ ID NO: 83) or EVQLVQSGAEVKKPGATVKISCKVSGFNIK (SEQ ID NO: 73), or a homologous sequence of at least 80% sequence identity thereof, and/or
b) the HFR2 comprises WVQQAPGKGLEWIG (SEQ ID NO: 74) or a homologous sequence of at least 80% sequence identity thereof, and/or
c) the HFR3 sequence comprises RVTITADTSTNTAYMELSSLRSEDTAVYYCDR (SEQ ID NO: 75) or RVTITADTSTDTAYMELSSLRSEDTAVYYCDR (SEQ ID NO: 82) or a homologous sequence of at least 80% sequence identity thereof, and/or
d) the HFR4 comprises WGQGTLVTVSS (SEQ ID NO: 76) or a homologous sequence of at least 80% sequence identity thereof, and/or
e) the LFR1 comprises EIVLTQSPATLSLSPGERATLSC (SEQ ID NO: 77) or a homologous sequence of at least 80% sequence identity thereof, and/or
f) the LFR2 comprises WYQQKPGQAPKLWIY (SEQ ID NO: 78) or a homologous sequence of at least 80% sequence identity thereof, and/or
g) the LFR3 comprises GIPARFSGSGSGTDYTLTISSLEPEDFAVYYC (SEQ ID NO: 79) or GIPARFSGSGSGTDFTLTISSLEPEDFAVYYC (SEQ ID NO: 81) or a homologous sequence of at least 80% sequence identity thereof, and/or
h) the LFR4 comprises FGQGTKLEIK (SEQ ID NO: 80) or a homologous sequence of at least 80% sequence identity thereof.

In certain embodiments, the heavy chain variable region comprises the sequence selected from the group consisting of SEQ ID NO: 63, SEQ ID NO: 65 and SEQ ID NO: 67, and a homologous sequence thereof having at least 80% sequence identity yet retaining specific binding affinity to human SIRPα.

In certain embodiments, the light chain variable region comprises the sequence selected from the group consisting of SEQ ID NO: 64 and SEQ ID NO: 66, and a homologous sequence thereof having at least 80% sequence identity yet retaining specific binding affinity to human SIRPα.

In certain embodiments,
a) the heavy chain variable region comprises the sequence of SEQ ID NO: 49 and the light chain variable region comprises the sequence of SEQ ID NO: 50; or
b) the heavy chain variable region comprises the sequence of SEQ ID NO: 51 and the light chain variable region comprises the sequence of SEQ ID NO: 52; or
c) the heavy chain variable region comprises the sequence of SEQ ID NO: 53 and the light chain variable region comprises the sequence of SEQ ID NO: 54; or
d) the heavy chain variable region comprises the sequence of SEQ ID NO: 55 and the light chain variable region comprises the sequence of SEQ ID NO: 56; or
e) the heavy chain variable region comprises the sequence of SEQ ID NO: 57 and the light chain variable region comprises the sequence of SEQ ID NO: 58; or
f) the heavy chain variable region comprises the sequence of SEQ ID NO: 59 and the light chain variable region comprises the sequence of SEQ ID NO: 60; or
g) the heavy chain variable region comprises the sequence of SEQ ID NO: 61 and the light chain variable region comprises the sequence of SEQ ID NO: 62; or
h) the heavy chain variable region comprises the sequence of SEQ ID NO: 63 and the light chain variable region comprises the sequence of SEQ ID NO: 64; or
i) the heavy chain variable region comprises the sequence of SEQ ID NO: 63 and the light chain variable region comprises the sequence of SEQ ID NO: 66; or
j) the heavy chain variable region comprises the sequence of SEQ ID NO: 65 and the light chain variable region comprises the sequence of SEQ ID NO: 64; or
k) the heavy chain variable region comprises the sequence of SEQ ID NO: 67 and the light chain variable region comprises the sequence of SEQ ID NO: 64; or
l) the heavy chain variable region comprises the sequence of SEQ ID NO: 67 and the light chain variable region comprises the sequence of SEQ ID NO: 66.

In certain embodiments, the antibody or an antigen-binding fragment thereof provided herein further comprises one or more amino acid residue substitutions or modifications yet retains specific binding affinity to human SIRPα.

In certain embodiments, at least one of the substitutions or modifications is in one or more of the CDR sequences, and/or in one or more of the non-CDR sequences of the heavy chain variable region or light chain variable region.

In certain embodiments, the antibody or an antigen-binding fragment thereof provided herein further comprises an Fc region, optionally an Fc region of human immunoglobulin (Ig), or optionally an Fc region of human IgG.

In certain embodiments, the Fc region is derived from human IgG4.

In certain embodiments, the Fc region derived from human IgG4 comprises a S228P mutation and/or a L235E mutation.

In certain embodiments, the antibody or an antigen-binding fragment thereof provided herein is humanized.

In certain embodiments, the antibody or an antigen-binding fragment thereof provided herein is a monoclonal antibody, a bispecific antibody, a multi-specific antibody, a recombinant antibody, a chimeric antibody, a labeled antibody, a bivalent antibody, an anti-idiotypic antibody or a fusion protein.

In certain embodiments, the antibody or an antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In certain embodiments, the antibody or an antigen-binding fragment thereof provided herein have one or more properties selected from the group consisting of:
a) capable of completely blocking interaction between SIRP-alpha v1 and CD47,
b) capable of blocking interaction between SIRP-alpha v1 and CD47 at an IC₅₀ of no more than 10 nM (or no more than 5 nM), as measured by competitive ELISA or at an IC₅₀ of no more than 0.6 nM (or no more than 0.5 nM), as measured by competitive FACS;
c) capable of completely blocking interaction between SIRP-alpha v2 and CD47,
d) capable of blocking interaction between SIRP-alpha v2 and CD47 at an IC₅₀ of no more than 10 nM (or no more than 5 nM), as measured by competitive ELISA or at an IC₅₀ of no more than 0.8 nM (or no more than 0.7 nM), as measured by competitive FACS;
e) having no significant inhibition on IFNγ secretion by T cells, CD4⁺ T cell proliferation or CD8⁺ T cell proliferation;
f) capable of blocking CD47 mediated SHP1 recruitment to SIRP alpha;
g) capable of increasing antibody-dependent cellular phagocytosis (ADCP) effect of a target antibody;
h) capable of binding to an epitope comprising an amino acid sequence selected from the group consisting of YNQKEGHFPRVTTVSDL (SEQ ID NO: 36), SGAGTEL (SEQ ID NO: 72), TNVDPVGESVS (SEQ ID NO: 87) and TNVDPVGESVSY (SEQ ID NO: 90).

In another aspect, the present disclosure also provides an antibody or an antigen-binding fragment thereof which competes for binding to human SIRPα with an antibody comprising a heavy chain variable region comprising the sequence of SEQ ID NO: 53, and a light chain variable region comprising the sequence of SEQ ID NO: 54.

In another aspect, the present disclosure also provides an antibody or an antigen-binding fragment thereof which competes for binding to human SIRPα with an antibody comprising a heavy chain variable region comprising the sequence of SEQ ID NO: 55, and a light chain variable region comprising the sequence of SEQ ID NO: 56.

In another aspect, the present disclosure also provides an antibody or an antigen-binding fragment thereof which competes for binding to human SIRPα with an antibody comprising a heavy chain variable region comprising the sequence of SEQ ID NO: 61, and a light chain variable region comprising the sequence of SEQ ID NO: 62.

In certain embodiments, the antibody or an antigen-binding fragment thereof provided herein is bispecific.

In certain embodiments, the antibody or an antigen-binding fragment thereof provided herein is capable of specifically binding to a second antigen other than SIRPα.

In certain embodiments, the second antigen is a tumor antigen, tumor surface antigen, an inflammatory antigen, an antigen of an infectious microorganism.

In certain embodiments, the antibody or an antigen-binding fragment thereof provided herein is capable of specifically binding to a second epitope on SIRPα.

In certain embodiments, the antibody or an antigen-binding fragment thereof provided herein is linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety, or other anticancer drugs.

In another aspect, the present disclosure also provides a pharmaceutical composition comprising the antibody or an antigen-binding fragment thereof provided herein, and one or more pharmaceutically acceptable carriers.

In another aspect, the present disclosure also provides an isolated polynucleotide encoding the antibody or an antigen-binding fragment thereof provided herein.

In another aspect, the present disclosure also provides a vector comprising the isolated polynucleotide provided herein.

In another aspect, the present disclosure also provides a host cell comprising the vector provided herein.

In another aspect, the present disclosure also provides a method of expressing the antibody or an antigen-binding fragment thereof provided herein, comprising culturing the host cell provided herein under the condition at which the vector provided herein is expressed.

In another aspect, the present disclosure also provides a method of inducing phagocytosis in vitro, comprising contacting a target cell with a SIRPα positive phagocytic cell sample in the presence of the antibody or an antigen-binding fragment thereof provided herein or the pharmaceutical composition provided herein, optionally in combination with a target antibody that specifically binds to a target antigen on the target cell, thereby inducing phagocytosis of the target cell by the SIRPα positive phagocytic cell.

In another aspect, the present disclosure also provides a method of inducing phagocytosis of a target cell in a subject, comprising administering to the subject the antibody or an antigen-binding fragment thereof provided herein or the pharmaceutical composition provided herein, optionally in combination with a target antibody that specifically binds to a target antigen on the target cell, in a dose effective to induce phagocytosis of the target cell.

In another aspect, the present disclosure also provides a method of increasing antibody-dependent cellular phagocytosis (ADCP) effect of a target antibody on a target cell in a subject, comprising:
administering to the subject a therapeutically effective amount of the antibody or an antigen-binding fragment thereof provided herein or the pharmaceutical composition provided herein, in combination with the target antibody, thereby increasing ADCP of the target antibody on the target cell,
wherein the target antibody binds to a target antigen expressed on the target cell.

In another aspect, the present disclosure also provides a method of treating, preventing or alleviating a disease disorder or condition that can be benefited from induced phagocytosis of a target cell in a subject, comprising administering to the subject a therapeutically effective amount of the antibody or an antigen-binding fragment thereof provided herein or the pharmaceutical composition provided herein, optionally in combination with a target antibody that specifically binds to a target antigen on the target cell.

In another aspect, the present disclosure also provides a method of treating, preventing or alleviating a SIRPα related disease disorder or condition in a subject, comprising administering to the subject a therapeutically effective amount of the antibody or an antigen-binding fragment thereof provided herein or the pharmaceutical composition provided herein, optionally in combination with a target antibody that specifically binds to a target antigen on the target cell.

In certain embodiments, the target cell is a CD47 expressing cell.

In certain embodiments, the target cell is a cancer cell, inflammatory cell, and/or a chronically infected cell.

In certain embodiments, the target antigen is tumor antigen, tumor surface antigen, an inflammatory antigen, an antigen of an infectious microorganism.

In certain embodiments, the antibody or an antigen-binding fragment thereof comprises the HCDR1 comprising the sequence of SEQ ID NO: 13, the HCDR2 comprising the sequence of SEQ ID NO: 14 or SEQ ID NO: 17, the HCDR3 comprising the sequence of SEQ ID NO: 15, the LCDR1 comprising the sequence of SEQ ID NO: 10, the LCDR2 comprising the sequence of SEQ ID NO: 11, and the LCDR3 comprising the sequence of SEQ ID NO: 16.

In certain embodiments, the disease, disorder or condition is cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, or arthritis.

In certain embodiments, the cancer is anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, head and neck squamous cell carcinoma, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma (NHL), multiple myeloma, T or B cell lymphoma, GI organ interstitialoma, soft tissue tumor, hepatocellular carcinoma, and adenocarcinoma.

In certain embodiments, the cancer is a CD47-positive cancer.

In certain embodiments, the subject is human.

In certain embodiments, the administration is via oral, nasal, intravenous, subcutaneous, sublingual, or intramuscular administration.

In certain embodiments, the method provided herein further comprises administering a therapeutically effective amount of an additional therapeutic agent.

In certain embodiments, the additional therapeutic agent is selected from the group consisting of a chemotherapeutic agent, an anti-cancer drug, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, cytokines, anti-infectious agent, and anti-inflammatory agent.

In another aspect, the present disclosure also provides a kit comprising the antibody or an antigen-binding fragment thereof provided herein or the pharmaceutical composition provided herein, and a target antibody that binds to a target antigen expressed on the target cell.

In certain embodiments, the target antigen is tumor antigen, tumor surface antigen, or an infectious agent surface antigen.

In certain embodiments, the kit provided herein further comprises an additional therapeutic agent.

In another aspect, the present disclosure also provides a method of modulating SIRPα activity in a SIRPα-positive cell, comprising exposing the SIRPα-positive cell to the antibody or antigen-binding fragment thereof provided herein or the pharmaceutical composition provided herein.

In certain embodiments, the cell is a phagocytic cell.

In another aspect, the present disclosure also provides a method of detecting the presence or amount of SIRPα in a sample, comprising contacting the sample with the antibody or an antigen-binding fragment thereof provided herein, and determining the presence or the amount of SIRPα in the sample.

In another aspect, the present disclosure also provides use of the antibody or an antigen-binding fragment thereof provided herein or the pharmaceutical composition provided herein in the manufacture of a medicament for:
i) treating, preventing or alleviating a SIRPα related disease, disorder or condition in a subject;
ii) inducing phagocytosis of a target cell in a subject;
ii) increasing antibody-dependent cellular phagocytosis (ADCP) effect of a target antibody on a target cell in a subject.

In another aspect, the present disclosure also provides a method of potentiating a target antibody in treating a disease, disorder or condition in a subject, comprising: administering to the subject a therapeutically effective amount of the antibody or an antigen-binding fragment thereof provided herein or the pharmaceutical composition provided herein, in combination with the target antibody, thereby potentiating the target antibody in treating the disease, disorder or condition in the subject.

In certain embodiments, the disease, disorder or condition is immune related disease or disorder, tumors and cancers, autoimmune diseases, or infectious disease.

In certain embodiments, the immune related disease or disorder is selected from the group consisting of systemic lupus erythematosus, acute respiratory distress syndrome (ARDS), vasculitis, myasthenia gravis, idiopathic pulmonary fibrosis, Crohn's Disease, asthma, rheumatoid arthritis, graft versus host disease, a spondyloarthropathy (e.g., ankylosing spondylitis, psoriatic arthritis, isolated acute enteropathic arthritis associated with inflammatory bowel disease, reactive arthritis, Behcet's syndrome, undifferentiated spondyloarthropathy, anterior uveitis, and juvenile idiopathic arthritis.), multiple sclerosis, endometriosis, glomerulonephritis, sepsis, diabetes, acute coronary syndrome, ischemic reperfusion, psoriasis, progressive systemic sclerosis, atherosclerosis, Sjogren's syndrome, scleroderma, or inflammatory autoimmune myositis.

In certain embodiments, the tumors and cancers are solid tumor or hematologic malignancy, optionally selected from the group consisting of non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma, anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma, or the metastases thereof.

### BRIEF DESCFRIPTION OF THE DRAWINGS

FIG. 1 shows FACS binding curves of anti-SIRPα antibodies 025c, 015c, 042c, 059c, hu1H9G4 (FIG. 1A), 071c, 073c (FIG. 1B) against CHOK1-human SIRPα v1 cells and 005c (FIG. 1C) against 293F-human SIRPα v1 cells.

FIG. 2 shows FACS binding curves of anti-SIRPα antibodies 025c, 015c, 042c, 071c, 073c, hu1H9G4 (FIG. 2A), 025c, 059c, 005c, HEFLB (FIG. 2B) against CHOK 1-human SIRPα v2 cells.

FIG. 3 shows FACS binding curves of anti-SIRPα antibodies against CHOK 1-human SIRPB cells (FIG. 3C) and their ELISA binding curves against recombinant proteins of human SIRPβ ECD (FIGS. 3A and 3B) and human SIRPBI ECD (FIGS. 3D and 3E).

FIG. 4 shows FACS binding curves of anti-SIRPα antibodies against 293F-human SIRPγ cells (FIG. 4A and FIG. 4B) and their ELISA binding curves against recombinant protein of cyno SIRPγ ECD (FIG. 4C).

FIG. 5 shows ELISA binding of anti-SIRPα antibodies against recombinant protein of C57BL/6 mouse SIRPα ECD (FIG. 5A) and their FACS binding curves against CHOK 1-cyno SIRPα cells (FIG. 5B and FIG. 5C).

FIG. 6 shows CD47 and SIRPα v1 interaction blocking activity of anti-SIRPα antibodies 025c, 015c, 042c, 059c, 071c, 073c, hu1H9G4 (FIG. 6A), 025c, 005c, 059c (FIG. 6B) as measured by competitive ELISA assay.

FIG. 7 shows CD47 and SIRPα v2 interaction blocking activity of anti-SIRPα antibodies 025c, 059c (FIG. 7A), 025c, 015c, 042c, 071c, 073c, hu1H9G4 (FIG. 7B) as measured by competitive ELISA assay.

FIG. 8 shows the principle of SHP-1 recruitment assay (FIG. 8A) and SHP-1 recruitment blocking activity of anti-SIRPα antibodies as measured by this assay (FIG. 8B).

FIG. 9 shows potential binding epitopes of anti-SIRPα antibodies 025c (FIG. 9A), 042c (FIG. 9B), 073c (FIG. 9C), hu1H9G4 (FIG. 9D), HEFLB (FIG. 9E) as measured by HDX-MS.

FIG. 10 shows phagocytosis of Raji cells (FIG. 10A), DLD1 cells (FIG. 10B), and Raji/PD-L1 cells (FIG. 10C and FIG. 10D) by human macrophages in the presence of the indicated antibodies.

FIG. 11 shows phagocytosis of Raji/PD-L1 cells by human M0 polarized macrophages (FIG. 11A) or human M1 polarized macrophages (FIG. 11B) in the presence of the indicated antibodies.

FIG. 12 shows the results of an in vivo syngeneic mouse colon carcinoma model to assess activity of combination of anti-SIRPα treatment and anti-CLDN18.2 treatment. FIG. 12A shows the weight of each tumor at the end of the study, FIG. 12B shows average tumor volume growth curves for each study group, and FIG. 12C shows individual volume growth curves for each tumor. *p<0.05, **p<0.01, ***p<0.001.

FIG. 13 shows allogeneic dendritic cells stimulated T cell IFNγ secretion (FIG. 13A), proliferation ratios of CD4⁺ T cells (FIG. 13B) and CD8⁺ T cells (FIG. 13C) in the presence of anti-SIRPα antibodies.

FIG. 14 shows FACS binding curves of humanized antibodies against CHOK 1-human SIRPα v1 cells (FIG. 14A), CHOK1-human SIRPα v2 cells (FIG. 14B), CHOK 1-human SIRPB cells (FIG. 14C), and 293F-SIRPγ cells (FIG. 14D).

FIG. 15 shows CD47 and SIRPα interaction blocking activity of humanized antibodies as measured by competitive ELISA assay. (FIG. 15A) human CD47 and human SIRPα v1 interaction blocking, (FIG. 15B) human CD47 and human SIRPα v2 interaction blocking.

FIG. 16 shows CD47 and SIRPα interaction blocking activity of humanized antibodies as measured by competitive FACS assay. (FIG. 16A) human CD47 and human SIRPα v1 interaction blocking, (FIG. 16B) human CD47 and human SIRPα v2 interaction blocking.

FIG. 17 shows SHP-1 recruitment blocking activity of humanized antibodies as measured by SHP-1 recruitment assay.

FIG. 18 shows phagocytosis of Raji/PD-L1 cells by human macrophages in the presence of the indicated antibodies. (FIG. 18A, FIG. 18C and FIG. 18E) phagocytosis of Raji/PD-L1 cells by human macrophages from SIRPA homozygous v1/v1 (A), SIRPA homozygous v2/v2 (C) or SIRPA heterozygous v1/v2 (E) donor in the presence of anti-SIRPα antibodies plus anti-PD-L1 antibody, (FIG. 18B and FIG. 18D) phagocytosis of Raji/PD-L1 cells by human macrophages SIRPA homozygous v1/v1 (B) or SIRPA homozygous v2/v2 (D) donor in the presence of anti-SIRPα antibodies plus Rituximab.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273 (4), 927 (1997); Chothia, C. et al., J Mol Biol. December 5; 186 (3): 651-63 (1985); Chothia, C. and Lesk, A. M., J. Mol. Biol., 196,901 (1987); Chothia, C. et al., Nature. December 21-28; 342 (6252): 877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27:55-77 (2003); Marie-Paule Lefranc et al., Immunome Research, 1 (3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody (e.g. of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85: 5879 (1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two VH domains and no light chains (Riechmann L. and Muyldermans S., J Immunol Methods. December 10; 231 (1-2): 25-38 (1999); Muyldermans S., J Biotechnol. June; 74 (4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363 (6428): 446-8 (1993); Nguyen V K. et al. Immunogenetics. April; 54 (1): 39-47 (2002); Nguyen V K. et al. Immunology. May; 109 (1): 93-101 (2003)). The variable domain of a heavy chain antibody (VHH domain) represents the smallest known antigen-binding unit generated by adaptive immune responses (Koch-Nolte F. et al., FASEB J. November; 21 (13): 3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a VH domain connected to a VL domain in the same polypeptide chain (VH-VL or VL-VH) (see, e.g. Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more VH domains are covalently joined with a peptide linker to create a bivalent or multivalent domain antibody. The two VH domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with VLI-V_{H2} (also linked by a peptide linker) such that VHI and VLI coordinate and V_{H2} and V_{L2} coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g. a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (K_{off}/kₒₙ) when the binding between the antigen and antigen-binding molecule reaches equilibrium. K_{D} may be determined by using any conventional method known in the art, including but are not limited to, surface plasmon resonance method, microscale thermophoresis method, HPLC-MS method and flow cytometry (such as FACS) method. A K_{D} value of ≤10⁻⁶ M (e.g. ≤5×10⁻⁷M, ≤2×10⁻⁷M, ≤10⁻⁷M, ≤5×10⁻8 M, ≤2×10⁻⁸ M, ≤10⁻⁸ M, ≤5×10⁻⁹M, ≤4×10⁻⁹M, ≤3×10⁻⁹M, ≤2×10⁻⁹ M, or ≤10⁻⁹ M) can indicate specific binding between an antibody or antigen binding fragments thereof and SIRPα (e.g. human SIRPα).

The ability to “compete for binding to human SIRPα” as used herein refers to the ability of a first antibody or antigen-binding fragment to inhibit the binding interaction between human SIRPα and a second anti-SIRPα antibody to any detectable degree. In certain embodiments, an antibody or antigen-binding fragment that compete for binding to human SIRPα inhibits the binding interaction between human SIRPα and a second anti-SIRPα antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e. including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Name of Amino Acid | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among amino acid residues with neutral hydrophilic side chains (e.g. Cys, Ser, Thr, Asn and Gln), among amino acid residues with acidic side chains (e.g. Asp, Glu), among amino acid residues with basic side chains (e.g. His, Lys, and Arg), or among amino acid residues with aromatic side chains (e.g. Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementary strand) or amino acid sequences that have sequence identity of at least 60% (e.g. at least 65%, 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23 (21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or an antigen-binding fragment thereof, at least one promoter (e.g. SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a SIRPα related disease, or refer to identification of a subject with a SIRPα related disease who may benefit from a particular treatment regimen. In some embodiments, diagnosis contains the identification of abnormal amount or activity of SIRPα. In some embodiments, diagnosis refers to the identification of a cancer or an autoimmune disease in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject.

“SIRPα” as used herein, refers to a regulatory membrane glycoprotein from signal regulatory protein (SIRP) family expressed mainly by myeloid cells, dendritic cells and also by stem cells or neurons. The structure of SIRPα includes an extracellular domain and a cytoplasmic domain. The extracellular domain of SIRPα consists of a membrane-distal Ig variable-like (IgV) fold, and two membrane-proximal Ig constant-like (IgC) folds. The IgV domain of SIRPα is responsible for the binding of the extracellular Ig-domain of CD47. In certain embodiments, the SIRPα is human SIRPα. The gene coding for human SIRPα is a polymorphic gene and several variants were described in human population. The most common protein variants are SIRPα v1 and SIRPα v2 (accession numbers NP_542970 (P78324) and CAA71403). SIRPα as used herein may be from other animal species, such as from mouse, and cynomolgus, among others. Exemplary sequence of Mus musculus (mouse) SIRPα protein is disclosed in NCBI Ref Seq No. NP_031573, or BAA20376.1, or BAA13521.1. Exemplary sequence of Cynomolgus (monkey) SIRPα protein is disclosed in NCBI Ref Seq No. NP_001271679.

In addition to SIRPα, the SIRPs family also comprise several other transmembrane glycoproteins, including, SIRPB and SIRPγ. Each member of the SIRPs family contains 3 similar extracellular Ig-like domains with distinct transmembrane and cytoplasmic domains. “SIRPB”, encoded by SIRP beta gene, generates a positive signal by intracellular signaling of its cytoplasmic tail through its association with a transmembrane protein called DNAX activation protein 12 or DAP12. The cytoplasmic tail of DAP12 possesses immunoreceptor tyrosine-based activation motifs (ITAMs) that link SIRPβ1 to activation machinery. “SIRPγ”, also named as SIRPg, is encoded by the SIRPG gene, and is highly homologous in the extracellular Ig domains to SIRPα and SIRPB, but the cytoplasmic tail of SIRPγ is distinct. SIRPγ was also shown to bind to CD47 but with a lower affinity than SIRPα.

The term “anti-SIRPα antibody” refers to an antibody that is capable of specific binding to SIRPα (e.g. human or monkey SIRPα). The term “anti-human SIRPα antibody” refers to an antibody that is capable of specific binding to human SIRPα.

A “SIRPα related” disease, disorder or condition as used herein refers to any disease or condition caused by, exacerbated by, or otherwise linked to increased or decreased expression or activities of SIRPα. In some embodiments, the SIRPα related disease, disorder or condition is an immune-related disorder, such as, for example, an autoimmune disease. In some embodiments, the SIRPα related disease, disorder or condition is a disorder related to excessive cell proliferation, such as, for example, cancer. In certain embodiments, the SIRPα related disease or condition is characterized in expressing or over-expressing of SIRPα gene and/or SIRPα signature genes. In certain embodiments, the SIRPα related disease or condition is characterized in expressing or over-expressing of CD47.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “SIRPα-positive cell” as used herein refer to a cell (e.g. a phagocytic cell) that expresses SIRPα on the surface of the cell. In some embodiments, a “SIRPα-positive cell” may also express SIRPB or SIRPγ on the surface of the cell.

### Anti-SIRPα Antibodies

The present disclosure provides anti-SIRPα antibodies and antigen-binding fragments thereof. The anti-SIRPα antibodies and antigen-binding fragments provided herein are capable of specific binding to SIRPα.

In certain embodiments, the antibodies and the antigen-binding fragments thereof provided herein specifically bind to human SIRPα at an K_{D} value of no more than 10⁻⁷ M, no more than 8×10⁻⁸ M, no more than 5×10⁻⁸ M, no more than 2×10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, or no more than 6×10⁻¹⁰ M, or no more than 5×10⁻¹⁰ M, or no more than 4×10⁻¹⁰ M using Bio-Layer Interferometry technology (Octet system). Octet system is based on bio-layer interferometry (BLI) technology, see, for example, Sultana A. et al., Current protocols in protein science, 2 Feb. 2015, 79:19.25.1-19.25.26. In certain embodiments, the K_{D} value is measured by the method as described in Example 5.2.5 of the present disclosure.

Binding of the antibodies or the antigen-binding fragments thereof provided herein to human SIRPα can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), flow cytometry assay, and other binding assay. In certain embodiments, the antibodies and the antigen-binding fragments thereof provided herein specifically bind to human SIRPα v1 or human SIRPα v2 at an EC₅₀ (i.e. 50% binding concentration) of no more than 0.5 nM, no more than 0.2 nM, no more than 0.1 nM, no more than 0.09 nM, no more than 0.08 nM, no more than 0.07 nM, no more than 0.06 nM or no more than 0.05 nM as measured by enzyme-linked immunosorbent assay (ELISA).

In certain embodiments, the antibodies and the antigen-binding fragments thereof provided herein specifically bind to human SIRPα vlat an EC₅₀ of no more than 4 nM (e.g. no more than 3 nM, no more than 2 nM, no more than 1.5 nM, no more than 1.0 nM) as measured by FACS assay.

In certain embodiments, the antibodies and the antigen-binding fragments thereof provided herein specifically bind to human SIRPα v2 at an EC₅₀ of no more than 12.1 nM (e.g. no more than 6 nM, no more than 5 nM, no more than 4 nM, no more than 3 nM, no more than 2 nM, no more than 1 nM, no more than 0.9 nM, no more than 0.8 nM, no more than 0.7 nM) as measured by FACS assay.

In certain embodiments, the antibodies and antigen-binding fragments thereof provided herein has no specific binding to mouse SIRPα. An antibody or antigen-binding fragment thereof that with “no specific binding” to mouse SIRPα is one that exhibits no detectable binding to mouse SIRPα or exhibits a binding to mouse SIRPα at a level comparable to that a control antibody under equivalent assay conditions. A control antibody can be any antibody that is known not to bind to mouse SIRPα.

In certain embodiments, the antibodies and antigen-binding fragments thereof provided herein specifically bind to SIRPBat an EC₅₀ of no more than 40 nM (e.g. no more than 30 nM, no more than 1 nM, no more than 0.9 nM, no more than 0.8 nM, no more than 0.7 nM, no more than 0.4 nM) as measured by FACS assay.

In certain embodiments, the antibodies and antigen-binding fragments thereof provided herein specifically bind to SIRPB ECD at an EC₅₀ of no more than 3 nM (e.g., no more than 2 nM, no more than 0.9 nM, no more than 0.8 nM, no more than 0.7 nM, no more than 0.5 nM, no more than 0.4 nM, no more than 0.3 nM, no more than 0.1 nM, no more than 0.05 nM,) as measured by ELISA assay.

In certain embodiments, the antibodies and antigen-binding fragments thereof provided herein bind to SIRPγ at an EC₅₀ of no more than 80 nM (e.g. no more than 50 nM, no more than 40 nM, no more than 20 nM, no more than 10 nM, no more than 1 nM, no more than 0.3 nM) as measured by FACS assay.

In certain embodiments, the anti-SIRPα antibody or an antigen-binding fragment thereof provided herein are capable of completely blocking interaction between SIRP-alpha and CD47. By “completely block interaction” between two interacting molecules, it is meant that an antibody is capable of inhibiting at least 80% binding between the two interacting molecules, or capable of inhibiting at least 50% signal transduction induced by interaction of the two molecules. The signal transduction induced by interaction between SIRP-alpha and CD47 can be characterized by SHP1 recruitment to intracellular portion (e.g. C-terminal tail) of SIRP-alpha.

In certain embodiments, the anti-SIRPα antibody or an antigen-binding fragment thereof provided herein capable of completely blocking interaction between SIRP-alpha v1 and CD47. In certain embodiments, the anti-SIRPα antibody or an antigen-binding fragment thereof provided herein are capable of blocking at least 85%, 90%, 91%, 92%, 93%, 94%, 95%, 96%, 97% of the binding between SIRP-alpha v1 and CD47 as measured by competitive ELISA assay. In certain embodiments, the anti-SIRPα antibody or an antigen-binding fragment thereof provided herein are capable of blocking at least 97% or at least 98% of the binding between SIRP-alpha v1 and CD47 as measured by competitive FACS assay. In certain embodiments, the anti-SIRPα antibody or an antigen-binding fragment thereof provided herein are capable of blocking interaction between SIRP-alpha v1 and CD47 at an IC₅₀ of no more than 4 nM (or no more than 3 nM), as measured by competitive ELISA assay or at an IC₅₀ of no more than 0.6 nM (or no more than 0.5 nM), as measured by competitive FACS assay.

In certain embodiments, the anti-SIRPα antibody or an antigen-binding fragment thereof provided herein capable of completely blocking interaction between SIRP-alpha v2 and CD47. In certain embodiments, the anti-SIRPα antibody or an antigen-binding fragment thereof provided herein are capable of blocking at least 80%, 85%, 90%, 95%, 96%, 97% or 98% of the binding between SIRP-alpha v2 and CD47 as measured by competitive ELISA assay. In certain embodiments, the anti-SIRPαantibody or an antigen-binding fragment thereof provided herein are capable of blocking at least 98% or at least 99% of the binding between SIRP-alpha v2 and CD47 as measured by competitive FACS assay. In certain embodiments, the anti-SIRPα antibody or an antigen-binding fragment thereof provided herein capable of blocking interaction between SIRP-alpha v2 and CD47 at an IC₅₀ of no more than 55 nM (or no more than 6 nM, no more than 5 nM, no more than 3 nM or no more than 2 nM), as measured by competitive ELISA assay or at an IC₅₀ of no more than 3 nM (or no more than 2 nM), as measured by competitive FACS assay.

In certain embodiments, the anti-SIRPα antibody or an antigen-binding fragment thereof provided herein are capable of blocking the binding at least 50%, 60%, 70%, or 80% of signal transduction induced by interaction of SIRP-alpha and CD47.

In certain embodiments, an antibody may block signal transduction induced by interaction between SIRP-alpha and CD47, but does not significantly block binding between the SIRP-alpha and CD47. In other words, while SIRP-alpha and CD47 can bind to each other in the presence of such an anti-SIRP-alpha antibody, they are rendered less effective in signal transduction.

In certain embodiments, the anti-SIRPα antibody or an antigen-binding fragment thereof provided herein have no significant inhibition on IFNγ secretion by T cells, CD4⁺ T cell proliferation or CD8⁺ T cell proliferation. It has been reported that adhesion of human T cells to antigen-presenting cells through SIRPγ-CD47 interaction co-stimulates T cell proliferation. T cell proliferation can be determined using methods known in the art, for example, by T cell proliferation assay such as those described in Example 4.2.9 of the present disclosure, for example, by using CellTrace Violet (Life Technologies) labelling to determine proliferation population. As shown in the present disclosure, regardless of binding activity to human SIRPγ, the antibodies or antigen-binding fragment thereof provided herein do not significantly reduce proliferation of CD4⁺ T cells or CD8⁺ T cells or impact IFNγ secretion.

In certain embodiments, the anti-SIRPα antibody or an antigen-binding fragment thereof provided herein show no more than 50% (or no more than 40%, no more than 30%, no more than 20%, or no more than 10%) inhibition on the IFNγ secretion by T cells, CD4⁺ T cell proliferation or CD8⁺ T cell proliferation, relative to a control level obtained with a control antibody (e.g. an antibody that is known not to bind to SIRPα and not to affect T cell proliferation). In certain embodiments, the anti-SIRPα antibody or an antigen-binding fragment thereof provided herein show no detectable inhibition on IFNγ secretion by T cells, CD4⁺ T cell proliferation or CD8⁺ T cell proliferation.

In certain embodiments, the anti-SIRPα antibody or an antigen-binding fragment thereof provided herein as a single agent do not induce phagocytosis of certain CD47-expressing cell such as Raji cell.

In certain embodiments, the anti-SIRPα antibody or an antigen-binding fragment thereof provided herein is capable of increasing antibody-dependent cellular phagocytosis (ADCP) effect of a target antibody. In certain embodiments, the target antibody binds to a target antigen expressed on the target cell, and the ADCP effects of the target antibody on the target cell is increased. In certain embodiments, the target cell also expresses CD47.

In certain embodiments, the anti-SIRPα antibody or an antigen-binding fragment thereof provided herein are capable of binding to an epitope comprising an amino acid sequence of YNQKEGHFPRVTTVSDL (SEQ ID NO: 36).

In certain embodiments, the anti-SIRPα antibody or an antigen-binding fragment thereof provided herein are capable of binding to an epitope comprising an amino acid sequence of SGAGTEL (SEQ ID NO: 72), and/or TNVDPVGESVS (SEQ ID NO: 87).

In certain embodiments, the anti-SIRPα antibody or an antigen-binding fragment thereof provided herein are capable of binding to an epitope comprising an amino acid sequence of TNVDPVGESVSY (SEQ ID NO: 90).

### Illustrative Anti-SIRPα Antibodies

In certain embodiments, the present disclosure provides anti-SIRPα antibodies and antigen-binding fragments thereof comprising one or more (e.g. 1, 2, 3, 4, 5, or 6) CDRs sequences of antibodies 005, 015, 025, 042, 071, 073 and/or 059. In certain embodiments, the present disclosure provides chimeric antibodies, humanized antibodies, antibody derivatives and antibody variants of antibodies 005, 015, 025, 042, 071, 073 and/or 059.

Antibody “005” and “005c” as used herein respectively refers to a monoclonal hybridoma antibody and chimeric antibody comprising a heavy chain variable region having the amino acid sequence of SEQ ID NO: 49, and a light chain variable region having the amino acid sequence of SEQ ID NO: 50.

Antibody “015” and “015c” as used herein respectively refers to a monoclonal hybridoma antibody and chimeric antibody comprising a heavy chain variable region having the amino acid sequence of SEQ ID NO: 51, and a light chain variable region having the amino acid sequence of SEQ ID NO: 52.

Antibody “025” and “025c” as used herein respectively refers to a monoclonal hybridoma antibody and chimeric antibody comprising a heavy chain variable region having the sequence of SEQ ID NO: 53, and a light chain variable region having the sequence of SEQ ID NO: 54.

Antibody “042” and “042c” as used herein respectively refers to a monoclonal hybridoma antibody and chimeric antibody comprising a heavy chain variable region having the sequence of SEQ ID NO: 55, and a light chain variable region having the sequence of SEQ ID NO: 56.

Antibody “059” and “059c” as used herein respectively refers to a monoclonal hybridoma antibody and chimeric antibody comprising a heavy chain variable region having the sequence of SEQ ID NO: 57, and a light chain variable region having the sequence of SEQ ID NO: 58.

Antibody “071” and “071c” as used herein respectively refers to a monoclonal hybridoma antibody and chimeric antibody comprising a heavy chain variable region having the sequence of SEQ ID NO: 59, and a light chain variable region having the sequence of SEQ ID NO: 60.

Antibody “073” and “073c” as used herein respectively refers to a monoclonal hybridoma antibody and chimeric antibody comprising a heavy chain variable region having the sequence of SEQ ID NO: 61, and a light chain variable region having the sequence of SEQ ID NO: 62.

Table 1 below shows the CDR amino acid sequences of antibodies 005, 015, 025, 042, 071, 073, 059, 005c, 015c, 025c, 042c, 059c, 071c and 073. The CDR boundaries in Table 1 were defined or identified by the convention of Kabat, although a skilled person in the art can appreciate that CDRs can also be defined using other conventions such as IMGT, Chothia, or Al-Lazikani, or can be defined in a mixed way using two or more conventions. Table 2 below shows the heavy chain and light chain variable region amino acid sequences of antibodies 005, 015, 025, 042, 071, 073, 059, 005c, 015c, 025c, 042c, 059c, 071c and 073.

TABLE 1
CDR amino acid sequences of 7 antibodies

| Anti- | | | | | | |
|---|---|---|---|---|---|---|
| body | | | | | | |
| ID | HCDR1 | HCDR2 | HCDR3 | LCDR1 | LCDR2 | LCDR3 |
| 005/ | SEQ ID | SEQ ID | SEQ ID | SEQ ID | SEQ ID | SEQ ID |
| 005c | NO: 1 | NO: 2 | NO: 3 | NO: 4 | NO: 5 | NO: 6 |
| | DYYMS | FIKNEANG | YDYYGSNY | KASQNVR | LASKRHT | LQHWIHP |
| | | YTTESSAS | NWYFDA | TAVA | | LT |
| | | VKG | | | | |
| 015/ | SEQ ID | SEQ ID | SEQ ID | SEQ ID | SEQ ID | SEQ ID |
| 015c | NO: 7 | NO: 8 | NO: 9 | NO: 10 | NO: 11 | NO: 12 |
| | AYYMH | RIDPEDGE | GSYEY | SASSSVSSS | STSNLAS | YQWSSYP |
| | | SKYAPKFQ | | YLY | | YT |
| | | G | | | | |
| 025/ | SEQ ID | SEQ ID | SEQ ID | SEQ ID | SEQ ID | SEQ ID |
| 025c | NO: 13 | NO: 14 | NO: 15 | NO: 10 | NO: 11 | NO: 16 |
| | DYYMH | RIDPEDGE | GLAY | SASSSVSSS | STSNLAS | HQWSSYP |
| | | TKYAPKFQ | | YLY | | YT |
| | | G | | | | |
| | | SEQ ID | | | | |
| | | NO: 17 | | | | |
| | | RIDPEDAE | | | | |
| | | TKYAPKFQ | | | | |
| | | G | | | | |
| | SEQ ID | SEQ ID | SEQ ID | | | SEQ ID |
| | NO: 18 | NO: 19 | NO: 20 | | | NO: 21 |
| | X₁YYMH | RIDPEDX₂E | GX₁₈X₄X₅Y | | | X₆QWSSY |
| | | X₃KYAPKF | | | | PYT |
| | | QG | | | | |
| 042/ | SEQ ID | SEQ ID | SEQ ID | SEQ ID | SEQ ID | SEQ ID |
| 042c | NO: 22 | NO: 23 | NO: 24 | NO: 25 | NO: 26 | NO: 27 |
| | TYGMS | WINTYSG | DPHSYGN | KASQNVGI | SASNRYT | QQYSSYP |
| | | VSTCADDF | SPAWFPY | SVA | | LT |
| | | KG | | | | |
| 071/ | SEQ ID | SEQ ID | SEQ ID | SEQ ID | SEQ ID | SEQ ID |
| 071c | NO: 22 | NO: 28 | NO: 29 | NO: 30 | NO: 31 | NO: 32 |
| | TYGMS | WINTYSG | DPHYYGTS | KASQIVGI | SASNRFT | QQYSTYP |
| | | VPTYADDF | PAWFAY | AVA | | FT |
| | | QG | | | | |
| 073/ | SEQ ID | SEQ ID | SEQ ID | SEQ ID | SEQ ID | SEQ ID |
| 073c | NO: 22 | NO: 33 | NO: 34 | NO: 35 | NO: 26 | NO: 37 |
| | TYGMS | WINTYSG | DPHYYGSS | EASQIVGI | SASNRYT | QQYSAYP |
| | | VPTYADDF | PAWFVY | AVA | | FT |
| | | KG | | | | |
| | | SEQ ID | SEQ ID | SEQ ID | SEQ ID | SEQ ID |
| | | NO: 38 | NO: 39 | NO: 40 | NO: 41 | NO: 42 |
| | | WINTYSG | DPHX₉YG | X₁₂ASQX₁₃ | SASNRX₁₅ | QQYSX₁₆ |
| | | VX₁₉TX₇A | X₁₀SPAWF | VGIX₁₄V | T | YPX₁₇T |
| | | DDFX₈G | X₁₁Y | A | | |
| 059/ | SEQ ID | SEQ ID | SEQ ID | SEQ ID | SEQ ID | SEQ ID |
| 059c | NO: 43 | NO: 44 | NO: 45 | NO: 46 | NO: 47 | NO: 48 |
| | EYVLS | EIYPGTITT | FYDYDGG | SASSSVSSS | GTSNLAS | QQWSGY |
| | | YYNEKFKG | WFAY | DLH | | PWT |

X₁ is A or D; X₂ is G or A; X₃ is T or S; X₄ is L or Y; X₅ is E or A; X₆ is Y or H ; X₇ is Y or C; X₈ is K or Q ; X₉ is Y or S; X₁₀ is N or T or S; X₁₁ is P or A or V; X₁₂ is E or K; X₁₃ is N or I; X₁₄ is S or A; X₁₅ is Y or F; X₁₆ is S or T or A; X₁₇ is F or L; X₁₈ is S or absent; X₁₉ is S or P.

TABLE 2
Variable region amino acid sequences of 7 antibodies

| Antibody | | |
|---|---|---|
| ID | Heavy Chain Variable Region | Light Chain Variable Region |
| 005/005c | SEQ ID NO: 49 | SEQ ID NO: 50 |
| | EVKLVESGGGLVQPGGSLSLSCAA | DIVMTQSQKFMSPSVGDRVSITCKAS |
| | SGFTFTDYYMSWVRQPPGKALEW | QNVRTAVAWYQQKPGQSPKVLIHLA |
| | LGFIKNEANGYTTESSASVKGRFTIS | SKRHTGVPDRFTGSGSGTDFTLTISNV |
| | RDNSQSILYLQMNALRAEDSATYY | QSEDLADYFCLQHWIHPLTFGAGTKL |
| | CARYDYYGSNYNWYFDAWGTGT | ELK |
| | TVTVSS | |
| 015/015c | SEQ ID NO: 51 | SEQ ID NO: 52 |
| | EVQLQQSGVEVVQPGASVKLSCT | QIVLTQSPAIMSASPGEKVTLTCSASS |
| | ASGFNIEAYYMHWVKORTEQGLE | SVSSSYLYWYQQKPGSSPKLWIYSTSN |
| | WIGRIDPEDGESKYAPKFOGKAT | LASGVPPRFSGSGSGTSYSLTISSMQA |
| | MTADTSSSTAYLQLSSLTSDDTAVY | EDAASYFCYQWSSYPYTFGGGTKLEIK |
| | YCVRGSYEYWGQGTTLTVSS | |
| 025/025c | SEQ ID NO: 53 | SEQ ID NO: 54 |
| | EVQLQQSGAELVKPGASVKLSCTA | QIVLTQSPAIMSASPGEKVTLTCSASS |
| | SGFNIKDYYMHWVKQRTEQGLE | SVSSSYLYWYQQKPGSSPKLWIYSTSN |
| | WIGRIDPEDGETKYAPKFQGKATIT | LASGVPARFSGSGSGTSYSLTISSMEA |
| | ADTSSNTAYLQLSSLTSEDTAVYYC | EDAASYFCHQWSSYPYTFGGGTKLEI |
| | DRGLAYWGQGTLVTVSA | K |
| 042/042c | SEQ ID NO: 55 | SEQ ID NO: 56 |
| | QIQLVQSGPELKKPGETVKISCRAS | DIVMTQSQKFMSTTIRDRVSITCKAS |
| | GYTFTTYGMSWVKQAPGKGLRW | QNVGISVAWYQQKSGQSPKLLIYSAS |
| | MGWINTYSGVSTCADDFKGRFAF | NRYTGVPDRFTGSGSGTDFTLTISNM |
| | SLETSATTAYLQIHNLTNEDTATYF | QSEDLADYFCQQYSSYPLTFGSGTKLA |
| | CARDPHSYGNSPAWFPYWGQGT | IK |
| | LVTVSA | |
| 059/059c | SEQ ID NO: 57 | SEQ ID NO: 58 |
| | QVQLQQSGPELVKPGASVKMSCK | ENVLTQSPEKMAVSLGQKVTMTCSA |
| | ASGYTFSEYVLSWVKORTGQGLE | SSSVSSSDLHWYQQKSGASPKPLIHG |
| | WIGEIYPGTITTYYNEKFKGKATLTA | TSNLASGVPARFSGSGSGTSYSLTISSV |
| | DKSSNTAYIQLTSLTSEDSAVYFCG | EAEDAATYYCQQWSGYPWTFGGGT |
| | RFYDYDGGWFAYWGQGTLLTVSA | NLEIK |
| 071/071c | SEQ ID NO: 59 | SEQ ID NO: 60 |
| | QIQLVQSGPELKKPGETVKISCKAS | DIVMTQSQKFMSTTIGDRVIITCKASQ |
| | GYTFTTYGMSWVKQAPGKGLKW | IVGIAVAWYQQKPGQSPKLLIYSASNR |
| | MVWINTYSGVPTYADDFQGRFAF | FTGVPDRFTGSGSGTDFTLTISNMQS |
| | SLETSASTSYLQINNLRNEDTATYFC | EDLADYFCQQYSTYPFTFGSGTKLEIK |
| | ARDPHYYGTSPAWFAYWGQGTL | |
| | VTVSA | |
| 073/073c | SEQ ID NO: 61 | SEQ ID NO: 62 |
| | QIQLVQSGPELKKPGETVKISCKAS | DIVMTQSQKFMSTTIGDRVSITCEAS |
| | GYTFTTYGMSWVKQAPGKGLKW | QIVGIAVAWYQQKPGQSPKLLIYSAS |
| | MVWINTYSGVPTYADDFKGRFAF | NRYTGVPDRFTGSGSGTDFTLTISNM |
| | SLETSASTSYLQINNLKNEDTATYFC | QSEDLANYFCQQYSAYPFTFGSGTKL |
| | ARDPHYYGSSPAWFVYWGQGTL | EVK |
| | VTVSA | |

Given that each of antibodies 005, 015, 025, 042, 059, 071, 073, 005c, 015c, 025c, 042c, 059c, 071c and 073 can bind to SIRPα and that antigen-binding specificity is provided primarily by the CDR1, CDR2 and CDR3 regions, the HCDR1, HCDR2 and HCDR3 sequences and LCDR1, LCDR2 and LCDR3 sequences of antibodies 005, 015, 025, 042, 059, 071, 073, 005c, 015c, 025c, 042c, 059c, 071c and 073 can be “mixed and matched” (i.e., CDRs from different antibodies can be mixed and matched, but each antibody must contain a HCDR1, HCDR2 and HCDR3 and a LCDR1, LCDR2 and LCDR3) to create anti-SIRPα binding molecules of the present disclosure. SIRPα binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence(s). Likewise, when VL CDR sequences are mixed and matched, the LCDR1, LCDR2 and/or LCDR3 sequence from a particular VL sequence preferably is replaced with a structurally similar CDR sequence(s). It will be readily apparent to a person skilled in the art that novel VH and VL sequences can be created by substituting one or more VH and/or VL CDR region sequences with structurally similar sequences from the CDR sequences disclosed herein for monoclonal hybridoma antibodies 005, 015, 025, 042, 059, 071 and 073 or for chimeric antibodies 005c, 015c, 025c, 042c, 059c, 071c and 073c.

In certain embodiments, the present disclosure provides anti-SIRPα antibodies and antigen-binding fragments thereof comprising HCDR1 comprising the sequence selected from the group consisting of SEQ ID NOs: 1, 7, 13, 18, 22 and 43, HCDR2 comprising the sequence selected from the group consisting of SEQ ID NOs: 2, 8, 14, 17, 19, 23, 28, 33, 38 and 44, and HCDR3 comprising the sequence selected from the group consisting of SEQ ID NOs: 3, 9, 15, 20, 24, 29, 34, 39 and 45, and/or LCDR1 comprising the sequence selected from the group consisting of SEQ ID NOS: 4, 10, 25, 30, 35, 40 and 46, LCDR2 comprising the sequence selected from the group consisting of SEQ ID NOs: 5, 11, 26, 31, 41 and 47, and LCDR3 comprising the sequence selected from the group consisting of SEQ ID NOs: 6, 12, 16, 21, 27, 32, 37, 42 and 48.

In certain embodiments, the present disclosure provides anti-SIRPα antibodies and antigen-binding fragments thereof comprising the HCDR1 comprises an amino acid sequence of X₁YYMH (SEQ ID NO: 18); the HCDR2 comprises an amino acid sequence of RIDPEDX₂EX₃KYAPKFQG (SEQ ID NO: 19); the HCDR3 comprises an amino acid sequence of GX₁₈X₄X₅Y (SEQ ID NO: 20); the LCDR1 comprises an amino acid sequence of SEQ ID NO: 10; the LCDR2 comprises an amino acid sequence of SEQ ID NO: 11, and the LCDR3 comprises an amino acid sequence of X₆QWSSYPYT (SEQ ID NO: 21), wherein X₁ is A or D; X₂ is G or A; X₃ is T or S; X₄ is L or Y; X₅ is E or A; X₆ is Y or H; and X₁₈ is S or absent.

In certain embodiments, the present disclosure provides anti-SIRPα antibodies and antigen-binding fragments thereof comprising HCDR1 comprising the sequence selected from the group consisting of SEQ ID NOs: 7 and 13; and/or HCDR2 comprising the sequence selected from the group consisting of SEQ ID NOs: 8, 14, and 17; and/or HCDR3 comprising the sequence selected from the group consisting of SEQ ID NOs: 9 and 15; and/or LCDR1 comprising the sequence of SEQ ID NO: 10; and/or LCDR2 comprising the sequence of SEQ ID NO: 11; and/or LCDR3 comprising the sequence selected from the group consisting of SEQ ID NOs: 12 and 16.

In certain embodiments, the present disclosure provides anti-SIRPα antibodies and antigen-binding fragments thereof comprising the HCDR1 comprises an amino acid sequence of SEQ ID NO: 22; the HCDR2 comprises an amino acid sequence of WINTYSGVX₁₉TX₇ADDFX&G (SEQ ID NO: 38); the HCDR3 comprises an amino acid sequence of DPHX, YGX₁₀SPAWFX₁₁Y (SEQ ID NO: 39); the LCDR1 comprises an amino acid sequence of X₁₂ASQX₁₃ VGIX₁₄VA (SEQ ID NO: 40); the LCDR2 comprises an amino acid sequence of SASNRX₁ST (SEQ ID NO: 41); and the LCDR3 comprises an amino acid sequence of QQYSX 16 YPX₁₇T (SEQ ID NO: 42), wherein X₇ is Y or C; X₈ is K or Q; X₉ is Y or S; X₁₀ is N or T or S; X₁₁ is P or A or V; X₁₂ is E or K; X₁₃ is N or I; X₁₄ is S or A; Xis is Y or F; X₁₆ is S or T or A; X₁₇ is F or L; and X₁₉ is S or P.

In certain embodiments, the present disclosure provides anti-SIRPα antibodies and antigen-binding fragments thereof comprising HCDR1 comprising the sequence of SEQ ID NO: 22; and/or HCDR2 comprising the sequence selected from the group consisting of SEQ ID NOs: 23, 28, and 33; and/or HCDR3 comprising the sequence selected from the group consisting of SEQ ID NOs: 24, 29, and 34; and/or LCDR1 comprising the sequence of SEQ ID NO: 25, 30, and 35; and/or LCDR2 comprising selected from the group consisting of SEQ ID NOs: 31, and 26; and/or LCDR3 comprising the sequence selected from the group consisting of SEQ ID NOs: 27, 32 and 37.

In certain embodiments, the present disclosure provides anti-SIRPα antibodies and antigen-binding fragments thereof comprising a HCDR1 comprising the sequence of SEQ ID NO: 1, a HCDR2 comprising the sequence of SEQ ID NO: 2, a HCDR3 comprising the sequence of SEQ ID NO: 3, a LCDR1 comprising the sequence of SEQ ID NO: 4, a LCDR2 comprising the sequence of SEQ ID NO: 5, and a LCDR3 comprising the sequence of SEQ ID NO: 6.

In certain embodiments, the present disclosure provides anti-SIRPα antibodies and antigen-binding fragments thereof comprising a HCDR1 comprising the sequence of SEQ ID NO: 7, a HCDR2 comprising the sequence of SEQ ID NO: 8, a HCDR3 comprising the sequence of SEQ ID NO: 9, a LCDR1 comprising the sequence of SEQ ID NO: 10, a LCDR2 comprising the sequence of SEQ ID NO: 11, and a LCDR3 comprising the sequence of SEQ ID NO: 12.

In certain embodiments, the present disclosure provides anti-SIRPα antibodies and antigen-binding fragments thereof comprising a HCDR1 comprising the sequence of SEQ ID NO: 13, a HCDR2 comprising the sequence of SEQ ID NO: 14 or 17, a HCDR3 comprising the sequence of SEQ ID NO: 15, a LCDR1 comprising the sequence of SEQ ID NO: 10, a LCDR2 comprising the sequence of SEQ ID NO: 11, and a LCDR3 comprising the sequence of SEQ ID NO: 16.

In certain embodiments, the present disclosure provides anti-SIRPα antibodies and antigen-binding fragments thereof comprising a HCDR1 comprising the sequence of SEQ ID NO: 22, a HCDR2 comprising the sequence of SEQ ID NO: 23, a HCDR3 comprising the sequence of SEQ ID NO: 24, a LCDR1 comprising the sequence of SEQ ID NO: 25, a LCDR2 comprising the sequence of SEQ ID NO: 26, and a LCDR3 comprising the sequence of SEQ ID NO: 27.

In certain embodiments, the present disclosure provides anti-SIRPα antibodies and antigen-binding fragments thereof comprising a HCDR1 comprising the sequence of SEQ ID NO: 22, a HCDR2 comprising the sequence of SEQ ID NO: 28, a HCDR3 comprising the sequence of SEQ ID NO: 29, a LCDR1 comprising the sequence of SEQ ID NO: 30, a LCDR2 comprising the sequence of SEQ ID NO: 31, and a LCDR3 comprising the sequence of SEQ ID NO: 32.

In certain embodiments, the present disclosure provides anti-SIRPα antibodies and antigen-binding fragments thereof comprising a HCDR1 comprising the sequence of SEQ ID NO: 22, a HCDR2 comprising the sequence of SEQ ID NO: 33, a HCDR3 comprising the sequence of SEQ ID NO: 34, a LCDR1 comprising the sequence of SEQ ID NO: 35, a LCDR2 comprising the sequence of SEQ ID NO: 26, and a LCDR3 comprising the sequence of SEQ ID NO: 37.

In certain embodiments, the present disclosure provides anti-SIRPα antibodies and antigen-binding fragments thereof comprising a HCDR1 comprising the sequence of SEQ ID NO: 43, a HCDR2 comprising the sequence of SEQ ID NO: 44, a HCDR3 comprising the sequence of SEQ ID NO: 45, a LCDR1 comprising the sequence of SEQ ID NO: 46, a LCDR2 comprising the sequence of SEQ ID NO: 47, and a LCDR3 comprising the sequence of SEQ ID NO: 48.

CDRs are known to be responsible for antigen binding. However, it has been found that not all of the 6 CDRs are indispensable or unchangeable. In other words, it is possible to replace or change or modify one or more CDRs in anti-SIRPα antibodies 005, 015, 025, 042, 059, 071 and 073 or anti-SIRPα chimeric antibodies 005c, 015c, 025c, 042c, 059c, 071c and 073c, yet substantially retain the specific binding specificity and/or affinity to SIRPα.

In certain embodiments, the antibodies and antigen-binding fragments thereof provided herein comprise suitable framework region (FR) sequences, as long as the antibodies and antigen-binding fragments thereof can specifically bind to SIRPα. The CDR sequences provided in Table 1 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

Table 3 below shows the CDR amino acid sequences of 5 humanized antibodies for antibody 025, which are designated as hu025.021, hu025.023, hu025.033, hu025.059 and hu025.060. The CDR boundaries were defined or identified by the convention of Kabat. Table 3 below shows the amino acid sequences for the six CDRs of 5 humanized antibodies hu025.021, hu025.023, hu025.033, hu025.059 and hu025.060. Table 4 below shows the heavy chain and light chain variable region amino acid sequences of 5 humanized antibodies hu025.021, hu025.023, hu025.033, hu025.059 and hu025.060. Table 5 below shows the FR amino acid sequences of 5 humanized antibodies hu025.021, hu025.023, hu025.033, hu025.059 and hu025.060.

TABLE 3
CDR amino acid sequences of 5 humanized antibodies

| Antibody | | CDR1 | CDR2 | CDR3 |
|---|---|---|---|---|
| hu025.021/ | HCDR | SEQ ID NO: 13 | SEQ ID NO: 17 | SEQ ID NO: 15 |
| hu025.023/ | | DYYMH | RIDPEDAETKYAPKFQ | GLAY |
| hu025.033/ | | | G | |
| hu025.059/ | LCDR | SEQ ID NO: 10 | SEQ ID NO: 11 | SEQ ID NO: 16 |
| hu025.060 | | SASSSVSSSYLY | STSNLAS | HQWSSYPYT |

TABLE 4
Variable region amino acid sequences of 5 humanized antibodies

| Antibody | VH | VL |
|---|---|---|
| hu025.021 | SEQ ID NO: 63 | SEQ ID NO: 64 |
| | EVQLVQSGAEVKKPGATVKISCKVSG | EIVLTQSPATLSLSPGERATLSCSASS |
| | FNIKDYYMHWVQQAPGKGLEWIGR | SVSSSYLYWYQQKPGQAPKLWIYST |
| | IDPEDAETKYAPKFQGRVTITADTSTN | SNLASGIPARFSGSGSGTDYTLTISSL |
| | TAYMELSSLRSEDTAVYYCDRGLAYW | EPEDFAVYYCHQWSSYPYTFGQGT |
| | GQGTLVTVSS | KLEIK |
| hu025.023 | SEQ ID NO: 63 | SEQ ID NO: 66 |
| | EVQLVQSGAEVKKPGATVKISCKVSG | EIVLTQSPATLSLSPGERATLSCSASS |
| | FNIKDYYMHWVQQAPGKGLEWIGR | SVSSSYLYWYQQKPGQAPKLWIYST |
| | IDPEDAETKYAPKFQGRVTITADTSTN | SNLASGIPARFSGSGSGTDFTLTISSL |
| | TAYMELSSLRSEDTAVYYCDRGLAYW | EPEDFAVYYCHQWSSYPYTFGQGT |
| | GQGTLVTVSS | KLEIK |
| hu025.033 | SEQ ID NO: 65 | SEQ ID NO: 64 |
| | EVQLVQSGAEVKKPGATVKISCKVSG | EIVLTQSPATLSLSPGERATLSCSASS |
| | FNIKDYYMHWVQQAPGKGLEWIGR | SVSSSYLYWYQQKPGQAPKLWIYST |
| | IDPEDAETKYAPKFQGRVTITADTSTD | SNLASGIPARFSGSGSGTDYTLTISSL |
| | TAYMELSSLRSEDTAVYYCDRGLAYW | EPEDFAVYYCHQWSSYPYTFGQGT |
| | GQGTLVTVSS | KLEIK |
| hu025.059 | SEQ ID NO: 67 | SEQ ID NO: 64 |
| | EVQLVQSGAEVKKPGATVKISCKASG | EIVLTQSPATLSLSPGERATLSCSASS |
| | FNIKDYYMHWVQQAPGKGLEWIGR | SVSSSYLYWYQQKPGQAPKLWIYST |
| | IDPEDAETKYAPKFQGRVTITADTSTN | SNLASGIPARFSGSGSGTDYTLTISSL |
| | TAYMELSSLRSEDTAVYYCDRGLAYW | EPEDFAVYYCHQWSSYPYTFGQGT |
| | GQGTLVTVSS | KLEIK |
| hu025.060 | SEQ ID NO: 67 | SEQ ID NO: 66 |
| | EVQLVQSGAEVKKPGATVKISCKASG | EIVLTQSPATLSLSPGERATLSCSASS |
| | FNIKDYYMHWVQQAPGKGLEWIGR | SVSSSYLYWYQQKPGQAPKLWIYST |
| | IDPEDAETKYAPKFQGRVTITADTSTN | SNLASGIPARFSGSGSGTDFTLTISSL |
| | TAYMELSSLRSEDTAVYYCDRGLAYW | EPEDFAVYYCHQWSSYPYTFGQGT |
| | GQGTLVTVSS | KLEIK |

TABLE 5
FR amino acid sequences of 5 humanized antibodies

| Anti- | | | | | |
|---|---|---|---|---|---|
| body | | FR1 | FR2 | FR3 | FR4 |
| hu025.021 | HFR | SEQ ID NO: 73 | SEQ ID NO: 74 | SEQ ID NO: 75 | SEQ ID NO: |
| | | EVQLVQSGAEVK | WVQQAPGKGLE | RVTITADTSTNTA | 76 |
| | | KPGATVKISCKVS | WIG | YMELSSLRSEDTA | WGQGTLVTV |
| | | GFNIK | | VYYCDR | SS |
| | LFR | SEQ ID NO: 77 | SEQ ID NO: 78 | SEQ ID NO: 79 | SEQ ID NO: |
| | | EIVLTQSPATLSLS | WYQQKPGQAPK | GIPARFSGSGSGT | 80 |
| | | PGERATLSC | LWIY | DYTLTISSLEPEDF | FGQGTKLEIK |
| | | | | AVYYC | |
| hu025.023 | HFR | SEQ ID NO: 73 | SEQ ID NO: 74 | SEQ ID NO: 75 | SEQ ID NO: |
| | | EVQLVQSGAEVK | WVQQAPGKGLE | RVTITADTSTNTA | 76 |
| | | KPGATVKISCKVS | WIG | YMELSSLRSEDTA | WGQGTLVTV |
| | | GFNIK | | VYYCDR | SS |
| | LFR | SEQ ID NO: 77 | SEQ ID NO: 78 | SEQ ID NO: 81 | SEQ ID NO: |
| | | EIVLTQSPATLSLS | WYQQKPGQAPK | GIPARFSGSGSGT | 80 |
| | | PGERATLSC | LWIY | DFTLTISSLEPEDF | FGQGTKLEIK |
| | | | | AVYYC | |
| hu025.033 | HFR | SEQ ID NO: 73 | SEQ ID NO: 74 | SEQ ID NO: 82 | SEQ ID NO: |
| | | EVQLVQSGAEVK | WVQQAPGKGLE | RVTITADTSTDTA | 76 |
| | | KPGATVKISCKVS | WIG | YMELSSLRSEDTA | WGQGTLVTV |
| | | GFNIK | | VYYCDR | SS |
| | LFR | SEQ ID NO: 77 | SEQ ID NO: 78 | SEQ ID NO: 79 | SEQ ID NO: |
| | | EIVLTQSPATLSLS | WYQQKPGQAPK | GIPARFSGSGSGT | 80 |
| | | PGERATLSC | LWIY | DYTLTISSLEPEDF | FGQGTKLEIK |
| | | | | AVYYC | |
| hu025.059 | HFR | SEQ ID NO: 83 | SEQ ID NO: 74 | SEQ ID NO: 75 | SEQ ID NO: |
| | | EVQLVQSGAEVK | WVQQAPGKGLE | RVTITADTSTNTA | 76 |
| | | KPGATVKISCKAS | WIG | YMELSSLRSEDTA | WGQGTLVTV |
| | | GFNIK | | VYYCDR | SS |
| | LFR | SEQ ID NO: 77 | SEQ ID NO: 78 | SEQ ID NO: 79 | SEQ ID NO: |
| | | EIVLTQSPATLSLS | WYQQKPGQAPK | GIPARFSGSGSGT | 80 |
| | | PGERATLSC | LWIY | DYTLTISSLEPEDF | FGQGTKLEIK |
| | | | | AVYYC | |
| hu025.060 | HFR | SEQ ID NO: 83 | SEQ ID NO: 74 | SEQ ID NO: 75 | SEQ ID NO: |
| | | EVQLVQSGAEVK | WVQQAPGKGLE | RVTITADTSTNTA | 76 |
| | | KPGATVKISCKAS | WIG | YMELSSLRSEDTA | WGQGTLVTV |
| | | GFNIK | | VYYCDR | SS |
| | LFR | SEQ ID NO: 77 | SEQ ID NO: 78 | SEQ ID NO: 81 | SEQ ID NO: |
| | | EIVLTQSPATLSLS | WYQQKPGQAPK | GIPARFSGSGSGT | 80 |
| | | PGERATLSC | LWIY | DFTLTISSLEPEDF | FGQGTKLEIK |
| | | | | AVYYC | |
| | HFR | SEQ ID NO: 84 | | SEQ ID NO: 85 | |
| | | EVQLVQSGAEVK | | RVTITADTSTX₂₁T | |
| | | KPGATVKISCKX₂₀ | | AYMELSSLRSEDT | |
| | | SGFNIK | | AVYYCDR | |
| | LFR | | | SEQ ID NO: 86 | |
| | | | | GIPARFSGSGSGT | |
| | | | | DX₂₂TLTISSLEPE | |
| | | | | DFAVYYC | |

X₂₀ is A or V; X₂₁ is N or D; X₂₂ is Y or F.

In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1-4, and/or light chain LFR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the present disclosure also provides humanized anti-SIRPα antibodies and antigen-binding fragments thereof comprising a heavy chain HFR1 comprising the sequence of EVQLVQSGAEVKKPGATVKISCKX₂₀SGFNIK (SEQ ID NO: 84) or a homologous sequence of at least 80% sequence identity thereof, a heavy chain HFR2 comprising the sequence of WVQQAPGKGLEWIG (SEQ ID NO: 74) or a homologous sequence of at least 80% sequence identity thereof, a heavy chain HFR3 comprising the sequence of RVTITADTSTX₂₁TAYMELSSLRSEDTAVYYCDR (SEQ ID NO: 85) or a homologous sequence of at least 80% sequence identity thereof, and a heavy chain HFR4 comprising the sequence of WGQGTLVTVSS (SEQ ID NO: 76) or a homologous sequence of at least 80% sequence identity thereof, wherein X₂₀ is A or V; X₂₁ is N or D.

In certain embodiments, the present disclosure also provides humanized anti-SIRPα antibodies and antigen-binding fragments thereof comprising a light chain LFR1 comprising the sequence of EIVLTQSPATLSLSPGERATLSC (SEQ ID NO: 77) or a homologous sequence of at least 80% sequence identity thereof, a light chain LFR2 comprising the sequence of WYQQKPGQAPKLWIY (SEQ ID NO: 78) or a homologous sequence of at least 80% sequence identity thereof, a light chain LFR3 comprising the sequence of GIPARFSGSGSGTDX₂₂TLTISSLEPEDFAVYYC (SEQ ID NO: 86) or a homologous sequence of at least 80% sequence identity thereof, and a light chain LFR4 comprising the sequence of FGQGTKLEIK (SEQ ID NO: 80) or a homologous sequence of at least 80% sequence identity thereof, wherein X₂₂ is Y or F.

In certain embodiments, the present disclosure also provides humanized anti-SIRPα antibodies and antigen-binding fragments thereof comprising a heavy chain HFR1 comprising a sequence selected from the group consisting of SEQ ID NOs: 73, and 83, a heavy chain HFR2 comprising the sequence of SEQ ID NO: 74, a heavy chain HFR3 comprising a sequence selected from the group consisting of SEQ ID NOs: 75, and 82, and a heavy chain HFR4 comprising a sequence of SEQ ID NO: 76; and/or a light chain LFR1 comprising a sequence from the group consisting of SEQ ID NO: 77, a light chain LFR2 comprising a sequence selected from the group consisting of SEQ ID NOs: 78, a light chain LFR3 comprising a sequence selected from the group consisting of SEQ ID NOs: 79 and 81, and a light chain LFR4 comprising a sequence selected from the group consisting of SEQ ID NO: 80.

In certain embodiments, the present disclosure also provides humanized anti-SIRPα antibodies and antigen-binding fragments thereof comprising HFR1, HFR2, HFR3, and/or HFR4 sequences contained in a heavy chain variable region selected from a group consisting of: hu025.021-VH/hu025.023-VH (SEQ ID NO: 63), hu025.033-VH (SEQ ID NO: 65) and hu025.059-VH/hu025.060-VH (SEQ ID NO: 67).

In certain embodiments, the present disclosure also provides humanized anti-SIRPα antibodies and antigen-binding fragments thereof comprising LFR1, LFR2, LFR3, and/or LFR4 sequences contained in a light chain variable region selected from a group consisting of: hu025.021-VL/hu025.033-VL/hu025.059-VL (SEQ ID NO: 64), and hu025.023-VL/hu025.060-VL (SEQ ID NO: 66).

In certain embodiments, the humanized anti-SIRPα antibodies and antigen-binding fragments thereof provided herein comprise a heavy chain variable domain sequence selected from the group consisting of SEQ ID NO: 63, SEQ ID NO: 65, and SEQ ID NO: 67; and/or a light chain variable domain sequence selected from the group consisting of SEQ ID NO: 64 and SEQ ID NO: 66.

The present disclosure also provides exemplary humanized antibodies of 025, including:
1) antibody “hu025.021” comprising the heavy chain variable region of SEQ ID NO: 63 and the light chain variable region of SEQ ID NO: 64;
2) antibody “hu025.023” comprising the heavy chain variable region of SEQ ID NO: 63, and the light chain variable region of SEQ ID NO: 66;
3) antibody “hu025.033” comprising the heavy chain variable region of SEQ ID NO: 65, and the light chain variable region of SEQ ID NO: 64;
4) antibody “hu025.059” comprising the heavy chain variable region of SEQ ID NO: 67, and the light chain variable region of SEQ ID NO: 64; and
5) antibody “hu025.060” comprising the heavy chain variable region of SEQ ID NO: 67, and the light chain variable region of SEQ ID NO: 66.

These exemplary humanized anti-SIRPα antibodies retained the specific binding capacity or affinity to SIRPα, and are at least comparable to, or even better than, the parent mouse antibody 025 in that aspect. Detailed information is provided in Example 5.2.

In some embodiments, the anti-SIRPα antibodies and antigen-binding fragments provided herein comprise all or a portion of the heavy chain variable domain and/or all or a portion of the light chain variable domain. In one embodiment, the anti-SIRPα antibody or an antigen-binding fragment thereof provided herein is a single domain antibody which consists of all or a portion of the heavy chain variable domain provided herein. More information of such a single domain antibody is available in the art (see, e.g. U.S. Pat. No. 6,248,516).

In certain embodiments, the anti-SIRPα antibodies or the antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-SIRPα antibodies or the antigen-binding fragments thereof provided herein comprises heavy chain constant regions of human IgG1, IgG2, IgG3, or IgG4. In certain embodiments, the light chain constant region comprises Cκ or Cλ. The constant region of the anti-SIRPα antibodies or the antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-SIRPα antibodies and antigen-binding fragments thereof provided herein comprises an Fc region of IgG1 or IgG3 isotype, which could induce ADCC or CDC; or alternatively, a constant region of IgG4 or IgG2 isotype, which has reduced or depleted effector function. In certain embodiments, the anti-SIRPα antibodies or antigen-binding fragments thereof provided herein comprise a wild type human IgG4 Fc region or other wild type human IgG4 alleles.

In certain embodiments, the anti-SIRPα antibodies or antigen-binding fragments thereof provided herein has reduced effector functions. In certain embodiments, the anti-SIRPα antibodies or antigen-binding fragments thereof provided herein comprise an Fc region of IgG1 isotype and comprise one or more amino acid substitution(s) to reduce or eliminate effector functions. Exemplary of such substitution(s) in IgG1 can be at a position selected from the group consisting of: 234, 235, 237, and 238, 268, 297, 309, 330, and 331. In certain embodiments, the anti-SIRPα antibodies or antigen-binding fragments thereof provided herein is of IgG1 isotype and comprise one or more amino acid substitution(s) selected from the group consisting of: N297A, N297Q, N297G, L235E, L234A, L235A, L234F, L235E, P331S, and any combination thereof.

In certain embodiments, the anti-SIRPα antibodies or antigen-binding fragments thereof provided herein is of IgG2 isotype, and comprises one or more amino acid substitution(s) to reduce or eliminate effector functions, selected from the group consisting of: H268Q, V309L, A330S, P331S, V234A, G237A, P238S, H268A, and any combination thereof (e.g. H268Q/V309L/A330S/P331S, V234A/G237A/P238S/H268A/V309L/A330S/P331S).

In certain embodiments, the anti-SIRPα antibodies or antigen-binding fragments thereof provided herein is of IgG4 isotype, and comprises one or more amino acid substitution(s) to reduce or eliminate effector functions. Exemplary of such substitution(s) in IgG4 can be at a position selected from the group consisting of: 228, 234, 235,237, 238, 265, 297, 322, 329 and 331. Examples of such substitution(s) include without limitation, S228P, L235E, L234A, L235A, N297A, N297Q, N297G, P329G, K322Q, P331S, D265A, G237A, P238S, and any combination thereof.

In certain embodiments, the anti-SIRPα antibodies and antigen-binding fragments provided herein is of IgG4 isotype and comprises one or more amino acid substitution(s) at one or more points of 228 and 235. In certain embodiments, the anti-SIRPα antibodies and antigen-binding fragments provided herein is of IgG4 isotype and comprises S228P mutation in the Fc region. In certain embodiments, the anti-SIRPα antibodies and antigen-binding fragments provided herein is of IgG4 isotype and comprises L235E mutation in the Fc region.

In certain embodiments, the anti-SIRPα antibodies or antigen-binding fragments thereof provided herein is of IgG2/IgG4 cross isotype. Examples of IgG2/IgG4 cross isotype is described in Rother R P et al., Nat Biotechnol 25:1256-1264 (2007).

In certain embodiments, the antibodies or the antigen-binding fragments thereof provided herein have a specific binding affinity to human SIRPα which is sufficient to provide for diagnostic and/or therapeutic use.

The antibodies or antigen-binding fragments thereof provided herein can be a monoclonal antibody, a polyclonal antibody, a humanized antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-SIRPα antibody or antigen-binding fragment thereof, which competes for binding to SIRPα with the antibody or antigen-binding fragment thereof provided herein.

In certain embodiments, the present disclosure provides an anti-SIRPα antibody or antigen-binding fragment thereof, which competes for binding to human SIRPα with an antibody comprising a heavy chain variable region comprising the sequence of SEQ ID NO: 53, and a light chain variable region comprising the sequence of SEQ ID NO: 54.

In certain embodiments, the present disclosure provides an anti-SIRPα antibody or antigen-binding fragment thereof, which competes for binding to human SIRPα with an antibody comprising a heavy chain variable region comprising the sequence of SEQ ID NO: 55, and a light chain variable region comprising the sequence of SEQ ID NO: 56.

In certain embodiments, the present disclosure provides an anti-SIRPα antibody or antigen-binding fragment thereof, which competes for binding to human SIRPα with an antibody comprising a heavy chain variable region comprising the sequence of SEQ ID NO: 61, and a light chain variable region comprising the sequence of SEQ ID NO: 62.

In certain embodiments, the present disclosure provides an anti-SIRPα antibody or antigen-binding fragment thereof, which binds to an epitope different from that bound by HEFLB or hu1H9G4.

“HEFLB” as used herein refers to an antibody or antigen binding fragment thereof comprising a heavy chain variable region having an amino acid sequence of SEQ ID NO: 68, and a light chain variable region having an amino acid sequence of SEQ ID NO: 69.

“hu1H9G4” as used herein refers to an antibody or antigen binding fragment thereof comprising a heavy chain variable region having an amino acid sequence of SEQ ID NO: 70, and a light chain variable region having an amino acid sequence of SEQ ID NO: 71.

Table 6 shows the VH and VL amino acid sequences of HEFLB and hu1H9G4.

TABLE 6
Variable region amino acid sequences of HEFLB and hu1H9G4

| Antibody | VH | VL |
|---|---|---|
| HEFLB | SEQ ID NO: 68 | SEQ ID NO: 69 |
| | EVQLVQSGAEVKKPGESLRISC | DVVMTQSPLSLPVTLGQPASI |
| | KASGYSFTSYWVHWVRQMPG | SCRSSQSLVHSYGNTYLYWF |
| | KGLEWMGNIDPSDSDTHYSPSF | QQRPGQSPRLLIYRVSNRFSG |
| | QGHVTLSVDKSISTAYLQLSSL | VPDRFSGSGSGTDFTLKISRV |
| | KASDTAMYYCVRGGTGTLAYF | EAEDVGVYYCFQGTHVPYT |
| | AYWGQGTLVTVSS | FGGGTKVEIK |
| hu1H9G4 | SEQ ID NO: 70 | SEQ ID NO: 71 |
| | QVQLVQSGAEVKKPGASVKVS | DIQMTQSPSSLSASVGDRVTI |
| | CKASGYTFTSYWITWVKQAPG | TCRASENIYSYLAWYQQKPG |
| | QGLEWIGDIYPGSGSTNHIEKF | KAPKLLIYTAKTLAEGVPSR |
| | KSKATLTVDTSISTAYMELSRLR | FSGSGSGTDFTLTISSLQPEDF |
| | SDDTAVYYCATGYGSSYGYFD | ATYYCQHQYGPPFTFGQGTK |
| | YWGQGTLVTVSS | LEIK |

### Antibody Variants

The antibodies and antigen-binding fragments thereof provided herein also encompass various variants of the antibody sequences provided herein.

In certain embodiments, the antibody variants comprise one or more modifications or substitutions in one or more of the CDR sequences as provided in Tables 1 and 3 above, one or more of the non-CDR sequences of the heavy chain variable region or light chain variable region provided in Tables 2 and 4 above, and/or the constant region (e.g. Fc region). Such variants retain binding specificity to SIRPα of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g. charged residues such as Arg, Asp, His, Lys, and Glu) can be identified and replaced by a neutral or negatively charged amino acid (e.g. alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variants

Affinity variants of antibodies may contain modifications or substitutions in one or more CDR sequences as provided in Tables 1 and 3 above, one or more FR sequences as provided in Table 5 above, or the heavy or light chain variable region sequences provided in Tables 2 and 4 above. FR sequences can be readily identified by a person skilled in the art based on the CDR sequences in Tables 1 and 3 above and variable region sequences in Tables 2 and 4 above, as it is well-known in the art that a CDR region is flanked by two FR regions in the variable region. The affinity variants retain specific binding affinity to SIRPα of the parent antibody, or even have improved SIRPα specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Tables 1 and 3 above, and variable region sequences provided in Tables 2 and 4 above, one or more amino acid residues may be substituted yet the resulting antibody or antigen-binding fragment still retain the binding affinity or binding capacity to SIRPα, or even have an improved binding affinity or capacity. Various methods known in the art can be used to achieve this purpose. For example, a library of antibody variants (such as Fab or scFv variants) can be generated and expressed with phage display technology, and then screened for the binding affinity to human SIRPα. For another example, computer software can be used to virtually simulate the binding of the antibodies to human SIRPα, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-SIRPα antibodies or antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Tables 1 and 3 above yet retaining the specific binding affinity to SIRPα at a level similar to or even higher than its parent antibody.

In certain embodiments, the anti-SIRPα antibodies or antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) listed in Tables 2 and 4 above yet retaining the specific binding affinity to SIRPα at a level similar to or even higher than its parent antibody. In some embodiments, a total of 1 to 10 amino acids have been substituted, inserted, or deleted in a variable region sequence listed in Tables 2 and 4 above. In some embodiments, the substitutions, insertions, or deletions occur in regions outside the CDRs (e.g. in the FRs).

### Glycosylation Variants

The anti-SIRPα antibodies or antigen-binding fragments thereof provided herein also encompass glycosylation variants, which can be obtained to either increase or decrease the extent of glycosylation of the antibodies or antigen binding fragments thereof.

The antibodies or antigen binding fragments thereof may comprise one or more modifications that introduce or remove a glycosylation site. A glycosylation site is an amino acid residue with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

In certain embodiments, the anti-SIRPα antibodies and antigen-binding fragments provided herein comprise a mutation at N297 (e.g. N297A, N297Q, or N297G) to remove the glycosylation site.

### Cysteine-Engineered Variants

The anti-SIRPα antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-SIRPα antibodies or antigen-binding fragments thereof provided herein also encompass Fc variants, which comprise one or more amino acid residue modifications or substitutions at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276 (9): 6591-604; Idusogie E E. et al., J Immunol. 2000.164 (8): 4178-84; Steurer W. et al., J Immunol. 1995, 155 (3): 1165-74; Idusogie E E. et al., J Immunol. 2001, 166 (4): 2571-5; Lazar G A. et al., PNAS, 2006, 103 (11): 4005-4010; Ryan M C. et al., Mol. Cancer Ther., 2007, 6:3009-3018; Richards J O, et al., Mol Cancer Ther. 2008, 7 (8): 2517-27; Shields R. L. et al., J. Biol. Chem, 2002, 277:26733-26740; Shinkawa T. et al., J. Biol. Chem, 2003, 278:3466-3473.

CDC activity of the antibodies or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821; and WO94/29351 concerning other examples of Fc region variants). One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-SIRPα antibodies or antigen-binding fragments thereof provided herein can be of IgG1, IgG2, IgG3, or IgG4 isotype and has reduced effector functions, as disclosed herein.

In certain embodiments, the anti-SIRPα antibodies or antigen-binding fragments thereof comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6 (1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70:3269-3277 (2010); and Hinton, P. et al., J. Immunology, 176:346-356 (2006).

In certain embodiments, anti-SIRPα antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g. as described in U.S. Pat. No. 5,731,168.

### Antigen-Binding Fragments

Provided herein are also anti-SIRPα antigen-binding fragments. Various types of antigen-binding fragments are known in the art and can be developed based on the anti-SIRPα antibodies provided herein, including for example, the exemplary antibodies whose CDRs are shown in Tables 1 and 3 above, and variable sequences are shown in Tables 2 and 4 above, and their different variants (such as affinity variants, glycosylation variants, Fc variants, cysteine-engineered variants and so on).

In certain embodiments, an anti-SIRPα antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-SIRPα antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the anti-SIRPα antibodies or antigen-binding fragments thereof is bispecific.

In certain embodiments, the anti-SIRPα antibodies or antigen-binding fragments thereof is capable of specifically binding to a second antigen other than SIRPα. In certain embodiments, the second antigen is a tumor antigen, tumor surface antigen, or an infectious agent surface antigen. In certain embodiments, the second antigen is selected from the group consisting of CD19, CD20, CD22, CD24, CD25, CD30, CD33, CD38, CD44, CD52, CD56, CD70, CD96, CD97, CD99, CD123, CD279 (PD-1), CD274 (PD-L1), GPC-3, B7-H3, B7-H4, TROP2, CLDN18.2, EGFR, HER2, CD117, C-Met, PTHR2, and HAVCR2 (TIM3).

In certain embodiments, the bispecific antibodies or antigen-binding fragments thereof provided herein are capable of specifically binding to a second epitope on SIRPα.

### Conjugates

In some embodiments, the anti-SIRPα antibodies or antigen-binding fragments thereof further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding, affinity binding, intercalation, coordinate binding, complexation, association, blending, or addition, among other methods. In some embodiments, the antibodies or antigen-binding fragments thereof can be linked to one or more conjugates via a linker.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g. a polymer such as PEG which extends half-life), a chemotherapeutic agent, a toxin, a radioactive isotope, a lanthanide, a detectable label (e.g. a luminescent label, a fluorescent label, an enzyme-substrate label), a DNA-alkylator, a topoisomerase inhibitor, a tubulin-binder, a purification moiety or other anticancer drugs.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), anti-mitotic agents (e.g. vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moieties, digoxigenin, biotin/avidin, DNA molecules or gold for detection.

In certain embodiments, the conjugate moiety can be a clearance-modifying agent which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibodies or antigen-binding fragments thereof provided herein is used as a base for a conjugate.

### Polynucleotides and Recombinant Methods

The present disclosure provides isolated polynucleotides that encode the anti-SIRPα antibodies or antigen-binding fragments thereof provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The isolated polynucleotide that encodes the anti-SIRPα antibodies or antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors comprising the isolated polynucleotide provided herein. In certain embodiments, the polynucleotide provided herein encodes the antibodies or antigen-binding fragments thereof, at least one promoter (e.g. SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-SIRPα antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for anti-SIRPα antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-SIRPα antibodies or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g. from about 0-0.25M salt).

### Pharmaceutical Composition

The present disclosure further provides pharmaceutical compositions comprising the anti-SIRPα antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-SIRPα antibody or antigen-binding fragment thereof or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the antibody or an antigen-binding fragment thereof provided herein.

In certain embodiments, the present disclosure provides a kit comprising the antibody or an antigen-binding fragment thereof provided herein, and a target antibody that binds to a target antigen expressed on the target cell. In certain embodiments, the target cell can be a tumor cell, an inflammatory cell, and/or a chronically infected cell that express CD47.

In certain embodiments, the target antigen is a tumor antigen, tumor surface antigen, or an infectious agent surface antigen.

In certain embodiments, the kits further comprise an additional therapeutic agent. The additional therapeutic agent can be an anti-cancer therapeutic agent, anti-inflammatory agent or an anti-infection agent.

In certain embodiments, the additional therapeutic agent is selected from the group consisting of a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

In another aspect, the present disclosure provides a method of inducing phagocytosis of a target cell in vitro, comprising contacting the target cell with a SIRPα positive phagocytic cell sample in the presence of the antibody or an antigen-binding fragment thereof provided herein, thereby inducing the phagocytosis of the target cell by the SIRPα positive phagocytic cell.

In another aspect, the present disclosure provides a method of inducing phagocytosis of a target cell in vitro, comprising contacting the target cell with a SIRPα positive phagocytic cell sample in the presence of the antibody or an antigen-binding fragment thereof provided herein and a target antibody that specifically binds to a target antigen on the target cell, thereby inducing the phagocytosis of the target cell by the SIRPα positive phagocytic cell.

In some embodiments, the target cell is a CD47 expressing cell.

In one aspect, the present disclosure provides a method of inducing phagocytosis of a target cell in a subject, comprising administering to the subject the antibody or an antigen-binding fragment thereof provided herein and/or the pharmaceutical composition provided herein in a dose effective to induce phagocytosis of the target cell.

In one aspect, the present disclosure provides a method of inducing phagocytosis of a target cell in a subject, comprising administering to the subject the antibody or an antigen-binding fragment thereof provided herein and/or the pharmaceutical composition provided herein in combination with a target antibody that specifically binds to a target antigen on the target cell, in a dose effective to induce phagocytosis of the target cell.

In one aspect, the present disclosure provides a method of increasing antibody-dependent cellular phagocytosis (ADCP) effect of a target antibody on a target cell in a subject, comprising: administering to the subject a therapeutically effective amount of the antibody or an antigen-binding fragment thereof provided herein and/or the pharmaceutical composition provided herein, in combination with the target antibody having Fc region, thereby increasing ADCP of the target antibody on the target cell, wherein the target antibody binds to a target antigen expressed on the target cell. In certain embodiments, the target antibody binds to a target antigen expressed on the target cell, and the ADCP effects of the target antibody on the target cell is increased. The target cell can be a tumor cell, an inflammatory cell, and/or a chronically infected cell that express CD47.

In one aspect, the present disclosure provides a method of potentiating a target antibody (e.g., anti-CD20 antibody, anti-PD-L1 antibody and anti-Claudin18.2 antibody) in treating a disease, disorder or condition in a subject, comprising: administering to the subject a therapeutically effective amount of the antibody or an antigen-binding fragment thereof provided herein and/or the pharmaceutical composition provided herein, in combination with the target antibody (e.g., anti-CD20 antibody, anti-PD-L1 antibody and anti-Claudin18.2 antibody), thereby potentiating the target antibody in treating the disease, disorder or condition in the subject. As used herein, the term “potentiate” or “potentiating” refers increasing therapeutic efficacy.

In certain embodiments, the target antibody has an Fc region. In certain embodiments, the disease, disorder or condition is immune related disease or disorder, tumors and cancers, autoimmune diseases, or infectious disease. In certain embodiments, the immune related disease or disorder is selected from the group consisting of systemic lupus erythematosus, acute respiratory distress syndrome (ARDS), vasculitis, myasthenia gravis, idiopathic pulmonary fibrosis, Crohn's Disease, asthma, rheumatoid arthritis, graft versus host disease, a spondyloarthropathy (e.g., ankylosing spondylitis, psoriatic arthritis, isolated acute enteropathic arthritis associated with inflammatory bowel disease, reactive arthritis, Behcet's syndrome, undifferentiated spondyloarthropathy, anterior uveitis, and juvenile idiopathic arthritis.), multiple sclerosis, endometriosis, glomerulonephritis, sepsis, diabetes, acute coronary syndrome, ischemic reperfusion, psoriasis, progressive systemic sclerosis, atherosclerosis, Sjogren's syndrome, scleroderma, or inflammatory autoimmune myositis.

In certain embodiments, the condition or a disorder treatable by the methods provided herein include tumors and cancers. Examples of cancers and tumors include, non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, brain stem glioma, anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma, or the metastases thereof.

In another aspect, the present disclosure also provides methods of treating a disease, disorder or condition that can be benefited from induced phagocytosis of a target cell in a subject, comprising administering to the subject a therapeutically effective amount of the antibody or antigen-binding fragment thereof provided herein, and/or the pharmaceutical composition provided herein

In another aspect, the present disclosure also provides methods of treating a disease, disorder or condition that can be benefited from induced phagocytosis of a target cell in a subject, comprising administering to the subject a therapeutically effective amount of the antibody or antigen-binding fragment thereof provided herein, and/or the pharmaceutical composition provided herein in combination with a target antibody that specifically binds to a target antigen on the target cell.

In another aspect, the present disclosure also provides methods of treating a SIRPα related disease, disorder or condition in a subject, comprising administering to the subject a therapeutically effective amount of the antibody or antigen-binding fragment thereof provided herein, and/or the pharmaceutical composition provided herein.

In another aspect, the present disclosure also provides methods of treating a SIRPα related disease, disorder or condition in a subject, comprising administering to the subject a therapeutically effective amount of the antibody or antigen-binding fragment thereof provided herein, and/or the pharmaceutical composition provided herein, in combination with a target antibody that specifically binds to a target antigen on a target cell associated with the SIRPα related disease.

In some embodiments, the target cell is a CD47 expressing cell. In some embodiments, the target cells comprise cancer cells, inflammatory cells, and/or chronically infected cells.

In certain embodiments, when the antibody or an antigen-binding fragment thereof provided herein is used in combination with a target antibody, the antibody or an antigen-binding fragment thereof provided herein can induce selective phagocytosis of the target cell over a non-target cell (e.g. those which do not express the target antigen).

In some embodiments, the target cell expresses a target antigen. In some embodiments, the target antigen is a tumor antigen, tumor surface antigen, an inflammatory antigen, or an antigen of an infectious microorganism. In some embodiments, the target antigen can be tumor antigen (e.g., tumor associated antigens (TAA), tumor specific antigen (TSA), such as neoantigen), or antigens presented on infected cells (e.g., Hepatitis B surface antigen (HBsAg)).

In some embodiments, the subject is human. In some embodiments, the subject is homozygous for SIRPα v1. In some embodiments, the subject is homozygous for SIRPα v2. In some embodiments, the subject is heterozygous SIRPα v1/v2.

In some embodiments, the subject has a disease, disorder or condition selected from the group consisting of cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, and arthritis.

In some embodiments, the cancer is a CD47-positive cancer. In some embodiments, the subject to be treated has been identified as having a CD47-positive cancer. “CD47-positive” cancer as used herein refers to a cancer characterized in expressing CD47 protein in a cancer cell, or expressing CD47 in a cancer cell at a level significantly higher than that would have been expected of a normal cell. The presence and/or amount of CD47 in an interested biological sample can be indicative of whether the subject from whom the biological sample is derived could likely respond to an anti-SIRPα antibody. Various methods can be used to determine the presence and/or amount of CD47 in a test biological sample from the subject. For example, the test biological sample can be exposed to anti-CD47 antibody or antigen-binding fragment thereof, which binds to and detects the expressed CD47 protein. Alternatively, CD47 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. In certain embodiments, presence or up-regulated level of the CD47 in the test biological sample indicates likelihood of responsiveness. The term “up-regulated” as used herein, refers to an overall increase of no less than 10%, 15%, 20%, 25%, 30%, 35%, 40%, 45%, 50%, 55%, 60%, 65%, 70%, 75%, 80% or greater, in the expression level of CD47 in the test sample, as compared to the CD47 expression level in a reference sample as detected using the same method. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor). The reference level can be the level of CD47 expression found in normal cells of the same tissue type, optionally normalized to expression level of another gene (e.g. a house keeping gene). Alternatively, the reference level can be the level of CD47 expression found in healthy subjects. The reference sample can be a control sample obtained from a healthy or non-diseased individual, or a healthy or non-diseased sample obtained from the same individual from whom the test sample is obtained. In some embodiments, a reference is tested and/or determined substantially simultaneously with the testing or determination of interest. In some embodiments, a reference is a historical reference, optionally embodied in a tangible medium. Typically, as would be understood by the skilled person in the art, a reference is determined or characterized under comparable conditions or circumstances to those under assessment.

In certain of these embodiments, an antibody or antigen-binding fragment thereof provided herein that is administered in combination with the target antibody or one or more additional therapeutic agents may be administered simultaneously with the target antibody or the one or more additional therapeutic agents, and in certain of these embodiments the antibody or antigen-binding fragment thereof and the target antibody or the additional therapeutic agent(s) may be administered as part of the same pharmaceutical composition. However, an antibody or antigen-binding fragment thereof administered “in combination” with the target antibody or an additional therapeutic agent does not have to be administered simultaneously with or in the same composition as the agent. An antibody or antigen-binding fragment thereof administered prior to or after the target antibody or another agent is considered to be administered “in combination” with that agent as the phrase is used herein, even if the antibody or antigen-binding fragment and the target antibody or the second agent are administered via different routes. Where possible, the target antibody or additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In another aspect, methods are provided to treat a disease, disorder or condition in a subject that would benefit from modulation of SIRPα activity, comprising administering a therapeutically effective amount of the antibody or antigen-binding fragment thereof provided herein and/or the pharmaceutical composition provided herein to a subject in need thereof. In certain embodiments, the disease or condition is a SIRPα related disease, disorder or condition.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g. physician or veterinarian) as indicated by these and other circumstances or requirements.

In certain embodiments, the antibody or antigen-binding fragment provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g. a therapeutic response). For example, a single dose may be administered, or several divided doses may be administered over time.

The antibodies or antigen-binding fragments thereof provided herein may be administered by any route known in the art, such as for example parenteral (e.g. subcutaneous, intraperitoneal, intravenous, including intravenous infusion, intramuscular, or intradermal injection) or non-parenteral (e.g. oral, intranasal, intraocular, sublingual, rectal, or topical) routes.

In some embodiments, the antibodies or antigen-binding fragments thereof provided herein may be administered alone or in combination a therapeutically effective amount of an additional therapeutic agent. For example, the antibodies or antigen-binding fragments thereof disclosed herein may be administered in combination with an additional therapeutic agent, for example, a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, or cytokines.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In another aspect, the present disclosure further provides methods of modulating SIRPα activity in SIRPα-positive cells, comprising exposing the SIRPα-positive cells to the antibodies or antigen-binding fragments thereof provided herein. In some embodiments, the SIRPα-positive cell is a phagocytic cell (e.g. a macrophage).

In another aspect, the present disclosure provides methods of detecting the presence or amount of SIRPα in a sample, comprising contacting the sample with the antibody or antigen-binding fragment thereof provided herein, and determining the presence or the amount of SIRPα in the sample.

In another aspect, the present disclosure provides a method of diagnosing a SIRPα related disease, disorder or condition in a subject, comprising: a) contacting a sample obtained from the subject with the antibody or an antigen-binding fragment thereof provided herein; b) determining the presence or amount of SIRPα in the sample; and c) correlating the presence or the amount of SIRPα to existence or status of the SIRPα related disease, disorder or condition in the subject.

In another aspect, the present disclosure provides kits comprising the antibody or antigen-binding fragment thereof provided herein, optionally conjugated with a detectable moiety, which is useful in detecting a SIRPα related disease, disorder or condition. The kits may further comprise instructions for use.

In another aspect, the present disclosure also provides use of the antibody or antigen-binding fragment thereof provided herein in the manufacture of a medicament for treating, preventing or alleviating a SIRPα related disease, disorder or condition in a subject, in the manufacture of a diagnostic reagent for diagnosing a SIRPα related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Reagent Generation

### 1.1 Reference Antibody Generation

The DNA sequences encoding variable regions of anti-SIRPα reference antibodies HEFLB (see US20140242095), or hu1H9G4 (see WO2019/023347A1) were cloned into the vectors expressing human IgG constant regions. The variable region amino acid sequences of HEFLB, and hu1H9G4 are shown in Table 6 of the present disclosure. The expression plasmids transfected Expi293 cells (Invitrogen) were cultured at 37° C. for 5 days. Then the culture medium was collected and centrifuged to remove cell pellets. The harvested supernatant was purified using Protein A affinity chromatography column. HEFLB and hu1H9G4 are both human IgG4 monoclonal antibodies with the S228P mutation in the constant region.

### 1.2. SIRPα, SIRPB and SIRPγ Stable Expression Cell Lines Generation

The DNA sequence encoding full length human SIRPα v1 (NP_542970), human SIRPB (O00241), cyno SIRPα (NP_001271679), or C57BL/6 mouse SIRPα (NP_031573) was cloned into the pIRES vector (Clontech) respectively. Human SIRPγ (Q9P1W8) expression plasmid was purchased from Sino Biological (HG16111-CF).

293F cells (Invitrogen) transfected with human SIRPα v1 or human SIRPγ expression plasmid were selectively cultured and stable clones were obtained and confirmed.

In a similar way, CHOK1 cells (Invitrogen) transfected with human SIRPα v1, human SIRPB, cyno SIRPα or C57BL/6 mouse SIRPα expression plasmid were selectively cultured and stable clones were obtained and confirmed.

CHOK1 cell line stable expressing exogenous human SIRPα v2 (CAA71403.1) was purchased from KYinno (KC-1720).

### 1.3. Recombinant Proteins Generation

Recombinant proteins of human IgG Fc (hFc) tagged human CD47 extracellular domain (ECD, NP_001768.1, M1-E141), human SIRPα v1 ECD (NP_542970, M1-R370), human SIRPα v2 ECD (CAA71403.1, M1-R369), or human SIRPY ECD (Q9P1W8, M1-P360) were generated by Chempartner. Recombinant proteins of 6×His tagged C57BL/6 mouse SIRPα ECD, human SIRPBL ECD (NP_001129316.1) and mouse human IgG Fc (mFc) tagged human CD47 ECD, human SIRPα v1 were purchased from Biointron. Recombinant proteins of 6×His tagged human SIRPα v1 ECD, human SIRPα v2 ECD, human SIRPβ ECD (000241) were purchased from Sino Biological.

### Example 2. Antibody Generation

### 2.1. Preparation of Immunogen for Protein Immunization

hFc tagged human SIRPα v1 ECD recombinant protein was used as immunogen for protein immunization (refer to Example 1.3).

### 2.2. Preparation of Immunogen for Cell Immunization

293F cells stably expressing human SIRPα v1 were used as immunogen for cell immunization (refer to Example 1.2).

### 2.3. Preparation of Immunogen for Genetic Immunization

The DNA sequence encoding full length human SIRPα v1 protein (NP_542970) was cloned into the pCP vector (Chempartner). Then prepared plasmids were coating onto colloidal gold bullets (Bio-Rad) as immunogen for genetic immunization.

### 2.4. Immunization

Balb/c and SJL/J mice (SLAC) were immunized by three different strategies of protein immunization using human SIRPα v1 ECD recombinant protein, cell immunization using 293F cells stably expressing human SIRPα v1 and genetic immunization using gold bullets coated with human SIRPα v1 expression plasmid. ELISA assay with human SIRPα v1 ECD recombinant protein and FACS assay with CHOK 1 cells stably expressing human SIRPα v1 were used to detect serum titer of immunized mice. Mice with high serum titer were selected for hybridoma fusion.

### 2.5. Hybridoma Generation

5 days after final boost, mice were sacrificed and the spleen cells were collected. 1% (v/v) NH₄OH was added to lyse erythrocytes. Then the washed spleen cells were fused with SP2/0 mouse myeloma cells (ATCC) by high-efficiency electro-fusion or PEG method. After cell fusion, the fused cells were seeded into 96-well plates at the density of 2×10⁴ cells/well with 200 μl DMEM medium containing 20% FBS and 1% HAT.

### 2.6. Hybridoma Screening

10⁻¹² days after fusion, fusion plates were primarily screened by ELISA assay with human SIRPα v1 and v2 ECD recombinant proteins or Acumen assay (TTP Labtech) with CHOK1 cells stably expressing human SIRPα v1. The hybridoma cells from positive wells were amplified into 24-well plates for 2nd screening. In 2nd screening, binding activity was assessed by ELISA assay with human SIRPα v1 and v2 ECD recombinant proteins and FACS assay with CHOK1 cells stably expressing human SIRPα v1. Clones with top binding activity against different human SIRPα variants were selected for subcloning. In addition, the specificity against human SIRPα/β/γ, species cross reactivity, CD47 and SIRPα interaction blocking activity were also detected in 2nd screening for hybridoma characterization (refer to Example 3 for methods of the characterization assays).

### 2.7. Hybridoma Subclone

Hybridoma cells of each selected clone were seeded into 96-well plates at the density of 1 cell/well by limiting dilution. The plates were screened by the same way as hybridoma primary screening (refer to Example 2.6). The positive single clones were picked and characterized by the same way as hybridoma 2nd screening (refer to Example 2.6). Then the monoclonal hybridoma cell lines with top binding activity were obtained for further hybridoma antibody production, characterization, and sequencing. A total of 7 antibody clones were identified as functional hits, and the hybridoma antibodies purified from these clones were assigned as 005, 015, 025, 042, 059, 071, and 073 respectively (Example 3).

### Example 3. Antibody Characterization

### 3.1. Hybridoma Antibody Production and Purification

After about 14 days of culturing, the hybridoma cell culture medium was collected and centrifuged to remove cells. After filtered through 0.22 μm PES membrane and adjusting pH to 7.4, the harvested supernatants were loaded to Protein A affinity chromatography column (GE). Antibodies were eluted by 0.1 M citrate sodium buffer (pH3.0) followed by immediately neutralization using Tris buffer (pH8.0). After dialysis with PBS buffer, the antibody concentration was determined by Nano Drop (Thermo Fisher). The purity of proteins was evaluated by SDS-PAGE and HPLC-SEC (Agilent). The endotoxin level was detected with Endochrome-K kit (Charles River).

### 3.2. Binding Specificity Detection

Binding specificity of the purified hybridoma antibodies against human SIRPα variants was detected by ELISA assay using recombinant proteins of Fc tagged human SIRPα v1 ECD and human SIRPα v2 ECD. Briefly the antibodies were incubated with ELISA microplate coated antigens at 37° C. for 1 hour. After washing, horseradish peroxidase (HRP) labeled anti-mouse IgG 2nd Ab (Sigma) was added and incubated at 37° C. for 1 hour. Then, 100 μl/well of TMB solution (Biotechnology) was added. After incubation for 15 minutes at room temperature, the reaction was stopped by the addition of 50 ul of 1N HCl. OD 450 nm was read and EC₅₀ was calculated using GraphPad Prism9.0. The binding specificity property of HEFLB and 7 functional antibodies is summarized in Table 8. Other than HEFLB, all antibodies as tested bind to both human SIRPα v1 and human SIRPα v2. HEFLB can only bind to human SIRPα v1 but not human SIRPα v2.

### 3.3. Species Cross Reactivity Detection

Species cross reactivity of the purified hybridoma antibodies against human, cyno and mouse SIRPα was determined by FACS assay using CHOK1 cells stably expressing human SIRPα v1, CHOK 1-cyno SIRPα and C57BL/6 mouse SIRPα. Briefly the antibodies were incubated with 2×10⁵ target cells at 4° C. for 1 hour. After washing, fluorescence labeled anti-mouse IgG 2^{nd} antibody (Life Technologies) was added and incubated at 4° C. for 1 hour. Geometric median fluorescence intensity was detected and EC₅₀ was calculated using GraphPad Prism9.0. The species cross reactivity property of HEFLB and 7 functional antibodies is summarized in Table 8. Other than HEFLB, all antibodies as tested can bind to cyno SIRPα. None of tested antibodies can bind to C57BL/6 mouse SIRPα.

### 3.4. CD47/SIRPα Interaction Blocking Activity Detection

Competitive ELISA assay was used to determine whether the purified hybridoma antibodies can block CD47 and SIRPα interaction. Briefly, antibody and biotin labeled soluble human SIRPα v1 ECD recombinant protein were co-incubated with ELISA microplate coated human CD47 ECD recombinant protein. After washing, HRP labeled streptavidin (HRP-SA, Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. The blocking ratios were determined by blockade of biotin labeled human SIRPα v1 ECD recombinant protein binding to ELISA microplate coated human CD47 ECD recombinant protein. ICso and top blocking ratio calculated using GraphPad Prism9.0 are summarized in Table 8. Other than 005, all antibodies as tested can block human CD47 and human SIRPα v1 interaction.

### 3.5. Hemagglutination Activity Detection

Anti-CD47 antibodies may promote red blood cell (RBC) hemagglutination, which leads to potential safety risk. The hemagglutination activity of the purified hybridoma antibodies were tested. Briefly, human RBCs were diluted to 10% in PBS and incubated at 37° C. for 1 hour at the presence of 100 nM antibodies. Evidence of hemagglutination is demonstrated by the presence of non-settled RBCs, appearing as a haze compared to punctuate red dot of non-hemagglutinated RBCs. Hemagglutination index was determined by quantitating the area of the RBC pellet in the presence of antibody, normalized to that in the absence of antibody. As summarized in Table 8, all 7 functional antibodies didn't exhibit hemagglutination activity.

### 3.6. SHP-1 Recruitment Detection

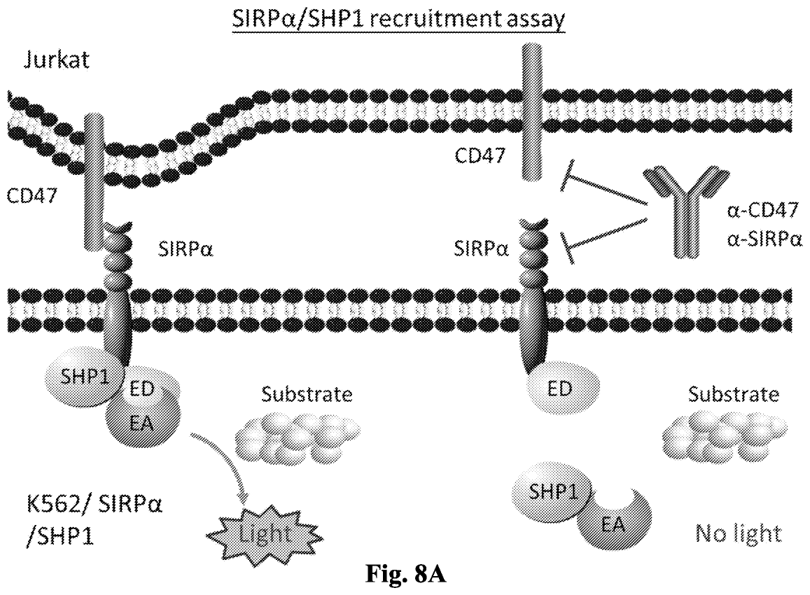

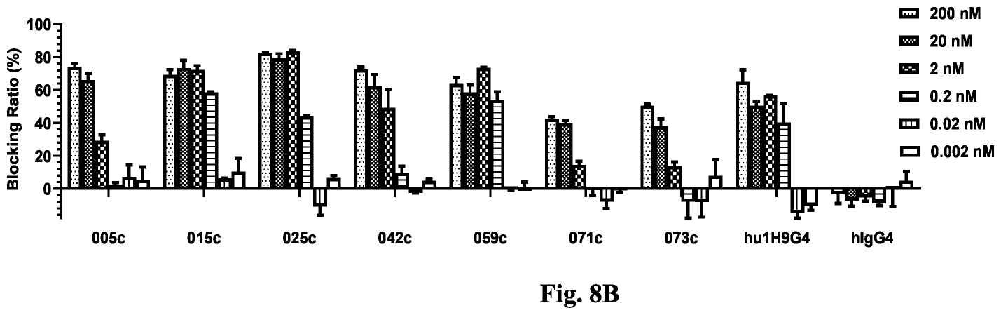

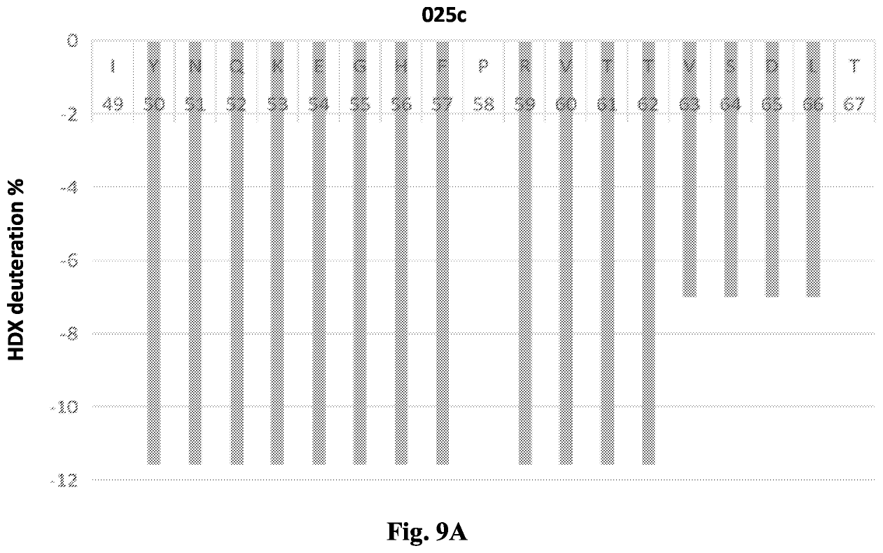

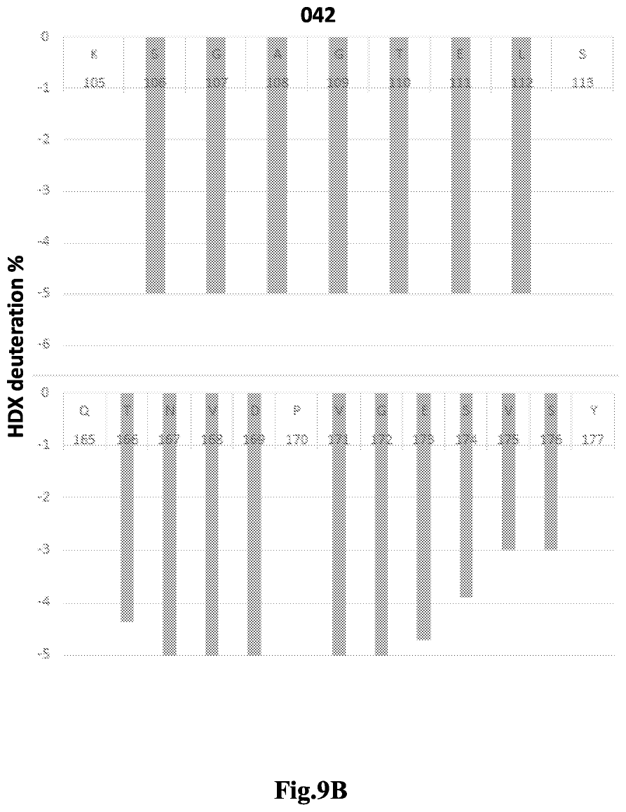

The efficacy of the purified hybridoma antibodies to block CD47/SIRPα mediated “don't eat me” signaling was assessed by cell-based SHP-1 recruitment assay (FIG. 8A). Full length human SIRPα v1 was engineered with a small beta-gal fragment (ED) fused to its C-terminal, and the SH2-domain of SHP-1 was engineered with the complementing beta-gal fragment (EA). These constructs were stably expressed in K562 cells. Ligand engagement, through co-culture with human CD47 expressing cells, results in phosphorylation of SIRPα-ED fusion protein, leading to the recruitment of SHP-1-EA which forces to create an active beta-gal enzyme. This active enzyme hydrolyzes substrate to create chemiluminescence as a measure of reporter activity. The blocking ratios were determined by blockade of beta-gal enzyme activity. As summarized in Table 8, anti-SIRPα hybridoma antibodies 005, 015, 025, 042, 059, 071, and 073 potently disrupted CD47/SIRPα mediated “don't eat me” signaling. These 7 antibodies were considered as functional hits.

### 3.7. Hybridoma Sequencing

Total RNA isolated from monoclonal hybridoma cells was reverse-transcribed into cDNA using either isotype-specific anti-sense primers or universal primers following the technical manual of SMARTScribe Reverse Transcriptase. Then the cDNA was used as template to amplify antibody fragments of heavy chain and light chain according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes and insert fragments were analyzed by DNA sequencing. Finally, the consensus sequences were identified as antibody variable regions of heavy chain and light chain.

### Example 4. Chimeric Antibody Generation and Characterization

### 4.1. Chimeric Antibody Generation and Production

According to hybridoma sequencing results mouse anti-SIRPα functional hits were converted into human IgG4 chimeric antibodies with S228P mutation for characterization. Briefly the DNA sequence encoding heavy chain variable region was cloned into the pcDNA3.4-hIgG4P vector (Biointron) carrying human IgG4 heavy chain constant region with S228P mutation. The DNA sequence encoding light chain variable region was cloned into the pcDNA3.4-hIgGk vector (Biointron) carrying human kappa light chain constant region. Expi293 cells (Life Technologies) co-transfected with antibody heavy and light chain expression plasmids were expanded at 37° C. for 5 days. The resulting chimeric antibodies are referred to herein as 005c, 015c, 025c, 042c, 059c, 071c, and 073c where the suffix “c” indicates chimeric.

### 4.2. Chimeric Antibody Characterization

### 4.2.1 Binding Specificity Detection

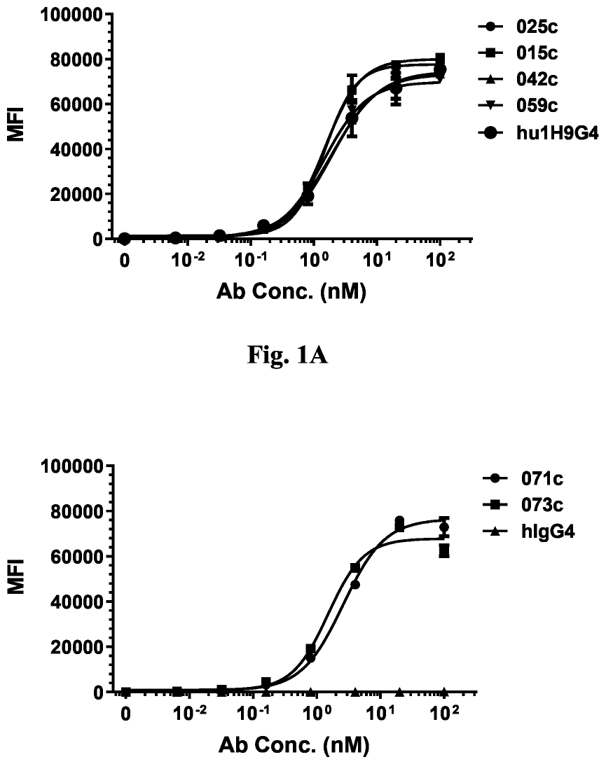

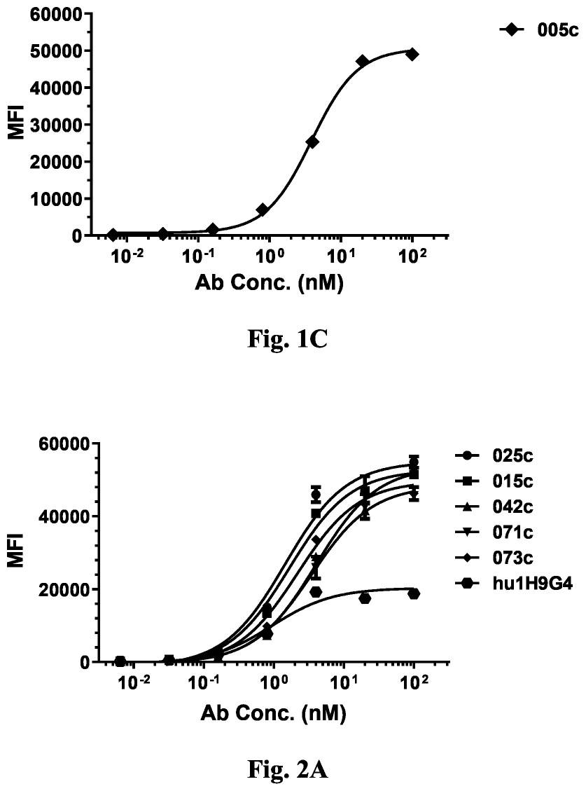

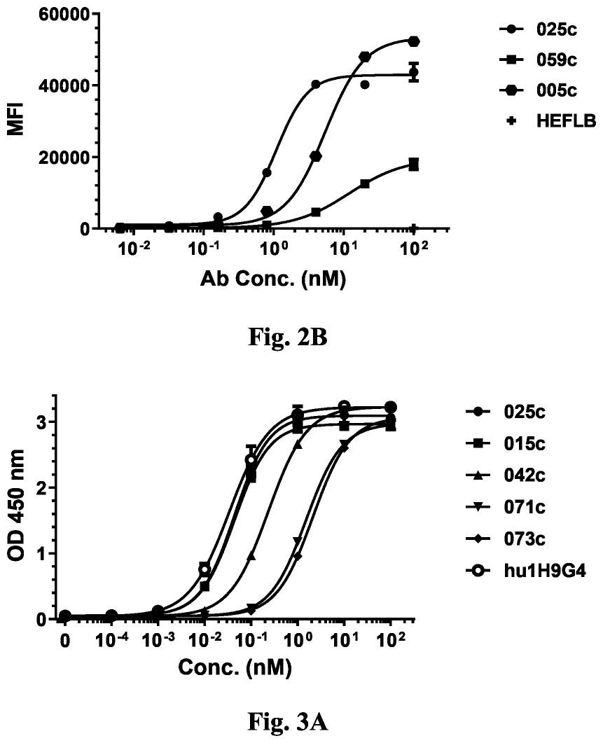

Binding activity of the purified chimeric antibodies against human SIRPα variants was detected by FACS assay using CHOK1 cells (FIGS. 1A and 1B) or 293F cells (FIG. 1C) stably expressing human SIRPα v1 and CHOK1 cells stably expressing human SIRPα v2 (FIGS. 2A and 2B). As shown in FIGS. 1A, 1B, and 1C, all antibodies as tested strongly bind to cell surface human SIRPα v1. As shown in FIG. 2, other than HEFLB, all antibodies as tested bind to cell surface human SIRPα v2. EC₅₀ and top signal calculated using GraphPad Prism9.0 are summarized in Table 9.

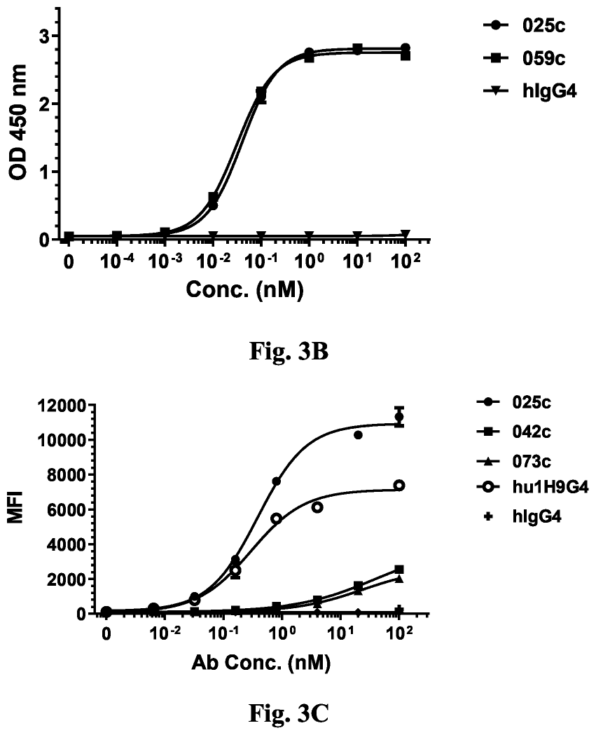

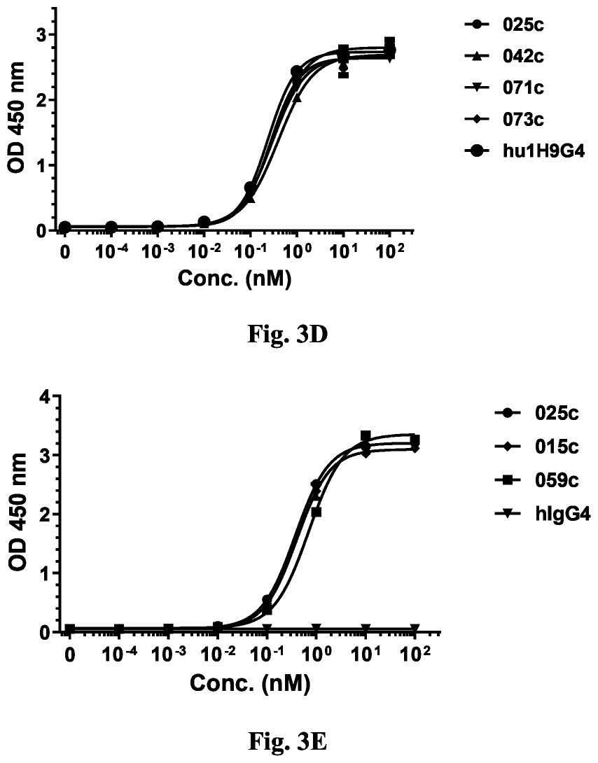

Binding activity of the purified chimeric antibodies against SIRPβ and SIRPBI was detected by ELISA assay using recombinant proteins of human SIRPβ ECD (FIGS. 3A and 3B), human SIRPBI ECD (FIGS. 3D and 3E) and FACS assay using CHOK 1 cells stably expressing human SIRPB (FIG. 3C). As shown in FIG. 3A to 3C, all antibodies as tested bind to human SIRPB at different levels, among which 042c, 071c and 073c have weaker binding. As shown in FIGS. 3D and 3E, all antibodies as tested strongly bind to human SIRPβI. EC₅₀ and top signal calculated using GraphPad Prism9.0 are summarized in Table 10.

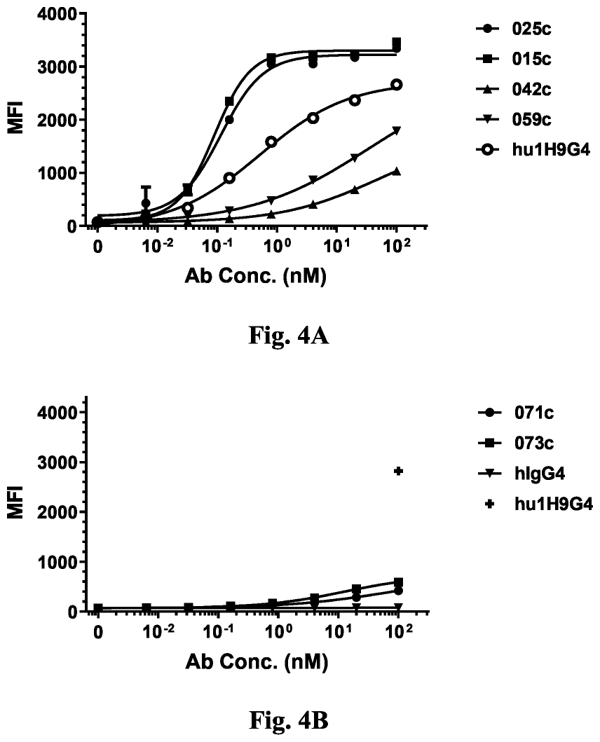

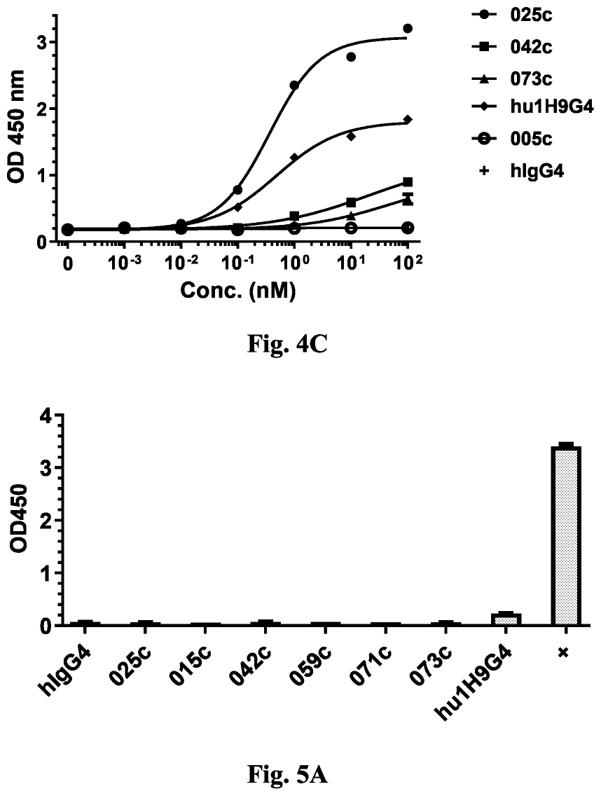

Binding activity of the purified chimeric antibodies against SIRPγ was detected by FACS assay using recombinant protein of cyno SIRPγ ECD (FIG. 4C) and FACS assay using 293F cells stably expressing human SIRPγ (FIGS. 4A and 4B). As shown in FIGS. 4A and 4B, all antibodies as tested bind to human SIRPγ at different levels, among which 042c, 059c, 071c and 073c have very weak binding. As shown in FIG. 4C, 005c, 042c and 073c have very weak binding to cyno SIRPγ, which is correlated with their binding activity to human SIRPγ. EC₅₀ and top signal calculated using GraphPad Prism9.0 are summarized in Table 11.

### 4.2.2 Species Cross Reactivity Detection

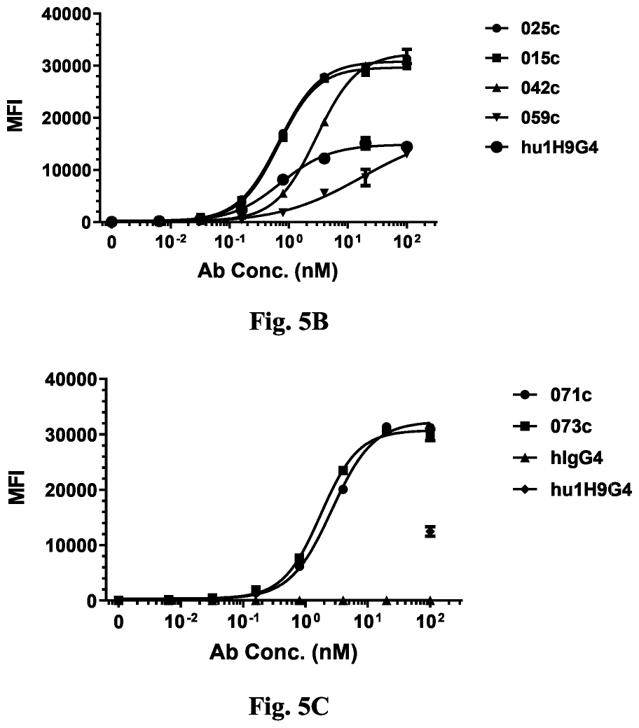

Species cross reactivity of purified chimeric antibodies was detected by ELISA assay using recombinant protein of C57BL/6 mouse SIRPα ECD (FIG. 5A) and FACS assay using CHOK 1 cells stably expressing cyno SIRPα (FIGS. 5B and 5C). All antibodies as tested bind to cyno SIRPα at different levels but have no species cross reactivity against C57BL/6 mouse SIRPα. EC₅₀ and top signal calculated using GraphPad Prism9.0 are summarized in Table 12.

### 4.2.3. CD47/SIRPα Interaction Blocking Activity Detection

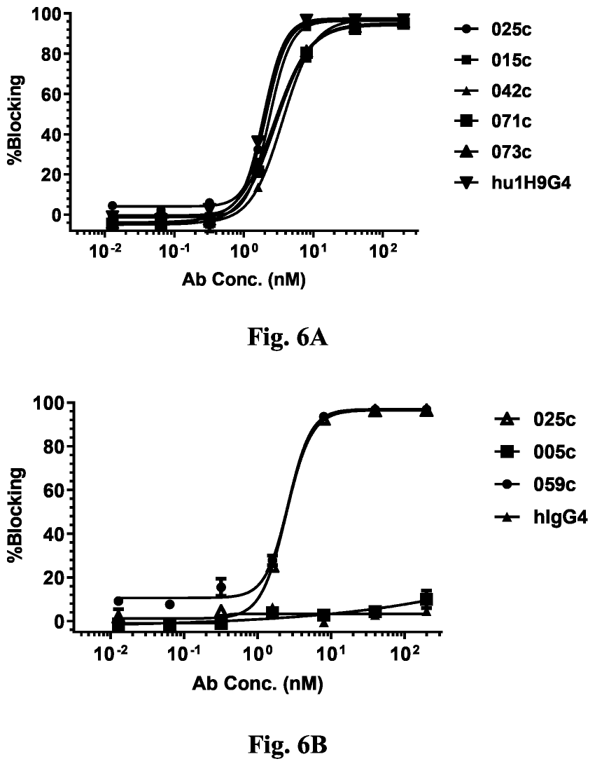

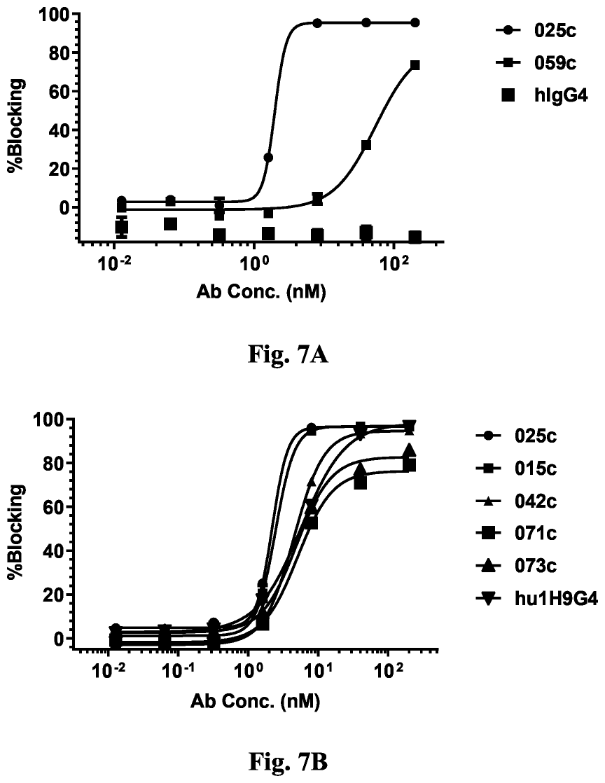

Competitive ELISA assay was used to determine whether the purified chimeric antibodies can block CD47 and SIRPα interaction. Briefly, antibody and mFc tagged human CD47 ECD recombinant protein were co-incubated with ELISA microplate coated human SIRPα v1 ECD (FIGS. 6A and 6B) or human SIRPα v2 ECD (FIGS. 7A and 7B) recombinant protein. After washing, HRP labeled anti-mouse Fc 2nd antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Blocking ratio was determined by blockade of human CD47 ECD recombinant protein binding to ELISA microplate coated human SIRPα ECD recombinant protein. ICso and top blocking ratio calculated using GraphPad Prism9.0 are summarized in Table 13. Other than 005, all antibodies as tested can block interaction between human CD47 and different human SIRPα variants.

### 4.2.4. SHP-1 Recruitment Assay

The efficacy of the purified chimeric antibodies to block CD47/SIRPα mediated “don't eat me” signaling was assessed by cell-based SHP-1 recruitment assay (FIG. 8B, refer to methods described in Example 3.6). ICso and top blocking ratio were calculated using GraphPad Prism9.0. As summarized in Table 14, all antibodies as tested can disrupt CD47/SIRPα mediated “don't eat me” signaling at different levels. In particular, although 005c doesn't block CD47 and SIRPα interaction, it can inhibit CD47 engagement resulted SHP-1 recruitment to SIRPα intracellular tail.

### 4.2.5. Affinity Detection

The purified chimeric antibodies were characterized for binding affinity against human SIRPα v1, human SIRPα v2 using Bio-Layer Interferometry technology (Octet system). The association and dissociation curves were fit with 1:1 binding model, and the Ka/Kd/KD values for each antibody were calculated. The affinity data of Ka/Kd/KD values for each antibody are summarized in Table 15.

### 4.2.6. Epitope Analysis

Competitive ELISA assay was used for epitope binning of purified chimeric antibodies. Briefly excessive competitor antibody and mFc tagged human SIRPα v1 ECD recombinant protein were co-incubated with ELISA microplate coated antibody. After washing, HRP labeled anti-mouse Fc 2nd antibody (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Competition ratio was calculated. The antibodies that can compete each other for binding to SIRPα may have related binding epitopes. As shown in Table 16, 025c didn't show competitive binding to human SIRPα with 042c, 073c and hu1H9G4, indicating that it may bind to a distinct epitope. Competition between 042c, 073c and hu1H9G4 is not bidirectional, indicating that their binding epitopes may be related but not completely identical.

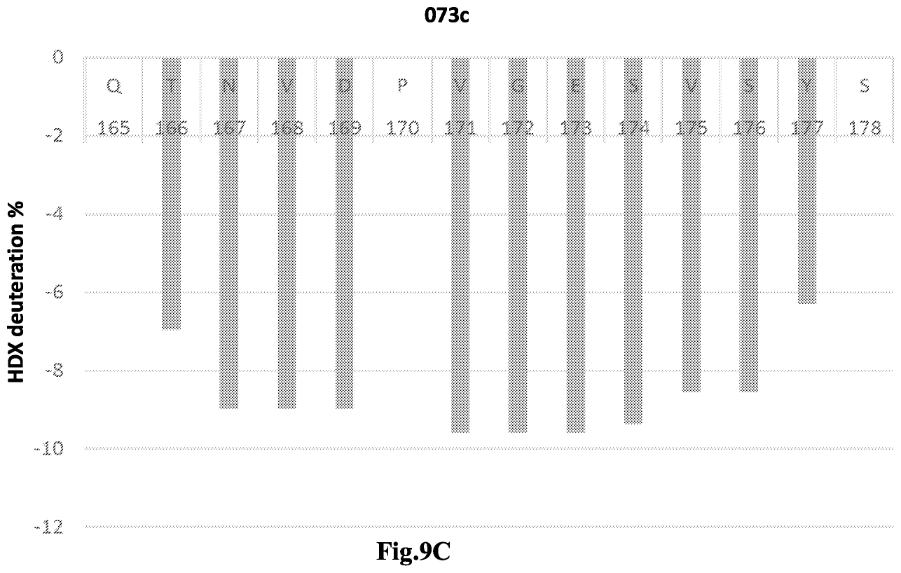

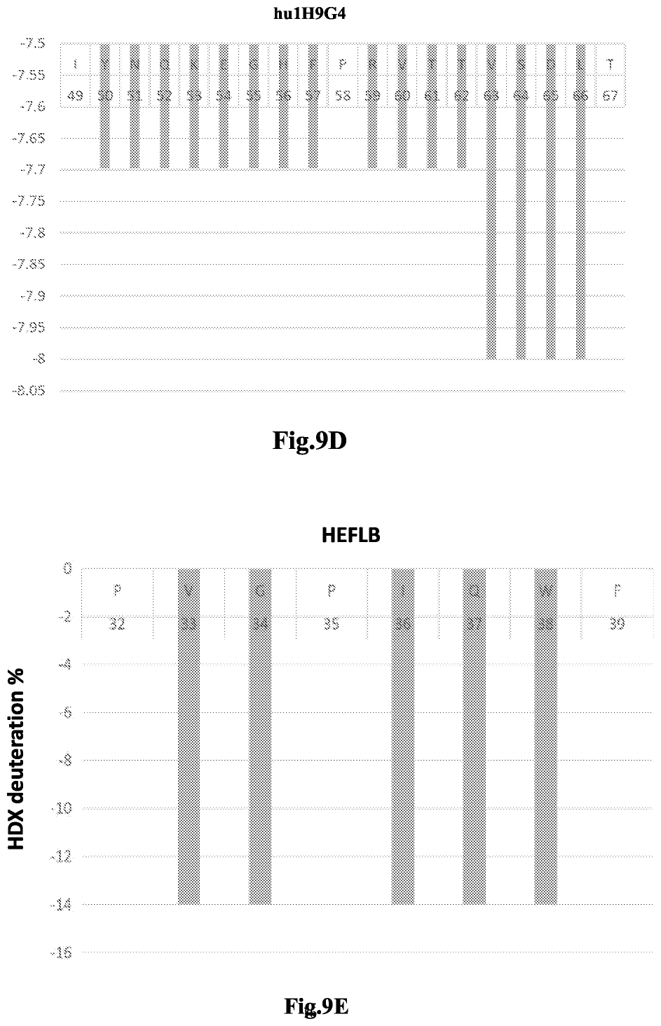

Epitope mapping of 025c, 042c, 073c, HEFLB and hu1H9G4 were further carried out using hydrogen deuterium exchange mass spectrometry (HDX-MS). As shown in FIG. 9A, 025c binding resulted in less hydrogen deuterium exchange ratio of the region of YNQKEGHFPRVTTVSDL of His tagged human SIRPα v1 ECD, indicating these amino acids may be critical for 025c to bind. As shown in FIG. 9B, 042c binding resulted in less hydrogen deuterium exchange ratio of 2 regions of SGAGTEL and TNVDPVGESVS of His tagged human SIRPα v1 ECD, indicating these amino acids may be critical for 042c to bind. As shown in FIG. 9C, 073c binding resulted in less hydrogen deuterium exchange ratio of the region of TNVDPVGESVSY of His tagged human SIRPα v1 ECD, indicating these amino acids may be critical for 073c to bind. In particular, these 3 regions are not located in IgV domain of SIRPα ECD where CD47 binds to, indicating 042c and 073c may work as allosteric antibody to block interaction of CD47 and SIRPα or blocking activity of 042c and 073c is steric hindrance effect. As shown in FIG. 9D, hu1H9G4 binding resulted in less hydrogen deuterium exchange ratio of the region of YNQKEGHFPRVTTVSDL of His tagged human SIRPα v1 ECD, indicating these amino acids may be critical for hu1H9G4 to bind. As shown in FIG. 9E, HEFLB binding resulted in less hydrogen deuterium exchange ratio of the region of VGPIQW of his tagged human SIRPα v1 ECD, indicating these amino acids may be critical for HEFLB to bind.

Taking competitive ELISA data and HDX-MS data together, it is concluded 025c, 042c and 073c may have distinct binding epitopes, which are also different from reference antibodies hu1H9G4 and HEFLB.

### 4.2.7. In Vitro Phagocytosis Assay

The function efficacy of the purified chimeric antibodies was assessed by a flow cytometry based phagocytosis assay. Briefly, M0 nonpolarized or M1 polarized human monocyte derived macrophages with different SIRPA genotypes were co-cultured with CellTrace Violet (Life Technologies) labeled CD47 expressing cancer cells in the presence of antibodies as tested. Phagocytosis was assayed by determining the percentage of macrophages positive for cell trace violet dye. For nonpolarized macrophages, peripheral blood mononuclear cells were seeded into 10 cm tissue culture plates in 1640 supplemented with 10% FBS and 50 ng/ml M-CSF for seven to nine days. Adherent cells were harvested as M0 nonpolarized macrophages. For M1 polarized macrophages, peripheral blood mononuclear cells were seeded into 10 cm tissue culture plates in 1640 supplemented with 10% FBS and 50 ng/ml GM-CSF for 5 days. 50 μg/ml IFNγ and 100 μg/ml LPS were added for additional two to four days culture. Adherent cells were harvested as M1 polarized macrophages.

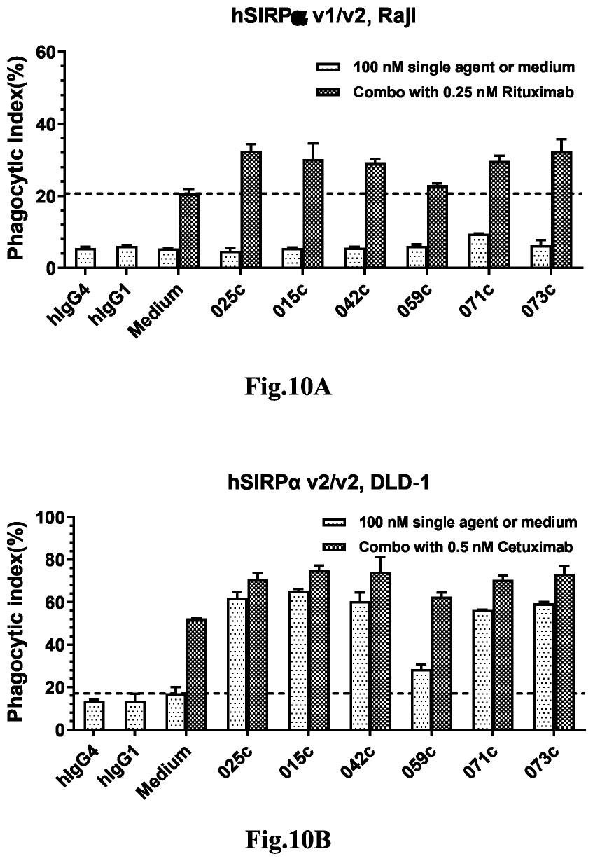

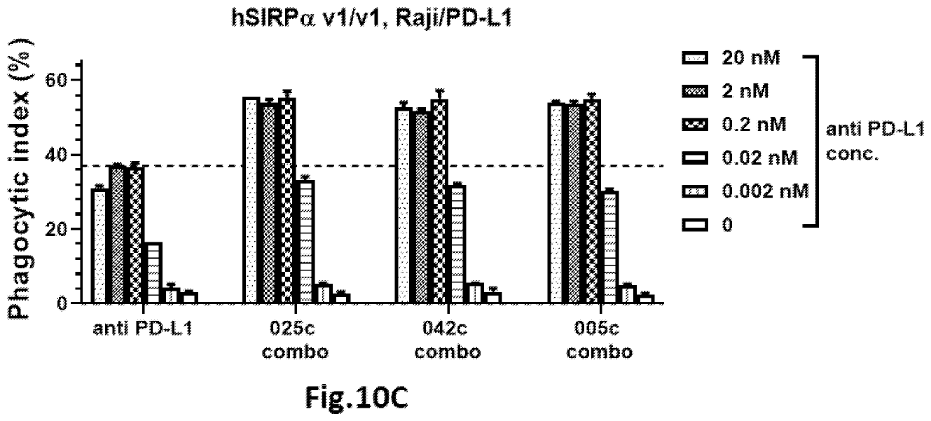

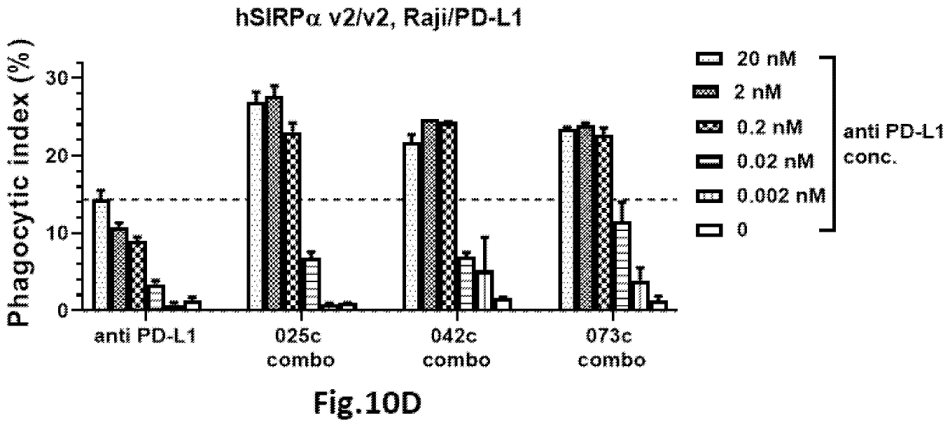

As shown in FIG. 10A, 015c, 025c, 042c, 059c, 071c and 073c showed no single agent activity to enhance tumor cell uptake of Raji cells by M0 macrophages obtained from SIRPA heterozygous v1/v2 individual. However, in the presence of Rituximab (an anti-CD20 antibody), other than 059c that has weaker activity to block interaction between human CD47 and human SIRPα v2, all the other purified chimeric antibodies as tested potentiated macrophage mediated antibody dependent cellular phagocytosis (ADCP) of Raji cells.

As shown in FIG. 10B, other than 059c, regardless of presence of Cetuximab (an anti-EGFR antibody), all the other purified antibodies as tested effectively enhanced tumor cell uptake of DLD-1 cells by M0 macrophages obtained from SIRPA homozygous v2/v2 individual.

Combination of SIRPα antibody plus PD-L1 antibody was tested in phagocytosis assay using M0 macrophages obtained from SIRPA homozygous v1/v1 (FIG. 10C) or v2/v2 (FIG. 10D) individuals. In the presence of PD-L1 antibody, 005c, 025c, 042c and 073c effectively potentiated macrophage mediated ADCP of Raji cells stably expressing PD-L1.

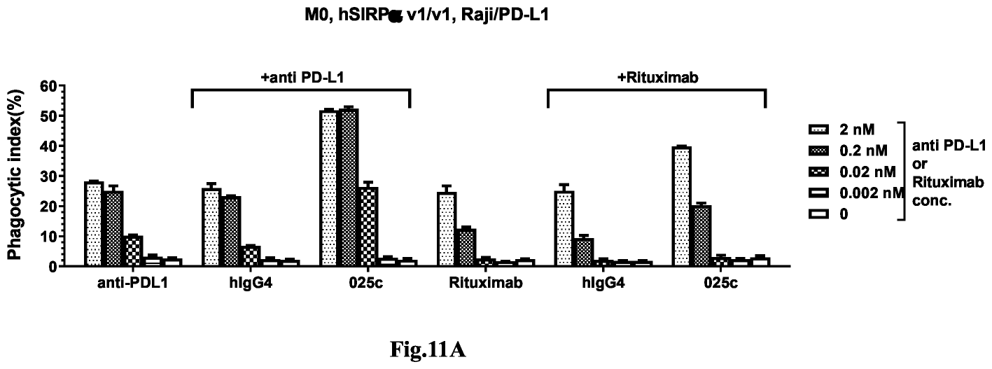

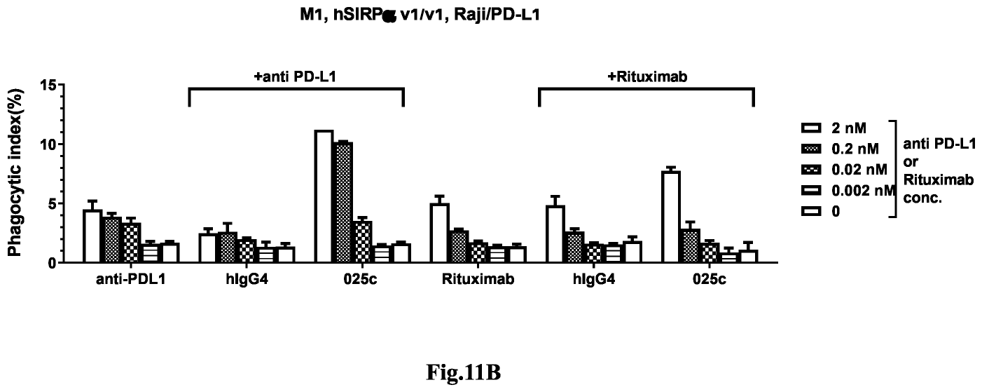

Combinations of 025c plus PD-L1 antibody C71 and 025c plus Rituximab were also tested in phagocytosis using M0 nonpolarized or M1 polarized macrophages obtained from SIRPA homozygous v1/v1 individual. M1 polarized macrophages (FIG. 11B) showed weaker phagocytic capability compared with M0 nonpolarized macrophages (FIG. 11A). Regardless of macrophage polarization status, in the presence of PD-L1 antibody or Rituximab, 025c effectively potentiated macrophage mediated ADCP of Raji cells stably expressing PD-L1. The PD-L1 heavy chain antibody C71 has VH amino acid sequences shown below:

anti-PD-L1 heavy chain antibody C71.VH, SEQ ID
NO: 91:
EVQVVESGGGLVQSGGSLKLSCAGSGFTESAGEMVWHRQVPGKERELVAL
IATPSGSTNYADSVKGRFTISRDNGKNTVYLOMNSLKPEDTAVYYCNIRG
YWGQGTLVTVSS

These data suggest that the antibodies or antigen-binding fragment thereof provided herein potentiate macrophage mediated ADCP of certain tumor cells, when used in combination with an antibody specific for the target antigen of such tumor cells.

### 4.2.8. In Vivo Anti-Tumor Activity

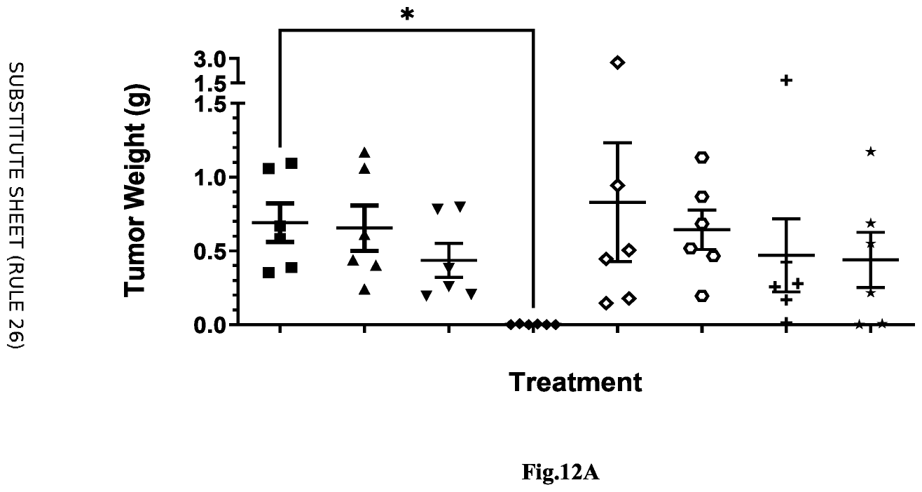

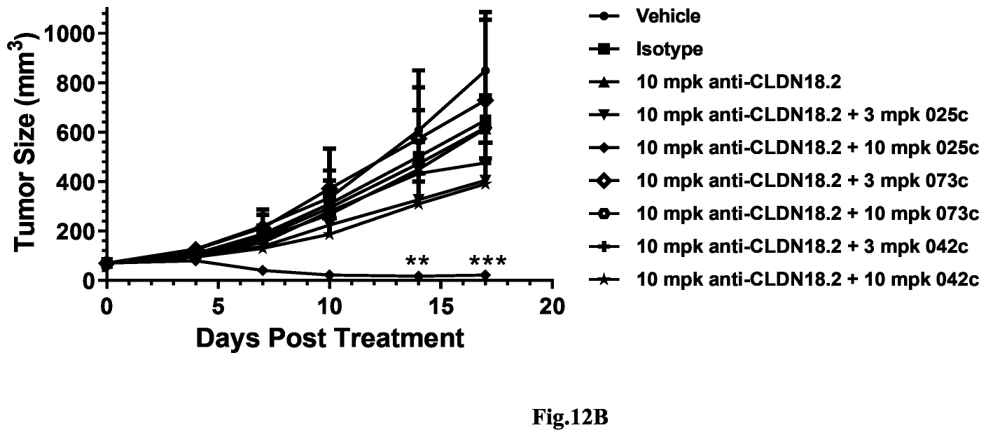

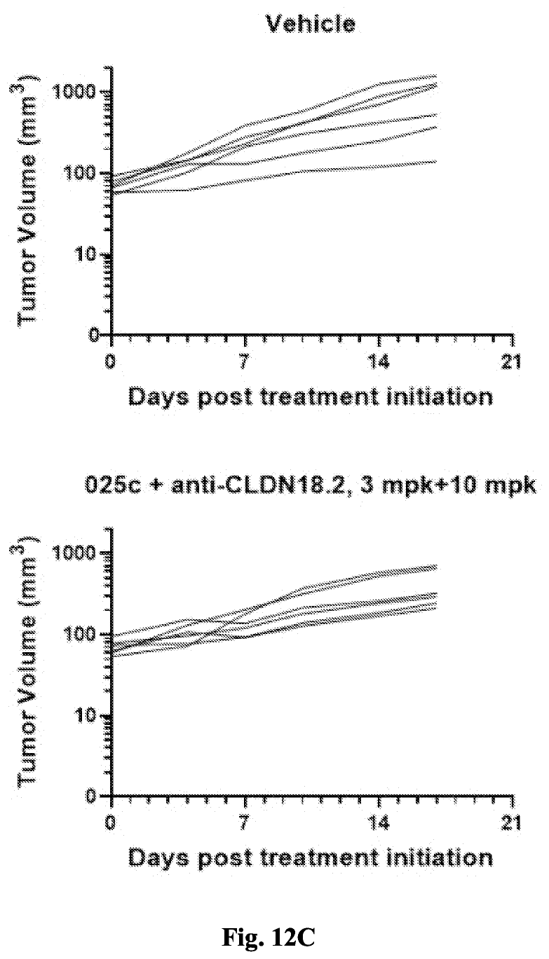

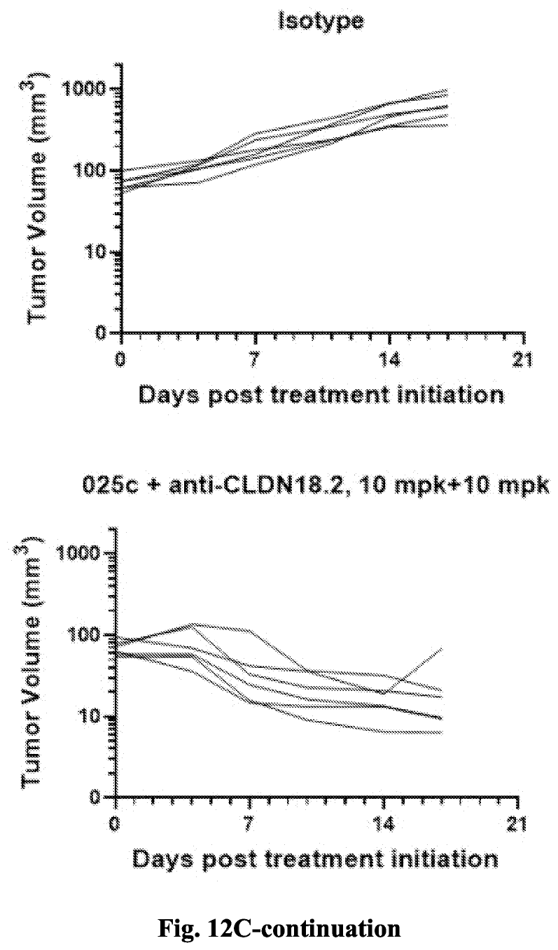

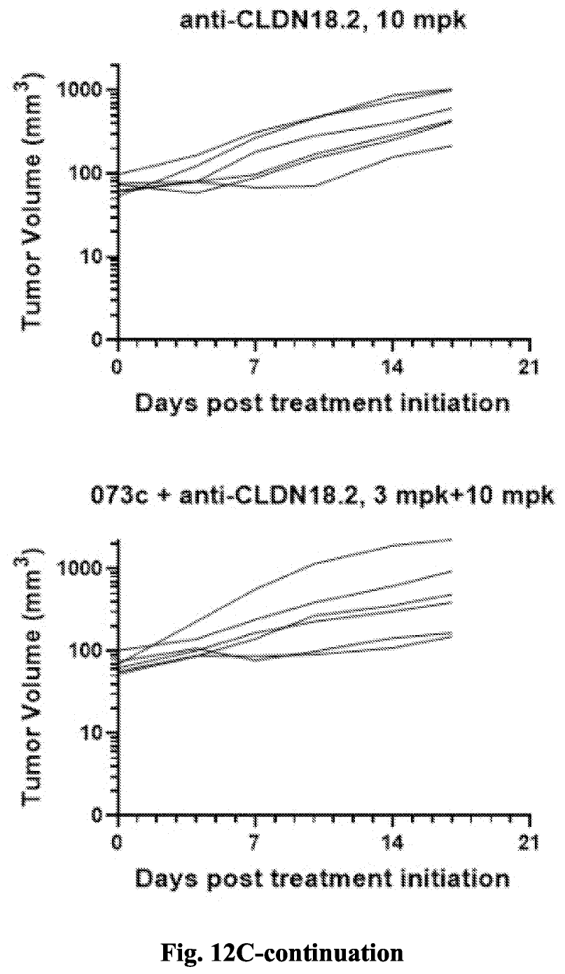

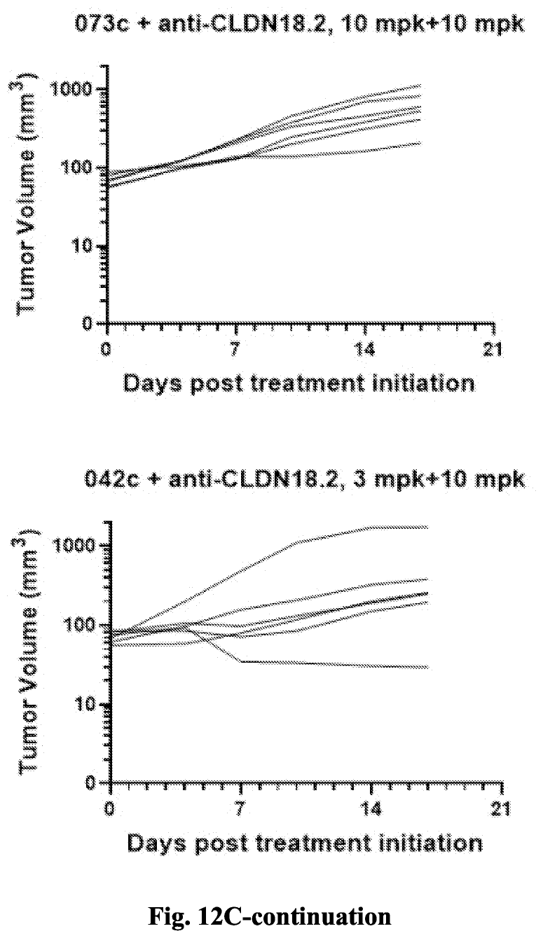

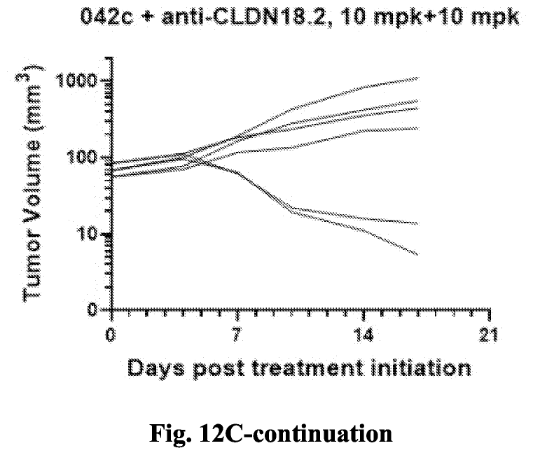

Human CD47/human SIRPα double knock-in mice were inoculated with MC38 cells stably expressing human CD47 and human Claudin18.2 (CLDN18.2). Treatment groups includes vehicle (PBS), isotype control, 10 mg per kg (mpk) anti-CLDN18.2 mAb (22E12) and combination of 10 mpk anti-CLDN18.2 mAb (22E12) plus 3 or 10 mpk anti-SIRPα mAb. Treatment was initiated when tumors reached an average volume of 70-75 mm³. Mice were dosed intraperitoneally (IP) twice a week for 5 times. Tumor volume was measured twice per week. 3 days post the final dosing, mice were sacrificed and tumors were weighted. Statistics were carried out by one-way or two-way anova comparing the mean tumor weight/volume of different treatment groups to that of isotype control group. As shown in FIGS. 12A and 12B, combination of 10 mpk anti-CLDN18.2 mAb (22E12) plus 10 mpk anti-SIRPα significantly inhibited MC38 tumor growth. 6 of 6 tumors in 10 mpk 025c combination group, 1 of 6 tumors in 3 mpk 042c combination group, and 2 of 6 tumors in 10 mpk 042c combination group shrank (FIG. 12C). The VH and VL amino acid sequences of the anti-CLDN18.2 mAb (22E12) are shown below:

anti-CLDN18.2 mAb (22E12) VH,
SEQ ID NO: 88
QVQLVQSGAEVKKPGASVKVSCKASGYTFTNWVHWVRQAPGQGLEWMGE
INPTNARSNYNEKFKKRVTMTRDTSTSTVYMELSSLRSEDTAVYYCARI
YYGNSFAHWGQGTLVTVSS
anti-CLDN18.2 mAb (22E12) VL,
SEQ ID NO: 89
DIVMTQSPDSLAVSLGERATINCKSSQSLLNAGNQKNYLTWYQQKPGQP
PKLLIYWSSTRESGVPDRFSGSGSGTDFTLTISSLQAEDVAVYYCQNNY
YYPLTFGGGTKLEIK

### 4.2.9. Mixed Lymphocyte Reaction Assay (MLR)

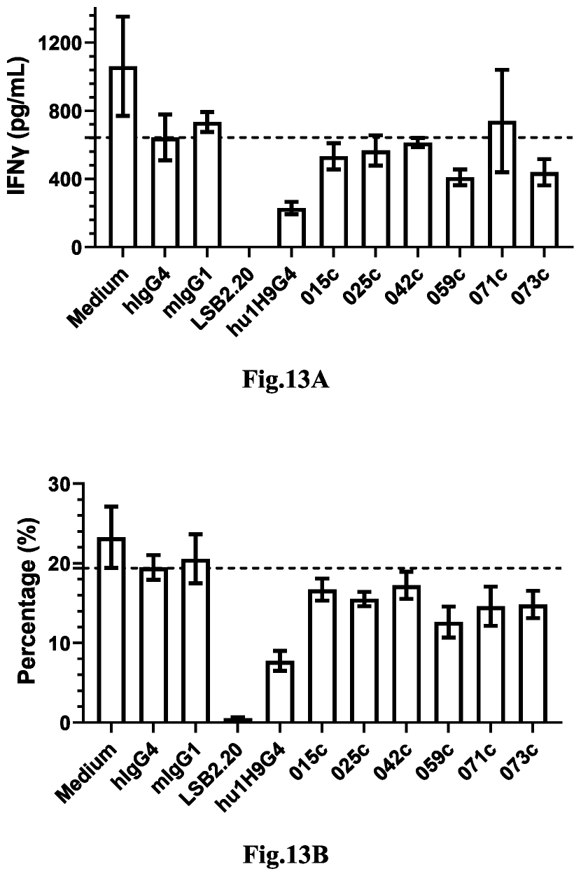

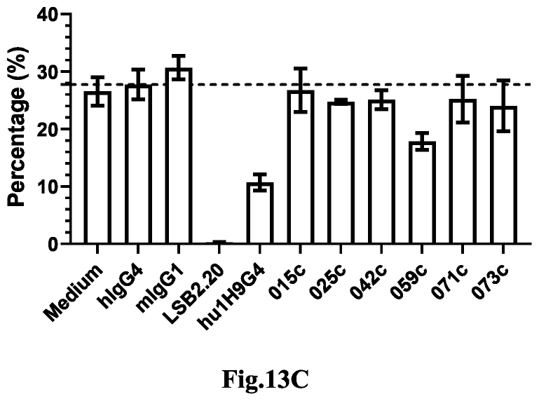

It was reported that adhesion of human T cells to antigen-presenting cells through SIRPγ-CD47 interaction co-stimulates T cell proliferation. Since some of the purified chimeric antibodies strongly bind to human SIRPγ (FIG. 4), to exclude the possibility of interrupting T cell proliferation and activation, the purified chimeric antibodies were tested in MLR assay. Briefly, CellTrace Violet labeled human primary T cells were stimulated with allogeneic mature dendritic cells generated in vitro for 5 days. The indicated antibodies were added from the beginning of the test at a saturating concentration (100 nM). CellTrace Violet low staining was used to determine proliferation population. IFNγ secretion was determined with human IFN gamma kit (Cisbio). As shown in FIG. 13, regardless of binding activity to human SIRPγ, 015c, 025c, 042c, 059c, 071c and 073c showed no significant impact on IFNγ secretion (FIG. 13A), CD4⁺ T cell proliferation (FIG. 13B), and CD8⁺ T cell proliferation (FIG. 13C). As expected, the anti-SIRPγ antibody LSB2.20 (Biolegend) is a potent inhibitor of T cell activation. In particular, hu1H9G4 showed obvious inhibition of IFNγ secretion and T cell proliferation in this assay.

### Example 5. Antibody Humanization

### 5.1. Humanization

CDR grafting method was used for humanization of 025c. Briefly, IGHV1-69-2*01 and IGKV3-11*01 were first selected as humanization templates for heavy and light chain respectively, based on their homology to the original mouse antibody sequences. CDRs were then defined using Kabat definition except heavy chain CDR1, which was defined using a combination of Kabat and Chothia systems. For grafting, the potential hotspots removed CDRs and different combinations of canonical residues from 025c were grafted onto the templates and the resulting variants (human IgG4 antibodies with S228P mutation in the constant region) were expressed via a 96-well high-throughput protein expression system. All the variants produced were tested with FACS assays to select the top binders to human SIRPα v1 and human SIRPα v2 for further characterization. The obtained humanized antibodies with the best binding activity are designated as hu025.021, hu025.023, hu025.033, hu025.059 and hu025.060, where the prefix “hu” indicates “humanized”, and the number in the suffix denotes the serial number of the humanized antibody.

### 5.2. Characterization of Humanized Antibodies

### 5.2.1. Binding Specificity Detection

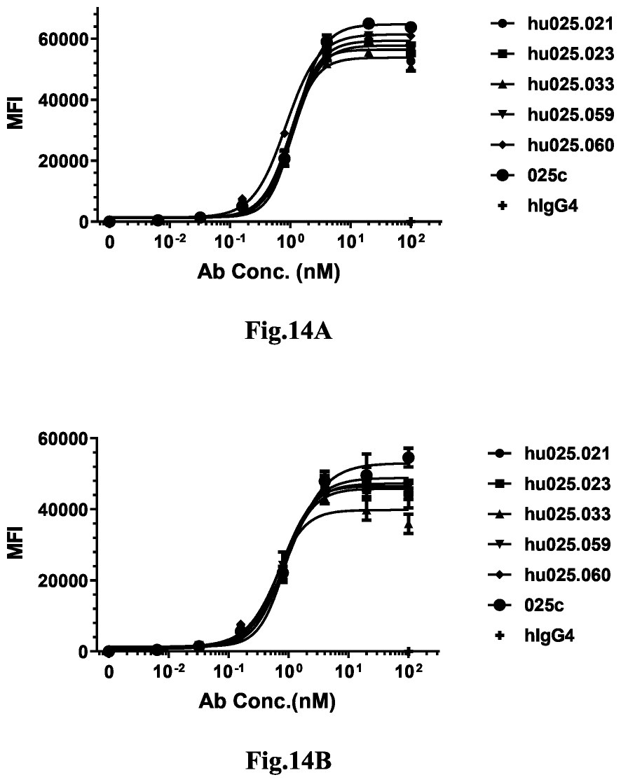

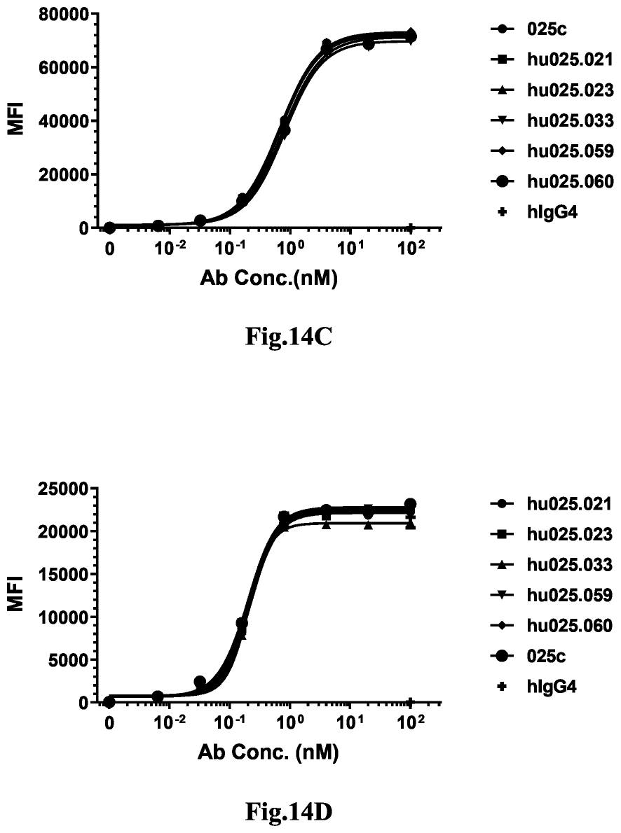

Binding activity of the humanized antibodies against human SIRPα variants was detected by FACS assay using CHOK1 cells stably expressing human SIRPα v1 (FIG. 14A), human SIRPα v2 (FIG. 14B), or human SIRPB (FIG. 14C) and 293F cells stably expressing human SIRPγ (FIG. 14D). All the humanized antibodies as tested were confirmed to retain the similar activity as the parental antibody of 025c to bind to SIRP family members. EC₅₀ and top signal calculated using GraphPad Prism9.0 are summarized in Table 17.

### 5.2.2. CD47/SIRPα Interaction Blocking Activity Detection

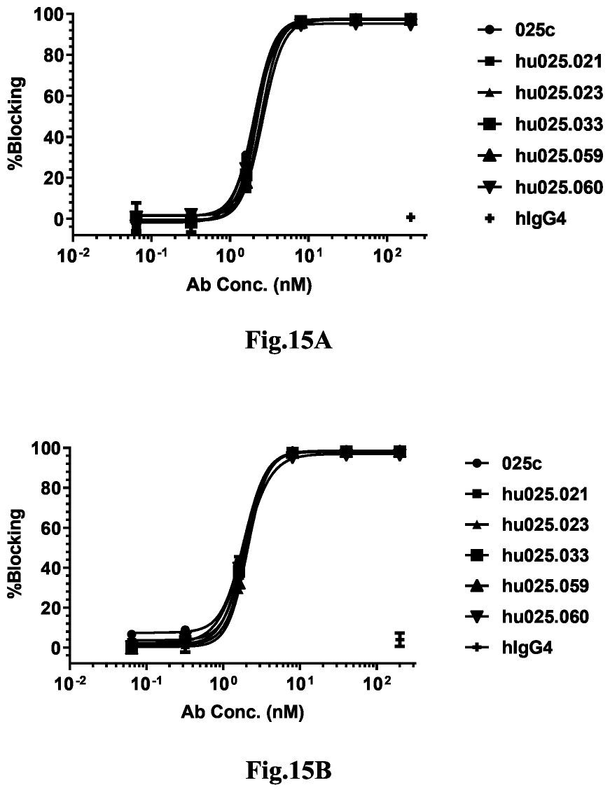

The humanized antibodies were tested for the ability to block CD47 and SIRPα interaction with competitive ELISA assay (refer to methods described in Example 4.2.3.). As shown in FIG. 15, all the humanized antibodies as tested were confirmed to retain the similar activity as parental antibody of 025c to block interaction between human CD47 and different human SIRPα variants. ICso and top blocking ratio calculated using GraphPad Prism9.0 are summarized in Table 18.

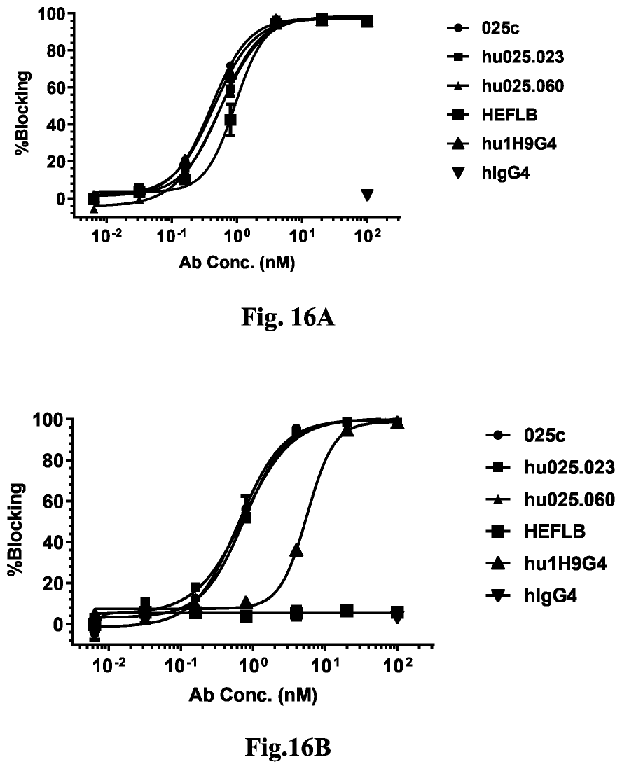

Competitive FACS assay was also set up to further compare the blocking activity of the humanized antibodies and reference antibodies. Briefly antibody and mFc tagged human CD47 ECD recombinant protein were co-incubated with CHOK1 cells stably expressing human SIRPα v1 (FIG. 16A) or human SIRPα v2 (FIG. 16B). After washing, dye labeled anti-mouse Fc 2nd antibody (Sigma) was added and incubated at 37° C. for 1 hour. Fluorescence intensity was detected. Blocking ratio was determined by blockade of human CD47 ECD recombinant protein binding to SIRPα expressed CHOK1 cells. Hu1H9G4 showed weaker activity to block human CD47 and human SIRPα v2 interaction. In particular, HEFLB doesn't work at all for human SIRPα v2. IC₅₀ and top blocking ratio calculated using GraphPad Prism9.0 are summarized in Table 19.

### 5.2.4. SHP-1 Recruitment Assay

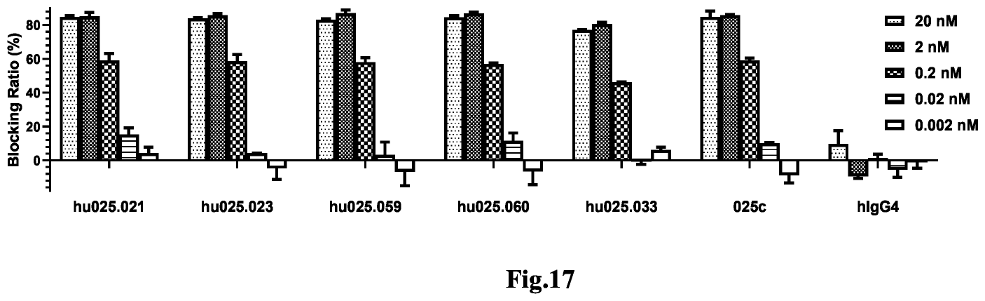

The efficacy of the humanized antibodies to block CD47/SIRPα mediated “don't eat me” signaling was assessed by cell-based SHP-1 recruitment assay (FIG. 17, refer to methods described in Example 3.6). All the humanized antibodies as tested were confirmed to retain the similar activity as the parental antibody of 025c to block CD47 engagement resulted SHP-1 recruitment to SIRPα intracellular tail. IC₅₀ and top blocking ratio calculated using GraphPad Prism9.0 are summarized in Table 20.

### 5.2.5. Affinity Detection

The humanized antibodies were characterized for binding affinity against human SIRPα v1, human SIRPα v2 using Surface Plasmon Resonance technology (Biacore system). The association and dissociation curves were fit with 1:1 binding model, and the Ka/Kd/KD values for each antibody were calculated. The affinity data of Ka/Kd/KD values for each antibody are summarized in Table 21.

### 5.2.6. In Vitro Phagocytosis Assay

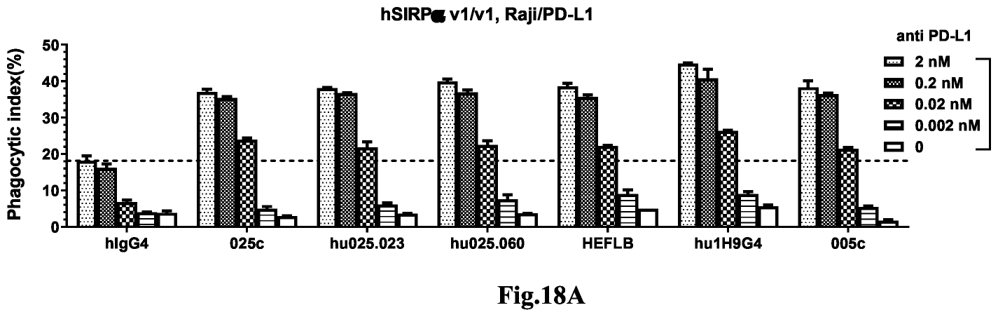

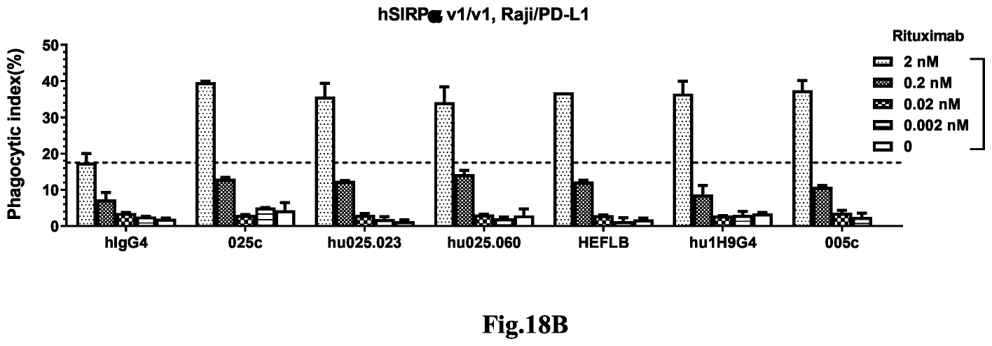

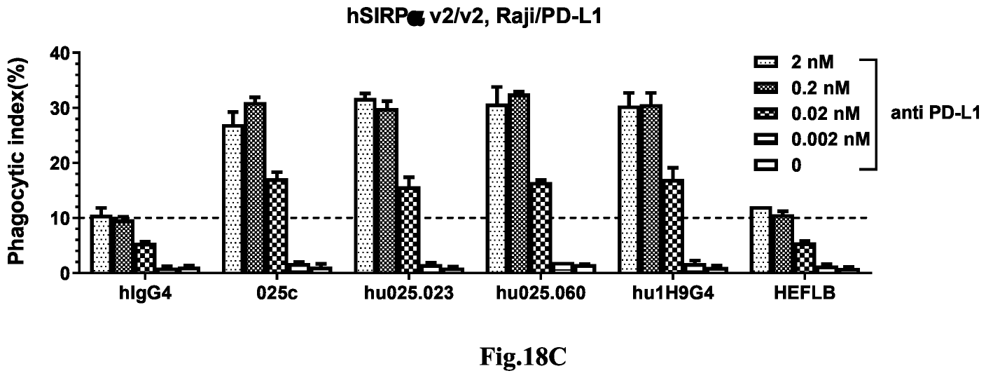

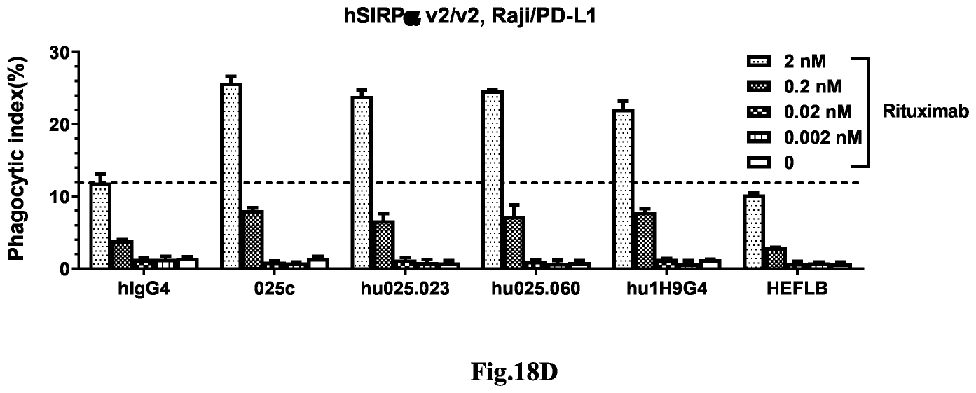

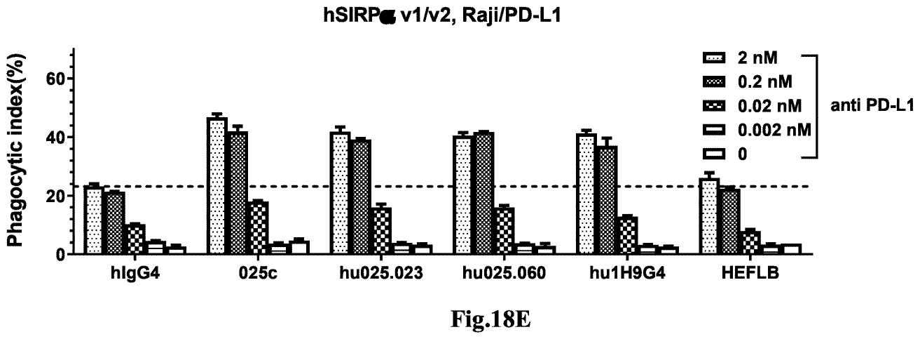

For in vitro function validation, combination of SIRPα antibody plus PD-L1 antibody or Rituximab were tested in phagocytosis assay using M0 macrophages obtained from SIRP A homozygous v1/v1 (FIGS. 18A and 18B), v2/v2 (FIGS. 18C and 18D) or heterozygous v1/v2 individuals (refer to methods described in Example 4.2.7). All the humanized antibodies as tested were confirmed to retain the similar activity as the parental antibody of 025c to potentiate macrophage mediated ADCP of Raji cells stably expressing PD-L1 in the presence of PD-L1 antibody or Rituximab. Reference antibodies and 005c were also tested side by side in these assays. As shown in FIGS. 18A and 18B, 005c, which can block CD47 engagement resulted SHP-1 recruitment to SIRPα intracellular tail but not CD47 and SIRPα interaction, effectively potentiated macrophage mediated ADCP of Raji cells stably expressing PD-L1 in the presence of PD-L1 antibody or Rituximab. As shown in FIGS. 18C, 18D and 18E, HEFLB, which can't bind to human SIRPα v2, didn't work at all for macrophages obtained from SIRPA homozygous v2/v2 and heterozygous v1/v2 individuals.

TABLE 8
anti-SIRPα hybridoma antibodies characterization summary

| | FACS | | | | | | | | |
|---|---|---|---|---|---|---|---|---|---|
| | CHOK1-human | CHOK1-cyno | CHOK1-C57BL/6 | | | hCD47/hSIRPα v1 | | SHP-1 recruitment | |
| | SIRPα v1 (EC₅₀, | SIRPα | mouse SIRPα | ELISA (EC₅₀, nM) | | interaction blocking | | (% blocking) | |
| Antibody | nM) | (MFI at 10 nM) | (MFI at 100 nM) | α V1 | α V2 | (IC₅₀, nM) | Hemagglutination | Test I | Test II |
| 005 | 1.8 | 5972 | − | 0.13 | 0.12 | − | − | 71.02 | 82.65 |
| 015 | 1 | 10823 | − | 0.1 | 0.15 | 0.3 | − | 83.59 | 89.61 |
| 025 | 0.9 | 8561 | − | 0.04 | 0.06 | 0.15 | − | 83.92 | 91.21 |
| 042 | 0.2 | 6848 | − | 0.03 | 0.03 | 0.14 | − | 84.83 | 76.11 |
| 059 | 0.1 | 5843 | − | 0.03 | 0.1 | 0.12 | − | 77.53 | 80.22 |
| 07 | 0.2 | 8371 | − | 0.03 | 0.02 | 0.17 | − | 77.22 | 72.57 |
| 073 | 0.2 | 7850 | − | 0.03 | 0.03 | 0.16 | − | 76.93 | 75.93 |
| HEFLB | 11.2 | − | − | 0.17 | − | 0.5 | − | 46.80 | 50.30 |

Minus symbol stands for no specific signal or no activity.

TABLE 9
binding of anti-SIRPα chimeric antibodies to human
SIRPα v1 and human SIRPα v2

| | |
|---|---|
| 293F-hSIRPα v1 | CHOK1-hSIRPα v2 |

| Ab | EC₅₀ (nM) | TOP MFI | EC₅₀ (nM) | TOP MFI |
|---|---|---|---|---|
| 005c | 3.88 | 49859 | 5.47 | 53333 |

| | |
|---|---|
| CHOK1-hSIRPα v1 | CHOK1-hSIRPα v2 |

| Ab | EC₅₀ (nM) | TOP MFI | EC₅₀ (nM) | TOP MFI |
|---|---|---|---|---|
| 015c | 1.49 | 79989 | 1.58 | 50062 |
| 025c | 1.49 | 77587 | 1.34 | 51608 |
| 042c | 1.90 | 73225 | 4.21 | 54782 |
| 059c | 1.45 | 69840 | 12.03 | 19608 |
| 071c | 2.60 | 76483 | 3.31 | 46725 |
| 073c | 1.50 | 67794 | 2.12 | 47326 |
| hu1H9G4 | 1.93 | 74571 | 0.85 | 18671 |

TABLE 10
binding of anti-SIRPα chimeric antibodies to human SIRPβ and human SIRPβI

| | hSIRPβ ECD | | CHOK1-hSIRPβ | | hSIRPβI ECD | |
|---|---|---|---|---|---|---|
| Ab | EC₅₀ (nM) | TOP OD450 | EC₅₀ (nM) | TOP MFI | EC₅₀ (nM) | TOP OD450 |
| 015c | 0.04 | 2.97 | N/A | N/A | 0.40 | 3.10 |
| 025c | 0.04 | 3.10 | 0.38 | 10932 | 0.33 | 2.80 |
| 042c | 0.23 | 3.23 | 39.83 | 3913 | 0.39 | 2.69 |
| 059c | 0.03 | 2.75 | N/A | N/A | 0.70 | 3.35 |
| 071c | 1.53 | 2.98 | N/A | N/A | 0.30 | 2.66 |
| 073c | 2.14 | 3.07 | 22.95 | 2629 | 0.27 | 2.64 |
| hu1H9G4 | 0.03 | 3.22 | 0.30 | 7145 | 0.24 | 2.74 |

N/A stands for no available data.

TABLE 11
binding of anti-SIRPα chimeric antibodies
to human SIRPγ and cyno SIRPγ

| | 293F-human SIRPγ | | Cyno SIRPγ ECD | |
|---|---|---|---|---|
| Ab | EC₅₀ (nM) | TOP MFI | EC₅₀ (nM) | TOP OD450 |
| 005c | N/A | N/A | No specific binding | |
| 015c | 0.09 | 3300 | N/A | N/A |
| 025c | 0.11 | 3223 | 0.36 | 3.07 |
| 042c | 49.55 | 1700 | 20.53 | 1.22 |
| 059c | 34.07 | 2859 | N/A | N/A |
| 071c | 77.78 | 730.6 | N/A | N/A |
| 073c | 12.57 | 727.9 | 45.52 | 0.92 |
| hu1H9G4 | 0.52 | 2715 | 0.48 | 1.81 |

N/A stands for no available data.

TABLE 12
binding of anti-SIRPα chimeric antibodies
to cyno SIRPα and mouse SIRPα

| | |
|---|---|
| C57BL/6 mouse SIRPα | CHOK1-Cyno SIRPα |

| Ab | ECD, OD450 at 100 nM | EC₅₀ (nM) | TOP MFI |
|---|---|---|---|
| 015c | No specific binding | 0.69 | 29658 |
| 025c | | 0.69 | 30840 |
| 042c | | 2.95 | 32393 |
| 059c | | 17.21 | 17389 |
| 071c | | 2.64 | 32321 |
| 073c | | 1.76 | 30735 |
| hu1H9G4 | 0.23 | 0.72 | 14887 |

TABLE 13
CD47/SIRPα interaction blocking activity
of anti-SIRPα chimeric antibodies

| | Human CD47/SIRPα v1 | | Human CD47/SIRPα v2 | |
|---|---|---|---|---|
| | interaction blocking | | interaction blocking | |
| | IC₅₀ | TOP blocking | IC₅₀ | TOP blocking |
| Ab | (nM) | (%) | (nM) | (%) |
| 005c | No blocking | | N/A | N/A |
| 015c | 2.32 | 96.4 | 2.45 | 96.71 |
| 025c | 2.12 | 96.5 | 2.21 | 96.54 |
| 042c | 3.60 | 97.1 | 4.49 | 94.64 |
| 059c | 2.63 | 97.0 | 54.06 | 84.4 |
| 071c | 2.86 | 94.1 | 5.10 | 76.35 |
| 073c | 2.74 | 95.1 | 4.92 | 82.76 |
| hu1H9G4 | 1.91 | 97.5 | 5.74 | 98.2 |

N/A stands for no available data.

TABLE 14
SHP-1 recruitment blocking activity
of anti-SIRPα chimeric antibodies

SHP-1 recruitment blocking

| Ab | IC₅₀ (nM) | TOP blocking (%) |
|---|---|---|
| 005c | 3.85 | 76.40 |
| 015c | 0.09 | 73.51 |
| 025c | 0.28 | 84.53 |
| 042c | 0.99 | 70.00 |
| 059c | 0.07 | 66.92 |
| 071c | 2.97 | 44.50 |
| 073c | 5.66 | 51.25 |
| hu1H9G4 | 0.10 | 59.49 |

TABLE 15
anti-SIRPα chimeric antibodies affinity summary

| Antigen | Antibody | ka (1/Ms) | kd (1/s) | KD (M) |
|---|---|---|---|---|
| Human SIRPα v1 | 015c | 8.97E+05 | 3.03E−04 | 3.388−10 |
| | 025c | 6.36E+05 | 1.56E−03 | 2.46E−09 |
| | 042c | S.93E+05 | 5.45E−03 | 9.20E−09 |
| | 059c | 9.44E+05 | 2.13E−03 | 2.25E−09 |
| | 071c | 5.48E+05 | 3.27E−03 | 5.97E−09 |
| | 073c | 4.99E+05 | 3.03E−03 | 6.07E−09 |
| | hu1H9G4 | 7.95E+05 | 2.77E−03 | 3.48E−09 |
| Human SIRPα v2 | 015c | 1.24E+06 | 1.17E−03 | 9.375−10 |
| | 025c | 1.10E+06 | 2.93E−03 | 2.67E−09 |
| | 042c | 6.16E+05 | 3.78E−03 | 6.14E−09 |

TABLE 16
anti-SIRPα chimeric antibodies epitope binning summary
% Competition

| | [symbol] | | |
|---|---|---|---|
| [symbol] | 025c | 042c | hu1H9G4 |
| 025c | 93.5 | 19.1 | 15.3 |
| 042c | 8.9 | 93.9 | 29.8 |
| hu1H9G4 | 14.5 | 82.6 | 90.5 |
| 073c | 9.1 | 93.6 | 37.7 |

[symbol]

indicates data missing or illegible when filed

TABLE 17
binding of anti-SIRPα humanized antibodies to SIRP family members

| | CHOK1-hSIRPα v1 | | CHOK1-hSIRPα v2 | | CHOK1-hSIRPβ | | 293F-hSIRPγ | |
|---|---|---|---|---|---|---|---|---|
| Ab | EC₅₀ (nM) | TOP MFI | EC₅₀ (nM) | TOP MFI | EC₅₀ (nM) | TOP MFI | EC₅₀ (nM) | TOP MFI |
| hu025.021 | 1.08 | 56405 | 0.87 | 46450 | 72043 | 0.68 | 0.21 | 22134 |
| hu025.023 | 1.13 | 57807 | 0.81 | 45824 | 72753 | 0.66 | 0.20 | 22420 |
| hu025.033 | 0.99 | 53789 | 0.62 | 39793 | 69743 | 0.80 | 0.21 | 20933 |
| hu025.059 | 1.05 | 59377 | 0.75 | 47252 | 73084 | 0.67 | 0.20 | 22666 |
| hu025.060 | 0.84 | 61472 | 0.80 | 48846 | 71249 | 0.75 | 0.22 | 22192 |
| 025c | 1.22 | 64734 | 0.99 | 52966 | 72279 | 0.65 | 0.20 | 22782 |

TABLE 18
CD47/SIRPα interaction blocking activity of anti-SIRPα
humanized antibodies measured by competitive ELISA

| | Human CD47/SIRPα v1 | | Human CD47/SIRPα v2 | |
|---|---|---|---|---|
| | interaction blocking | | interaction blocking | |
| | IC₅₀ | TOP blocking | IC₅₀ | TOP blocking |
| Ab | (nM) | (%) | (nM) | (%) |
| 025c | 2.03 | 97.5 | 1.86 | 98.6 |
| hu025.021 | 2.07 | 97.7 | 1.96 | 98.3 |
| hu025.023 | 2.60 | 97.2 | 2.07 | 98.0 |
| hu025.033 | 2.22 | 97.2 | 1.88 | 98.3 |
| hu025.059 | 2.48 | 97.1 | 2.03 | 98.3 |
| hu025.060 | 2.15 | 95.2 | 1.88 | 97.0 |

TABLE 19
CD47/SIRPα interaction blocking activity of anti-SIRPα
humanized antibodies measured by competitive FACS

| | Human CD47/SIRPα v1 | | Human CD47/SIRPα v2 | |
|---|---|---|---|---|
| | interaction blocking | | interaction blocking | |
| | IC₅₀ | TOP blocking | IC₅₀ | TOP blocking |
| Ab | (nM) | (%) | (nM) | (%) |
| 025c | 0.40 | 97.6 | 0.63 | 99.7 |
| hu025.023 | 0.57 | 97.7 | 0.75 | 100 |
| hu025.060 | 0.53 | 98.7 | 0.77 | 99.8 |
| HEFLB | 0.94 | 97.6 | No blocking | |
| hu1H9G4 | 0.44 | 97.2 | 5.61 | 98.7 |

TABLE 20
SHP-1 recruitment blocking activity
of anti-SIRPα humanized antibodies

SHP-1 recruitment blocking

| Ab | IC₅₀ (nM) | TOP blocking (%) |
|---|---|---|
| hu025.021 | 0.10 | 86.28 |
| hu025.023 | 0.10 | 85.55 |
| hu025.033 | 0.19 | 84.53 |
| hu025.059 | 0.10 | 78.85 |
| hu025.060 | 0.09 | 87.52 |
| 025c | 0.08 | 86.98 |

TABLE 21
anti-SIRPα humanized antibodies affinity summary

| Antigen | Antibody | ka (1/Ms) | kd (1/s) | KD (M) |
|---|---|---|---|---|
| Human SIRPα v1 | 025c | 6.02E+05 | 9.24E−04 | 1.53E−09 |
| | hu025.21 | 5.99E+05 | 1.83E−03 | 3.05E−09 |
| | hu025.23 | 6.43E+05 | 1.72E−03 | 2.67E−09 |
| | hu025.59 | 5.36E+05 | 1.23E−03 | 2.29E−09 |
| | hu025.60 | 5.60E+05 | 1.11E−03 | 1.99E−09 |
| Human SIRPα v2 | 025c | 1.51E+06 | 2.54E−03 | 1.69E−09 |
| | hu025.21 | 1.65E+06 | 5.60E−03 | 3.39E−09 |
| | hu025.23 | 1.77E+06 | 4.92E−03 | 2.78E−09 |
| | hu025.59 | 1.42E+06 | 3.92E−03 | 2.76E−09 |
| | hu025.60 | 1.47E+06 | 3.44E−03 | 2.34E−09 |

1. An antibody or an antigen-binding fragment thereof capable of specifically binding to human SIRPα, comprising a heavy chain variable region comprising HCDR1, HCDR2 and HCDR3, and a light chain variable region comprising LCDR1, LCDR2 and LCDR3, wherein
a) the HCDR1 comprises an amino acid sequence of DYYMS (SEQ ID NO: 1),
the HCDR2 comprises an amino acid sequence of FIKNEANGYTTESSASVKG (SEQ ID NO: 2),
the HCDR3 comprises an amino acid sequence of YDYYGSNYNWYFDA (SEQ ID NO: 3),
the LCDR1 comprises an amino acid sequence of KASQNVRTAVA (SEQ ID NO: 4), and
the LCDR2 comprises an amino acid sequence of LASKRHT (SEQ ID NO: 5), and
the LCDR3 comprises an amino acid sequence of LQHWIHPLT (SEQ ID NO: 6),
b) the HCDR1 comprises an amino acid sequence of X₁YYMH (SEQ ID NO: 18),
the HCDR2 comprises an amino acid sequence of RIDPEDX₂EX₃KYAPKFQG (SEQ ID NO: 19),
the HCDR3 comprises an amino acid sequence of GX₁₈X₄X₅Y (SEQ ID NO: 20),
the LCDR1 comprises an amino acid sequence of SASSSVSSSYLY (SEQ ID NO: 10),
LCDR2 comprises an amino acid sequence of STSNLAS (SEQ ID NO: 11), and
the LCDR3 comprises an amino acid sequence of X₆QWSSYPYT (SEQ ID NO: 21),
c) the HCDR1 comprises an amino acid sequence of TYGMS (SEQ ID NO: 22),
the HCDR2 comprises an amino acid sequence of WINTYSGVX₁₉TX₇ADDFX&G (SEQ ID NO: 38),
the HCDR3 comprises an amino acid sequence of DPHX₉YGX₁₀SPAWFX₁₁Y (SEQ ID NO: 39),
the LCDR1 comprises an amino acid sequence of X₁₂ASQX₁₃ VGIX₁₄VA (SEQ ID NO: 40),
the LCDR2 comprises an amino acid sequence of SASNRX₁₅T (SEQ ID NO: 41), and
the LCDR3 comprises an amino acid sequence of QQYSX₁₆YPX₁₇T (SEQ ID NO: 42), or
d) the HCDR1 comprises an amino acid sequence of EYVLS (SEQ ID NO: 43),
the HCDR2 comprises an amino acid sequence of EIYPGTITTYYNEKFKG (SEQ ID NO: 44),
the HCDR3 comprises an amino acid sequence of FYDYDGGWFAY (SEQ ID NO: 45),
the LCDR1 comprises an amino acid sequence of SASSSVSSSDLH (SEQ ID NO: 46),
the LCDR2 comprises an amino acid sequence of GTSNLAS (SEQ ID NO: 47), and
the LCDR3 comprises an amino acid sequence of QQWSGYPWT (SEQ ID NO: 48),
wherein X₁ is A or D; X₂ is G or A; X₃ is T or S; X₄ is L or Y; X₅ is E or A; X₆ is Y or H; X₇ is Y or C; X₈ is K or Q; X₉ is Y or S; X₁₀ is N or T or S; X₁₁ is P or A or V; X₁₂ is E or K; X₁₃ is N or I; X₁₄ is S or A; X₁₅ is Y or F; X₁₆ is S or T or A; X₁₇ is F or L; X₁₈ is S or absent; X₁₉ is S or P.

2. (canceled)

3. The antibody or antigen-binding fragment thereof of claim 1, wherein
a) the HCDR1 comprises an amino acid sequence of AYYMH (SEQ ID NO: 7) or DYYMH (SEQ ID NO: 13),
b) the HCDR2 comprises an amino acid sequence selected from the group consisting of RIDPEDGESKYAPKFQG (SEQ ID NO: 8), RIDPEDGETKYAPKFQG (SEQ ID NO: 14) and RIDPEDAETKYAPKFQG (SEQ ID NO: 17),
c) the HCDR3 comprises an amino acid sequence of GSYEY (SEQ ID NO: 9) or GLAY (SEQ ID NO: 15),
d) the LCDR1 comprises an amino acid sequence of SASSSVSSSYLY (SEQ ID NO: 10),
e) the LCDR2 comprises an amino acid sequence of STSNLAS (SEQ ID NO: 11), and
f) the LCDR3 comprises an amino acid sequence of YQWSSYPYT (SEQ ID NO: 12) or HQWSSYPYT (SEQ ID NO: 16).

4. (canceled)

5. The antibody or antigen-binding fragment thereof of claim 1, wherein
a) the HCDR1 comprises an amino acid sequence of TYGMS (SEQ ID NO: 22),
b) the HCDR2 comprises an amino acid sequence selected from the group consisting of WINTYSGVSTCADDFKG (SEQ ID NO: 23), WINTYSGVPTYADDFQG (SEQ ID NO: 28) and WINTYSGVPTYADDFKG (SEQ ID NO: 33),
c) the HCDR3 comprises an amino acid sequence selected from the group consisting of DPHSYGNSPAWFPY (SEQ ID NO: 24), DPHYYGTSPAWFAY (SEQ ID NO: 29) and DPHYYGSSPAWFVY (SEQ ID NO: 34),
d) the LCDR1 comprises an amino acid sequence selected from the group consisting of KASQNVGISVA (SEQ ID NO: 25), KASQIVGIAVA (SEQ ID NO: 30) and EASQIVGIAVA (SEQ ID NO: 35),
e) the LCDR2 comprises an amino acid sequence selected from the group consisting of SASNRYT (SEQ ID NO: 26) and SASNRFT (SEQ ID NO: 31), and
f) the LCDR3 comprises an amino acid sequence selected from the group consisting of QQYSSYPLT (SEQ ID NO: 27), QQYSTYPFT (SEQ ID NO: 32) and QQYSAYPFT (SEQ ID NO: 37).

6-7. (canceled)

8. The antibody or an antigen-binding fragment thereof of claim 1, wherein
a) a HCDR1 comprising the sequence of SEQ ID NO: 1, a HCDR2 comprising the sequence of SEQ ID NO: 2, and a HCDR3 comprising the sequence of SEQ ID NO: 3, a LCDR1 comprising the sequence of SEQ ID NO: 4, a LCDR2 comprising the sequence of SEQ ID NO: 5, and a LCDR3 comprising the sequence of SEQ ID NO: 6; or
b) a HCDR1 comprising the sequence of SEQ ID NO: 7, a HCDR2 comprising the sequence of SEQ ID NO: 8, and a HCDR3 comprising the sequence of SEQ ID NO: 9, a LCDR1 comprising the sequence of SEQ ID NO: 10, a LCDR2 comprising the sequence of SEQ ID NO: 11, and a LCDR3 comprising the sequence of SEQ ID NO: 12; or
c) a HCDR1 comprising the sequence of SEQ ID NO: 13, a HCDR2 comprising the sequence of SEQ ID NO: 14 or SEQ ID NO: 17, and a HCDR3 comprising the sequence of SEQ ID NO: 15, a LCDR1 comprising the sequence of SEQ ID NO: 10, a LCDR2 comprising the sequence of SEQ ID NO: 11, and a LCDR3 comprising the sequence of SEQ ID NO: 16; or
d) a HCDR1 comprising the sequence of SEQ ID NO: 22, a HCDR2 comprising the sequence of SEQ ID NO: 23, and a HCDR3 comprising the sequence of SEQ ID NO: 24, a LCDR 1 comprising the sequence of SEQ ID NO: 25, a LCDR2 comprising the sequence of SEQ ID NO: 26, and a LCDR3 comprising the sequence of SEQ ID NO: 27; or
e) a HCDR1 comprising the sequence of SEQ ID NO: 22, a HCDR2 comprising the sequence of SEQ ID NO: 28, and a HCDR3 comprising the sequence of SEQ ID NO: 29, a LCDR1 comprising the sequence of SEQ ID NO: 30, a LCDR2 comprising the sequence of SEQ ID NO: 31, and a LCDR3 comprising the sequence of SEQ ID NO: 32; or
f) a HCDR1 comprising the sequence of SEQ ID NO: 22, a HCDR2 comprising the sequence of SEQ ID NO: 33, and a HCDR3 comprising the sequence of SEQ ID NO: 34, a LCDR1 comprising the sequence of SEQ ID NO: 35, a LCDR2 comprising the sequence of SEQ ID NO: 26, and a LCDR3 comprising the sequence of SEQ ID NO: 37; or
g) a HCDR1 comprising the sequence of SEQ ID NO: 43, a HCDR2 comprising the sequence of SEQ ID NO: 44, and a HCDR3 comprising the sequence of SEQ ID NO: 45, a LCDR1 comprising the sequence of SEQ ID NO: 46, a LCDR2 comprising the sequence of SEQ ID NO: 47, and a LCDR3 comprising the sequence of SEQ ID NO: 48.

9. The antibody or an antigen-binding fragment thereof of claim 1, further comprising heavy chain HFR1, HFR2, HFR3 and HFR4, and light chain LFR1, LFR2, LFR3 and LFR4, wherein:
a) the HFR1 comprises EVQLVQSGAEVKKPGATVKISCKX₂₀SGFNIK (SEQ ID NO: 84) or a homologous sequence of at least 80% sequence identity thereof,
b) the HFR2 comprises WVQQAPGKGLEWIG (SEQ ID NO: 74) or a homologous sequence of at least 80% sequence identity thereof,
c) the HFR3 sequence comprises RVTITADTSTX₂₁ TAYMELSSLRSEDTAVYYCDR (SEQ ID NO: 85) or a homologous sequence of at least 80% sequence identity thereof,
d) the HFR4 comprises WGQGTLVTVSS (SEQ ID NO: 76) or a homologous sequence of at least 80% sequence identity thereof,
e) the LFR1 comprises EIVLTQSPATLSLSPGERATLSC (SEQ ID NO: 77) or a homologous sequence of at least 80% sequence identity thereof,
f) the LFR2 comprises WYQQKPGQAPKLWIY (SEQ ID NO: 78) or a homologous sequence of at least 80% sequence identity thereof,
g) the LFR3 comprises GIPARFSGSGSGTDX₂₂TLTISSLEPEDFAVYYC (SEQ ID NO: 86) or a homologous sequence of at least 80% sequence identity thereof, and
h) the LFR4 comprises FGQGTKLEIK (SEQ ID NO: 80) or a homologous sequence of at least 80% sequence identity thereof,
wherein X₂₀ is A or V; X₂₁ is N or D; X₂₂ is Y or F.

10. The antibody or antigen-binding fragment thereof of claim 9, wherein:
a) the HFR1 comprises EVQLVQSGAEVKKPGATVKISCKASGFNIK (SEQ ID NO: 83) or EVQLVQSGAEVKKPGATVKISCKVSGFNIK (SEQ ID NO: 73), or a homologous sequence of at least 80% sequence identity thereof, and/or
b) the HFR2 comprises WVQQAPGKGLEWIG (SEQ ID NO: 74) or a homologous sequence of at least 80% sequence identity thereof, and/or
c) the HFR3 sequence comprises RVTITADTSTNTAYMELSSLRSEDTAVYYCDR (SEQ ID NO: 75) or RVTITADTSTDTAYMELSSLRSEDTAVYYCDR (SEQ ID NO: 82) or a homologous sequence of at least 80% sequence identity thereof, and/or
d) the HFR4 comprises WGQGTLVTVSS (SEQ ID NO: 76) or a homologous sequence of at least 80% sequence identity thereof, and/or
e) the LFR1 comprises EIVLTQSPATLSLSPGERATLSC (SEQ ID NO: 77) or a homologous sequence of at least 80% sequence identity thereof, and/or
f) the LFR2 comprises WYQQKPGQAPKLWIY (SEQ ID NO: 78) or a homologous sequence of at least 80% sequence identity thereof, and/or
g) the LFR3 comprises GIPARFSGSGSGTDYTLTISSLEPEDFAVYYC (SEQ ID NO: 79) or GIPARFSGSGSGTDFTLTISSLEPEDFAVYYC (SEQ ID NO: 81) or a homologous sequence of at least 80% sequence identity thereof, and/or
h) the LFR4 comprises FGQGTKLEIK (SEQ ID NO: 80) or a homologous sequence of at least 80% sequence identity thereof.

11. The antibody or an antigen-binding fragment thereof of claim 1, wherein the heavy chain variable region comprises the sequence selected from the group consisting of SEQ ID NO: 63, SEQ ID NO: 65 and SEQ ID NO: 67, and a homologous sequence thereof having at least 80% sequence identity yet retaining specific binding affinity to human SIRPα; and the light chain variable region comprises the sequence selected from the group consisting of SEQ ID NO: 64 and SEQ ID NO: 66, and a homologous sequence thereof having at least 80% sequence identity yet retaining specific binding affinity to human SIRPα.

12. (canceled)

13. The antibody or an antigen-binding fragment thereof of claim 1, wherein
a) the heavy chain variable region comprises the sequence of SEQ ID NO: 49 and the light chain variable region comprises the sequence of SEQ ID NO: 50; or
b) the heavy chain variable region comprises the sequence of SEQ ID NO: 51 and the light chain variable region comprises the sequence of SEQ ID NO: 52; or
c) the heavy chain variable region comprises the sequence of SEQ ID NO: 53 and the light chain variable region comprises the sequence of SEQ ID NO: 54; or
d) the heavy chain variable region comprises the sequence of SEQ ID NO: 55 and the light chain variable region comprises the sequence of SEQ ID NO: 56; or
e) the heavy chain variable region comprises the sequence of SEQ ID NO: 57 and the light chain variable region comprises the sequence of SEQ ID NO: 58; or
f) the heavy chain variable region comprises the sequence of SEQ ID NO: 59 and the light chain variable region comprises the sequence of SEQ ID NO: 60; or
g) the heavy chain variable region comprises the sequence of SEQ ID NO: 61 and the light chain variable region comprises the sequence of SEQ ID NO: 62; or
h) the heavy chain variable region comprises the sequence of SEQ ID NO: 63 and the light chain variable region comprises the sequence of SEQ ID NO: 64; or
i) the heavy chain variable region comprises the sequence of SEQ ID NO: 63 and the light chain variable region comprises the sequence of SEQ ID NO: 66; or
j) the heavy chain variable region comprises the sequence of SEQ ID NO: 65 and the light chain variable region comprises the sequence of SEQ ID NO: 64; or
k) the heavy chain variable region comprises the sequence of SEQ ID NO: 67 and the light chain variable region comprises the sequence of SEQ ID NO: 64; or
l) the heavy chain variable region comprises the sequence of SEQ ID NO: 67 and the light chain variable region comprises the sequence of SEQ ID NO: 66.

14-15. (canceled)

16. The antibody or an antigen-binding fragment thereof of claim 1, further comprising an Fc region, optionally an Fc region of human immunoglobulin (Ig), or optionally an Fc region of human IgG.

17. The antibody or an antigen-binding fragment thereof of claim 16, wherein the Fc region is derived from human IgG4, optionally wherein the Fc region derived from human IgG4 comprises a S228P mutation and/or a L235E mutation.

18. (canceled)

19. The antibody or an antigen-binding fragment thereof of claim 1, which is
a) humanized;
b) a monoclonal antibody, a bispecific antibody, a multi-specific antibody, a recombinant antibody, a chimeric antibody, a labeled antibody, a bivalent antibody, an anti-idiotypic antibody or a fusion protein; and/or
c) a diabody, a Fab, a Fab′, a F(ab′)₂, a Fd, an Fv fragment, a disulfide stabilized FV fragment (dsFv), a (dsFv)₂, a bispecific dsFv (dsFv-dsFv′), a disulfide stabilized diabody (ds diabody), a single-chain antibody molecule (scFv), and scFv dimer (bivalent diabody), a multispecific antibody, a camelized single domain antibody, a nanobody, a domain antibody, or a bivalent domain antibody.

20-22. (canceled)

23. An antibody or an antigen-binding fragment thereof which competes for binding to human SIRPα with an antibody comprising:
a) a heavy chain variable region comprising the sequence of SEQ ID NO: 53, and a light chain variable region comprising the sequence of SEQ ID NO: 54;
b) a heavy chain variable region comprising the sequence of SEQ ID NO: 55, and a light chain variable region comprising the sequence od SEQ ID NO: 56; and/or
c) a heavy chain variable region comprising the sequence of SEQ ID NO: 61 and a light chain variable region comprising the sequence of SEQ ID NO: 62.

24-25. (canceled)

26. The antibody or an antigen-binding fragment thereof of claim 1, which is bispecific, wherein
a) the antibody or an antigen-binding fragment thereof is capable of specifically binding to a second antigen other than SIRPα, optionally wherein the second antigen is a tumor antigen, tumor surface antigen, an inflammatory antigen, an antigen of an infectious microorganism; or
b) the antibody or an antigen-binding fragment thereof is capable of specifically binding to a second epitope on SIRPα.

27-29. (canceled)

30. The antibody or an antigen-binding fragment thereof of claim 1, which is linked to one or more conjugate moieties, optionally wherein the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety, or other anticancer drugs.

31. (canceled)

32. A pharmaceutical composition comprising the antibody or an antigen-binding fragment thereof of claim 1, and one or more pharmaceutically acceptable carriers.

33. An isolated polynucleotide encoding the antibody or an antigen-binding fragment thereof of claim 1.

34. A vector comprising the isolated polynucleotide of claim 33.

35. A host cell comprising the vector of claim 34.

36-39. (canceled)

40. A method of treating, preventing or alleviating a disease disorder or condition that can be benefited from induced phagocytosis of a target cell in a subject, comprising administering to the subject a therapeutically effective amount of the antibody or an antigen-binding fragment thereof of claim 1, optionally in combination with a target antibody that specifically binds to a target antigen on the target cell.

41. A method of treating, preventing or alleviating a SIRPα related disease disorder or condition in a subject, comprising administering to the subject a therapeutically effective amount of the antibody or an antigen-binding fragment thereof of claim 1, optionally in combination with a target antibody that specifically binds to a target antigen on the target cell.

42. The method of claim 41, wherein the target cell is a CD47 expressing cell.

43-45. (canceled)

46. The method of claim 41, wherein the disease, disorder or condition is cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, or arthritis, optionally wherein:
a) the cancer is anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, head and neck squamous cell carcinoma, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphocytic, non-Hodgkin lymphoma (NHL), multiple myeloma, T or B cell lymphoma, GI organ interstitialoma, soft tissue tumor, hepatocellular carcinoma, and adenocarcinoma; and/or
b) the cancer is a CD47-positive cancer.

47-48. (canceled)

49. The method of claim 41, wherein the subject is human.

50-59. (canceled)

60. A method of potentiating a target antibody in treating a disease, disorder or condition in a subject, comprising: administering to the subject a therapeutically effective amount of the antibody or an antigen-binding fragment thereof of claim 1, in combination with the target antibody, thereby potentiating the target antibody in treating the disease, disorder or condition in the subject, optionally wherein the disease, disorder or condition is immune related disease or disorder, tumors and cancers, autoimmune diseases, or infectious disease.

61-63. (canceled)
